(19) United States
(12) Patent Application Publication
(10) Pub. No.: US 2015/0344580 A1
(43) Pub. Date: Dec. 3, 2015

(54) Human Monoclonal Antibodies Against Human Chemokine Receptor CCR7

(71) Applicants: Eldar Kim, (US); MSM Protein Technologies, Woburn, MA (US)

(72) Inventors: Svetlana Abbasova, Moscow Region (RU); Viktoriia Vasilyeva, Somerville, MA (US); Andrey Ulitin, Moscow Region, Pushchino (RU); Olga Rimkevich, Boston, MA (US); Valery Solovyev, Moscow Region, Pushchino (RU); Tajib Mirzabeko, Newton, MA (US); Roman Mikhaylov, Moscow Region, Pushchino (RU); David Kreimer, Medford, MA (US); Eldar Kim, Belmont, MA (US)

(21) Appl. No.: 14/404,701

(22) PCT Filed: Mar. 13, 2013
(86) PCT No.: PCT/US13/30865

§ 371 Date: Dec. 1, 2014

Related U.S. Application Data
(60) Provisional application No. 61/655,750, filed on Jun. 5, 2012.

(51) Int. Cl. C07K 16/28

(52) U.S. Cl. CPC C07K 16/2866; C07K 2317/56; C07K 2317/565; C07K 2317/51; C07K 2317/515; C07K 2317/21; C07K 2317/92; C07K 2317/76; C07K 2317/94

(57) ABSTRACT

Aspects of this invention include fully human antibodies or fragments thereof that bind specifically to human CCR7 receptor. Such antibodies or fragments thereof can be used to treat disorders involving over function of the CCR7 receptor, including cancers. Other uses include detection of human CCR7 receptor in biological samples for diagnostic or evaluative purposes. Fully human antibodies against human CCR7 therefore can be used to diagnose disorders involving CCR7. Further, antibodies of this invention can be useful for treating disorders involving CCR7 by inhibiting binding of native chemokines to the CCR7, and thereby decrease effects of those chemokines. Anti-CCR7 antibodies of this invention can also be used as specific targeting agents to bring toxic agents to CCR7-expressing cells, to inhibit chemotaxis, and therefore can be effective therapeutic agents.

### PRIORITY CLAIM

This application claims priority to U.S. Provisional Patent Application No. 61/655,750, filed 5 Jun. 2012, entitled “Human Monoclonal Antibodies Against Human Chemokine Receptor CCR7,” Svetlana Abbasova, et al., inventors. This provisional application is incorporated herein fully by reference.

### FIELD OF THE INVENTION

This invention relates to antibodies against human G-protein coupled receptors (GPCRs). Particularly, this invention relates to fully human antibodies and fragments thereof directed against GPCRs, as well as conjugates of such antibodies and fragments thereof with toxins or radionuclides aimed at killing cells to which the conjugates bind. More particularly, this invention relates to fully human antibodies and fragments thereof directed against the human chemokine receptor CCR7, and to the conjugates of such antibodies.

### BACKGROUND

Chemokines are molecules having diverse function. They are extracellular molecules that can initiate and/or maintain numerous cell processes, including chemotaxis, cell growth and in some cases, tumor growth, homing of malignant cells and metastasis. Chemokines can act by binding to, activating, or inhibiting receptors known as chemokine receptors. Chemokine receptors are in the class of G-protein coupled receptors (GPCRs) that are multispanning membrane proteins, in which the protein has one or more regions that span a cellular membrane.

### SUMMARY

We disclose fully human antibodies that can specifically bind to human chemokine receptor CCR7 on the surfaces of living cells. We disclose 7 different antibodies with different variable domain (CDR3; CDR stands for complementarity determining region) sequences. These fully human antibodies can be used as therapeutics for the treatment of different types of cancer, inflammation, and other diseases. These fully human antibodies selectively bind to human CCR7, and include antibodies having antagonist (neutralizing) properties. These antibodies can be used in the IgG4 format (IgG stands for immunoglobulin G) that generally does not induce killing of a cell to which the antibodies bind in the organism, or other IgG format, such as the IgG1 format. The IgG1 format is an antibody subclass capable of inducing antibody-dependent cellular cytotoxicity (ADCC) and complement-dependent cytotoxicity (CDC) thus causing the death of a cell to which IgG1 is bound. These antibodies can also be conjugated with toxins or radionuclides aimed at killing cells to which the conjugates bind; the form of antibody-based drug known in the filed as antibody-drug conjugate (ADC), which stands for Antibody-Drug Conjugate. Cancers such as Chronic Lymphocytic Leukemia (CLL), T-cell Acute Lymphoblastic Leukemia (T-ALL), Follicular Lymphoma (FL), Mantle Cell Lymphoma (MCL), Head and Neck Cancer (HNC), Non-Small Cell Lung Cancer (NSCLC), Breast Cancer, Gastric Cancer, Melanoma and other types of cancer that express chemokine receptor CCR7 are therapeutic targets for the fully human anti-CCR7 antibodies and fragments thereof or ADC of this invention. Various inflammatory conditions and diseases in which CCR7 is implicated, such as Rheumatoid Arthritis (RA) can also be treated with the fully human anti-CCR7 antibodies and fragments thereof or ADC of this invention.

### BRIEF DESCRIPTION OF THE FIGURES

This invention is described with reference to specific embodiments thereof. Other features and aspects of this invention can be appreciated with reference to the Figures, in which:

FIG. 1 depicts a graph of fluorescence of cells expressing CCR7 or other GPCRs, and labeled with commercial anti-respective GPCR antibodies conjugated with fluorescent dye phycoerythrin (PE).

FIG. 1A depicts original fluorescence flow cytometry data obtained using Guava PCA-96 instrument for human CCR7 expressing CHO cells and for the CCR7 Target Presentation Material in the form of Golik of this invention

FIG. 1B depicts original fluorescence flow cytometry data obtained using Guava PCA-96 instrument for the CCR7 Target Presentation Material prepared at various Solubilization Buffer composition in the form of FMPLs of this invention.

FIG. 1C depicts a graph of fluorescence of CHO cells expressing human CCR7 at varying concentration of serum from mice immunized with 2, 5, 10, or 20 μL of CCR7-Golik of this invention according the immunization protocol of this invention, as compared to PBS (vehicle, 20 μL).

FIG. 1D depicts a graph of fluorescence of CHO cells expressing human CCR7 at varying concentration of serum from mice immunized according the immunization protocol of this invention using the CCR7 Target Presentation Material in the form of Golik of this invention.

FIG. 1E depicts a graph of fluorescence of BHK cells expressing human CCR7 at varying concentration of serum from mice immunized with the CCR7 Target Presentation Material in the form of Golik of this invention.

FIG. 1F depicts a graph of fluorescence of CHO and BHK cells expressing human CCR7 and CHO parental cells at varying concentration of serum from best-responding mouse (Group20/#2) immunized with for the CCR7 Target Presentation Material in the form of Golik of this invention.

FIG. 1G depicts a graph of fluorescence of CHO cells expressing human CCR7 vs. CHO parental cells (CHO Host) in the presence of the Serum (at 1/100 dilution) from the best mouse responder (Group20/#2) to immunization with the CCR7-Golik of this invention, as compared to fluorescence of these cells in the presence of Serum (at the same dilution) from a control mouse immunized with vehicle (Group Control 20/#1).

FIG. 2 depicts a graph of fluorescence of cells expressing CCR7 or other GPCRs, and labeled with human antibody IgG1 MSM-R707 of this invention. Columns 1-19 show results for the cells: (1) R1610-human CXCR1; (2) Cf2th-human CXCR2; (3) R1610-human CXCR3; (4) Cf2th-human CXCR4; (5) CHO-human CXCR5; (6) CHO-human CXCR6; (7) CHO-human CXCR7; (8) CHO-human CCR3; (9) CHO-human CCR4; (10) CHO-human CCR5; (11) CHO-human CCR6; (12) CHO-cyno CCR6; (13) CHO-mouse CCR6; (14) CHO-human CCR7; (15) R1610-human CCR7; (16) CHO-mouse CCR7; (17) R1610-human CCR9; (18) CHO-human CCR10; and (19) CHO-cyno CXCR3, respectively.

FIG. 3 depicts a graph of fluorescence of cells as in FIG. 2 expressing CCR7 or other GPCRs, and labeled with human antibody IgG1 MSM-R707B of this invention.

FIG. 4 depicts a graph of fluorescence of cells as in FIG. 2 expressing CCR7 or other GPCRs, and labeled with human antibody IgG1 MSM-R707BR of this invention.

FIG. 5 depicts a graph of fluorescence of cells as in FIG. 2 expressing CCR7 or other GPCRs, and labeled with human antibody IgG1 MSM-R707BL of this invention.

FIG. 6 depicts a graph of fluorescence of cells as in FIG. 2 expressing CCR7 or other GPCRs, and labeled with human antibody IgG1 MSM-R7707BI of this invention.

FIG. 7 depicts a graph of fluorescence of cells as in FIG. 2 expressing CCR7 or other GPCRs, and labeled with human antibody IgG1 MSM-R710 of this invention.

FIG. 8 depicts a graph of fluorescence of cells as in FIG. 2 expressing CCR7 or other GPCRs, and labeled with human antibody IgG1 MSM-R735 of this invention.

FIG. 9 depicts a graph of fluorescence of CHO cells expressing human CCR7 in the presence of varying concentration of IgG1 antibodies of this invention MSM-R707, R707BL, R707BI, R707BR, and R707B, and labeled with a commercial anti-human Fe PE-conjugate, as compared with CHO-parental cells for one of the antibodies, MSM-R707.

FIG. 10 depicts a graph of fluorescence of BHK cells expressing human CCR7 and BHK parental cells in the presence of varying concentration of IgG1 antibodies of this invention MSM-R710 and stained as in FIG. 9.

FIG. 11 depicts a graph of fluorescence of CHO cells expressing either human CCR7 or mouse CCR7 in the presence of varying concentration of IgG1 antibodies of this invention MSM-R707 (for CHO-human CCR7 cells data of another experiment that shown in FIG. 9 are provided) or MSM-R735 and stained as in FIG. 9.

FIG. 12 depicts a graph of inhibition of the increase in the intracellular Ca concentration in response to addition of human CCR7 ligands, CCL19 or CCL21, to Chem-1 cells expressing human CCR7 by IgG1 antibodies MSM-R707, R710, and R735 (at 1 μM concentration) of this invention.

FIG. 13 depicts a graph of inhibition of the increase in the intracellular Ca concentration in response to addition of human CCL19 to Chem-1 cells expressing human CCR7 in the presence of inhibiting IgG1 antibodies MSM-R707 at varying concentration.

FIG. 14 depicts a graph of inhibition of the increase in the intracellular Ca concentration in response to addition of human CCL21 to Chem-1 cells expressing human CCR7 in the presence of inhibiting IgG1 antibodies MSM-R707 at varying concentration.

FIG. 15 depicts the amino acid sequence alignment of MSM-R707 and its derivatives of this invention.

FIG. 16 depicts amino acid sequence alignment of CCR7 from human, Cyno [molgus monkey], marmoset monkey and mouse.

FIG. 17 depicts amino acid sequence alignment of ligands for CCR7 from human, Mulatta, Cyno, and Marmoset monkey and mouse.

FIGS. 18 A-C depict cells stained with human monoclonal antibodies against human CCR7 in CD4-positive pools and CD-4 negative pools of cells. FIG. 18A depicts staining of mouse splenocytes. FIG. 18B depicts staining of Cynomolgus PBMC. FIG. 18C depicts staining of human PBMCs.

FIGS. 19A-B depict graphs of fluorescence staining by antibodies of this invention to B-CLL cells (FIG. 19A) and B-PLL cells (FIG. 19B).

FIGS. 20A-C depict cell sorter data of human anti-CCR7 antibodies. FIG. 20A depicts binding to mouse splenocytes. FIG. 20B depicts binding to Cyno PBMC. FIG. 20 C depicts binding to human PBMCs.

FIG. 21 depicts a graph of IgG concentration (horizontal axis) versus binding to human-CCR7 expressing Chinese Hamster Ovary (CHO) cells. Open circles represent IgG R707 (EC₅₀ of about 4.2 nM), gray squares represent IgG R707B1 (EC₅₀ of about 6.7 nM), and black squares represent IgG R707B (EC₅₀ of about 8.1 nM). There was no observed staining of parental CHO cells (not expressing CCR7; not shown).

FIGS. 22A-B depict graphs of IgG concentration (horizontal axis) versus binding to CCR7-expressing CHO cells. FIG. 22A depicts binding to mouse CCR7-CHO cells. The upper curve (shaded circles) depicts binding of R707 of this invention (EC₅₀ of about 4.2 nM), Filled circles depict binding of R735 of this invention (EC₅₀ of about 2.4 nM). In contrast, human IgG isotype, and three prior art antibodies show only limited binding. FIG. 22B depicts binding to human CCR7-CHO cells. R707 and R735 have EC₅₀s of about 4.2 and 3.7 nM, respectively, whereas other IgG isotype or prior art antibodies show substantially less binding.

FIGS. 23A-B depict graphs of anti-CCR7 antibodies of this invention (horizontal axis) versus fluorescence staining of JVM-13 (FIG. 23A) and CLL-ATT (FIG. 23B) cells.

FIG. 24A-J depict graphs of data obtained using a cell sorter. To row: JVM-13 cells; bottom row, CLL-ATT cells. FIG. 24A depicts cells exposed to IgG1 FIGS. 24B, C, D, and E depict cells bound to mouse anti-human CCR7 (prior art), and R704, R707 and R735, respectively. FIG. 25F depicts cells exposed to a non-binding IgG1 antibody (negative control). FIG. 24G depicts binding of CLL-ATT cells to mouse anti-human CCR7, and FIGS. 24H, I, and J depict binding of R704, R705 and R735, respectively to CLL-ATT cells.

FIGS. 25A-B depict tables of data on binding affinities (in μg/mL and nM) of MAB 197 (prior art) and R704, R767 and R735 of this invention to different cell types. FIG. 25A depicts binding to JVM-13 and CCL-ATT cells. FIG. 25B depicts binding to BKH/CCR7 cells.

FIGS. 26A-B depicts steps of a cytotoxicity assay used to evaluate efficacy of anti-CCR7 antibodies of this invention. FIG. 26A depicts a CCR7+ cell, with CCR7 GPCR depicted traversing the cell membrane with an anti-CCR7 antibody binding thereto. As shown, one portion of the CCR7 antibody binds to the CCR7 molecule. Another portion of the anti-CCR7 antibody is shown binding to a Fab-ZAP compound (containing Saporin, a plant toxin). FIG. 26B depicts a CD22+ PSMA+ cell with the GPCR shown traversing the cell membrane. Either anti-Prostate Specific Membrane Antigen (PSMA) or anti CD22 antibodies are shown close to the GPCR, and a Fab-ZAP compound is shown binding to a portion of the antibodies.

FIG. 27 depicts a flow chart for a method for carrying out a mouse Fab-ZAP cytotoxicity assay.

FIGS. 28A-B depict graphs of the log of the antibody concentration. (horizontal axis) versus the percent cell viability (vertical axis). FIG. 28A depicts effects of anti-CCR7 antibodies of this invention, mouse IgG1 and anti-human CD22 monoclonal antibodies on cell viability. FIG. 28B depicts the effects of antibodies on viability of CLL-ATT (B-CLL) cells.

FIG. 29 depicts a flow chart for a method for carrying out a human Fab-ZAP cytotoxicity assay.

FIG. 30 depicts a graph of effects of R704, R707, R735 of this invention and a non-binding human IgG1 (negative control) and mouse MAB 197 on JVM-13 (B-PLL) cells.

FIGS. 31A-B depict graphs of the log IgG concentration (horizontal axis) versus % cell viability (vertical axis) in C4-2 prostate cells and JVM-13 cells. For the C4-2 cells (FIG. 31A), anti PSMA monoclonal antibodies decreased cell viability, whereas human IgG1 (negative control) did not. In the JVM-13 cells (FIG. 31B), human anti CCR7 antibody (R735) decreased cell viability, whereas the control human IgG1 did not.

FIG. 32 depicts a flow chart for a method of carrying out a receptor internalization assay useful for determining effects of anti-CCR7 antibodies of this invention.

FIG. 33 depicts graphs of incubation time (horizontal axis) versus percent of maximal binding (reflecting internalization of the receptor) to CLL-AAT (B-CLL) cells. IgG isotype mouse control antibody did not produce internalization, whereas R707, and R735 did.

FIG. 34 depicts graphs of incubation time (horizontal axis) versus percent binding (reflecting internalization of the receptor) to C4-2 prostate cells. Mouse or human IgG isotypes showed no internalization, whereas mouse anti-PSMA mAb 3.9 and human anti-PSMA mAb 006 did.

FIG. 35 depicts a graph of IgG concentration (in nM; horizontal axis) versus inhibition of Calcium flux induced by CCL19 in reporter cells by prior art IgG antibody. The IC₅₀ is about 10 nM).

FIGS. 36A-B depict IgG concentration (in nM; horizontal axis) versus inhibition of calcium flux induced by CCL19 in reporter cells by R707 (FIG. 36A) and R735 (FIG. 36B) of this invention. The IC₅₀s for these anti-CCR7 antibodies was 20 nM and 67 nM, respectively. These results show that antibodies of this invention are effective in inhibiting the normal cellular signaling.

FIG. 37A-B depict graphs of IgG concentration (in nM; horizontal axis) as a function of time of heat treatment of anti-CCR7 antibodies of this invention. FIG. 37A shows results for R707, demonstrating little or no loss of binding ability due to heat treatment. FIG. 37B depicts little or no effect of heat treatment on binding of R735 of this invention.

FIGS. 38A-D depict photographs of SDS-polyacrylamide gels showing effects of heat treatment on R707 (lanes 1-2, R735 (lanes 3-4) and control (MDX-1338; lanes 5) and Rituximab (lanes 6). These results show that without heat treatment (FIGS. 38A and 38C), the mobilities of all of the antibodies were very similar. FIGS. 38B and 38D show that heat treatment (12 hrs at 40° C.) did not alter the mobilities of any of the antibodies studied, as demonstrated by SDS-PAGE analysis either under non-reducing or reducing conditions.

FIG. 39 shows the lack of effect of trypsin treatment on various antibodies, including R707 and R735 of this invention.

FIGS. 40A-B depict graphs of IgG concentration (in nM; horizontal axis) versus binding of antibodies of this invention over storage time. FIG. 40A depicts results for R707 of this invention. FIG. 40B depicts results for R735 of this invention.

FIG. 41 depicts effects of antibodies of this invention on chemotaxis of CLL-AAT cells in response to CCL19.

FIG. 42 depicts a graph demonstrating the inhibitory effects of anti-CCR7 antibodies of this invention on inhibition of calcium flux induced by chemokines CCL19 and CCL21.

FIG. 43 depicts a graph of the effect of anti-CCR7 antibodies of this invention on CCL21-induced calcium flux.

FIG. 44 depicts a graph of binding specificity of fully human anti-human CCR7 MSM R707 monoclonal antibodies in IgG4 format.

FIG. 45 depicts a graph of binding specificity of fully human anti-human CCR7 MSM R737 monoclonal antibodies in IgG4 format.

FIG. 46 depicts a graph of inhibition by IgG4 formatted MSM R707 of calcium flux induced by CCL19 in cells expressing human CCR7.

FIG. 47 depicts a graph of inhibition by IgG4 formatted MSM R737 of calcium flux induced by CCL19 in cells expressing human CCR7.

FIGS. 48A and 48B depict photographs of polyacrylamide gels of antibodies of this invention in IgG4 format. FIG. 48A depicts a gel run under reducing conditions. B depicts a gel run under non-reducing conditions.

FIG. 49 depicts graphs of binding of IgG4 formatted anti-CCR7 antibodies of this invention (MSM R707) to cells that over-express CCR7, and to parental cells (that do not over-express CCR7).

FIG. 50 depicts graphs of binding of IgG4 formatted anti-CCR7 antibodies of this invention (MSM R735) to cells that over-express CCR7, and to parental cells (that do not over-express CCR7).

### DETAILED DESCRIPTION

### Definitions

The term “comprising” means “including but not limited to.”

The phrase “consisting of” means “includes and is limited to.”

The phrase “consisting essentially of” means “includes and is limited to elements specified, with minor additions possible.

The term “scFv” means an antibody fragment consisting of a heavy chain and a light chain linked together by a linker.

The term “Fab”, “FAB”, or “Fab”, means an antibody fragment consisting of a heavy chain and a light chain.

The term “GPCR” means “G-Protein Coupled Receptor.”

The term “CCR7” means a GPCR for which the naturally occurring ligands chemokine 19 (CCL19) and chemokine 21 (CCL21) bind.

Aspects of this invention include fully human antibodies and fragments thereof directed against CCR7 and conjugates of these antibodies or antibody fragments with toxins or radionuclides Antibody-Drug Conjugates (ADCs) aimed at destroying cells to which such ADCs binds. CCR7 is involved in cancer, and antibodies and fragments there that bind to CCR7 can result in decreased cancer growth and in suppression of homing of malignant cells and of metastases. In aspects of this invention, antibodies and fragments thereof are fully human. This provides therapeutic potential in treating human disease, because use of non-human antibodies or even humanized antibodies can produce unwanted side effects due to graft versus host immune responses to the antibodies. Thus, fully human antibodies can provide greater therapeutic index compared to other antibody-based approaches.

CCR7 is a G-Protein Coupled Receptor (GPCR) that binds to CC chemokine ligands MIP-3beta (ELC/CCL19) and 6Ckine (CCL21) (Yoshida et al. Molecular cloning of a novel human CC chemokine EBI1-ligand chemokine that is a specific functional ligand for EBI1, CCR7. J Biol Chem. 272: 13803-13809 (1997)). These ligands are expressed in the secondary lymphoid organs, and binding to CCR7 expressed in naïve T cells, B cells and dendritic cells directs migration of these cells to sites of antigen presentation (Foster et al. CCR7 coordinates the primary immune response by establishing functional microenvironments in secondary lymphoid organs. Cell, 99:23-33 (1999)). Inhibition of CCR7/ligand interactions inhibits contact sensitivity, delayed type hypersensitivity, and graft vs. host disease in experimental models (Foster et al. Id., Sasaki et al. Antagonist of secondary lymphoid-tissue chemokine (CCR ligand 21) prevents the development of chronic graft-versus-host disease in mice. J Immunol. 170: 588-596 (2003)). In addition, CCR7 expression by breast cancer, melanoma and other malignant cells are associated with lymph node metastasis (Muller et al., Involvement of chemokine receptors in breast cancer metastasis. Nature. 6824:50-56 (2001); Payne, A. S. and L. A. Cornelius. The role of chemokines in melanoma tumor growth and metastasis. J. Invest. Dermatol. 118: 915-922 (2002)).

More recently, the CCR7-CCL19 axis was shown to be implicated in homing and metastasis of malignant T-ALL cells into central nervous system, and with formation of metastasis in the brain, as described in Buonamici, S., Trimarchi, T., Ruocco, M. G., Reavie, L., Cathelin, S., Mar, B. G., Klinakis, A., Lukyanov, Y., Tseng, J. C., Sen, F., Gehrie, E., Li, M., Newcomb, E., Zavadil, J., Meruelo, D., Lipp, M., Ibrahim, S., Efstratiadis, A., Zagzag, D., Bromberg, J. S., Dustin, M. L., and Aifantis, I. (2009) Nature, 459, 1000-1004, and in Shannon, L. A., McBurney, T. M., Wells, M. A., Roth, M. E., Calloway, P. A., Bill, C. A., Islam, S., and Vines, C. M. (2012) J Biol Chem incorporated herein fully by reference.

Although the mechanisms of such action of the fully human antibodies of this invention are not completely known, there are several possible mechanisms. Ligands of CCR7, chemokine peptides CCL19 and CCL21, are expressed in lymph nodes. A signal induced by interaction of such ligands with CCR7-expressing tumor cells can result in metastatic homing of tumor cells in the lymph nodes. Blockade of CCR7 with antagonistic antibodies can prevent or inhibit the signaling, and thus can prevent or inhibit homing of tumor cells in the lymph nodes. However, this invention is not intended to be limited to a particular mechanism of action.

Even in the absence of antagonistic effects, important therapeutic effects of antibodies can arise from binding of antibodies to CCR7. In such cases, activation of ADCC and CDC mechanisms can result to the elimination of CCR7 expressing cells. Part of the mechanism is similar to the mechanism of how Rituxan™ is thought to eliminate CD20-expressing cells.

Another mode of possible therapeutic efficacy of the antibodies of this invention includes the inhibition of chemotaxis of CCR7-expressing cells due to binding of these antibodies to CCR7 and thereby inhibiting chemoattractant signals induced by the chemokine ligands CCL19 and CCL21.

CCR7 is an important receptor with a role in trafficking of B and T lymphocytes and dendritic cells to and across high endothelial venules and positioning those cells correctly in T cell zones of secondary lymphoid organs. The natural ligands of CCR7 are chemokines CCL19 (also called MIP-3beta, ELC, or Exodus-3) and CCL21 (also called 6Ckine, SLC, and Exodus-2), both biding to T-cells and actT and mDC cell types.

Binding of chemokines to their corresponding GPCRs induce cell signaling. In case of CCR7, the ligand binding induced signaling can be involved in the progression of cancer and some inflammatory diseases. Therefore the blocking of this signaling can be therapeutically useful. We have found that some human antibodies binding to CCR7 neutralize the binding of chemokines to this receptor, e.g. the signaling. We call these antibodies antagonists or neutralizing antibodies.

It should be appreciated that the above mechanisms are for purposes of illustration only, and are not considered to be the only mechanisms possible. The proper scope of this invention includes all possible mechanisms of action of the fully human antibodies of this invention.

### EMBODIMENTS OF THE INVENTION

The specific embodiments herein below are for purposes of example only, and are not intended to limit the scope of this invention. It will be appreciated that the description herein can be embodied in various ways, including but not limited to the following.

Some embodiments include a fully human monoclonal antibody in IgG1, IgG2, IgG3 or IgG4 or other immune globulin format against human chemokine receptor CCR7.

Additional embodiments include fully human antibodies against human CCR7 of any preceding embodiment, further comprising that specifically binds to human CCR7.

Additional embodiments include fully human antibodies against human CCR7 of any preceding embodiment that are essentially free of contaminants.

Further embodiments include an antibody fragment of any preceding embodiment, said fragment capable of specifically binding to human CCR7.

Additional embodiments of any preceding embodiment includes pharmaceutical compositions

comprising a fully human antibody against human CCR7 and a pharmaceutically acceptable carrier or excipient.

Further embodiments of any preceding embodiment include a library of fully human anti-human CCR7 antibodies.

Additional embodiments of any preceding embodiment include a fully human anti-CCR7 antibody having a CDR3 HC sequence selected from any of Tables 5 through 11.

Still further embodiments of any preceding embodiment include an anti-CCR7 antibody, having a heavy chain fragment having a sequence selected from the group consisting of any of Tables 5 through 11.

Additional embodiments of any preceding embodiment include a fully human anti-CCR7 antibody having a CDR3 LC sequence selected from any of Tables 5 through 11.

Still further embodiments of any preceding embodiment include an anti-CCR7 antibody, having a light chain fragment having a sequence selected from the group consisting of any of Tables 5 through 11.

Alternative embodiments of any preceding embodiment include fully human antibodies against human CCR7 being in IgG1 format.

Further embodiments of any preceding embodiment include fully human antibodies against human CCR7 selected from the group of MSM R707, MSM R707B, MSM R707BR, MSM R707BL, MSM R707 BI, MSM R710, and MSM R735

In alternative embodiments of any preceding embodiment, a fully human antibody against human CCR7 has a VH sequence encoded by SEQ ID NO.3, and the VL region is encoded by SEQ ID NO.4.

In yet further embodiments of any preceding embodiment of a fully human antibody against human CCR7, the HC amino acid sequence is SEQ ID NO.5, and the LC sequence is SEQ ID NO.6.

In still further embodiments of any preceding embodiment of a fully human antibody against human CCR7, the CDR1 HC has the amino acid sequence of SEQ ID NO.7, the CDR2 HC has the amino acid sequence of SEQ ID NO.8, the CDR3 HC has the amino acid sequence of SEQ NO.9, the CDR1 LC has the amino acid sequence of SEQ ID NO.10, the CDR2 LC has the amino acid sequence of SEQ ID NO.11, and the CDR3 LC has the amino acid sequence of SEQ ID NO.12.

In alternative embodiments of any preceding embodiment of a fully human antibody against human CCR7, the VH sequence is encoded by SEQ ID NO.13, and the VL region is encoded by SEQ ID NO.14.

Additional embodiments of any preceding embodiment of a fully human antibody against human CCR7, include the HC amino acid sequence of SEQ ID NO.15, and the LC amino acid sequence of SEQ ID NO.16.

In yet further embodiments of any preceding embodiment of a fully human antibody against human CCR7, the CDR1 HC has the amino acid sequence of SEQ ID NO.17, the CDR2 HC has the amino acid sequence of SEQ ID NO.18, the CDR3 HC has the amino acid sequence of SEQ NO.19, the CDR1 LC has the amino acid sequence of SEQ ID NO.20, the CDR2 LC has the amino acid sequence of SEQ ID NO.21, and the CDR3 LC has the amino acid sequence of SEQ ID NO.22.

In still further embodiments of any preceding embodiment of a fully human antibody against human CCR7, the VH sequence is encoded by SEQ ID NO.23, and the VL region is encoded by SEQ ID NO.24.

In additional embodiments of any preceding embodiment of a fully human antibody against human CCR7, the HC amino acid sequence is SEQ ID NO.25, and the LC amino acid sequence is SEQ ID NO.26.

In additional embodiments of any preceding embodiment of a fully human antibody against human CCR7, the CDR1 HC has the amino acid sequence of SEQ ID NO.27, the CDR2 HC has the amino acid sequence of SEQ ID NO.28, the CDR3 HC has the amino acid sequence of SEQ NO.29, the CDR1 LC has the amino acid sequence of SEQ ID NO.30, the CDR2 LC has the amino acid sequence of

SEQ ID NO.31, and the CDR3 LC has the amino acid sequence of SEQ ID NO.32. Further embodiments of any preceding embodiment include a fully human antibody against human CCR7, where the VH sequence is encoded by SEQ ID NO.33, and the VL region is encoded by SEQ ID NO.34.

In additional embodiments of any preceding embodiment of a fully human antibody against human CCR7, the HC amino acid sequence is SEQ ID NO.35, and the LC amino acid sequence is SEQ ID NO.36.

Moreover, in other embodiments of any preceding embodiment of a fully human antibody against human CCR7, the CDR1 HC has the amino acid sequence of SEQ ID NO.37, the CDR2 HC has the amino acid sequence of SEQ ID NO.38, the CDR3 HC has the amino acid sequence of SEQ NO.39, the CDR1 LC has the amino acid sequence of SEQ ID NO.40, the CDR2 LC has the amino acid sequence of SEQ ID NO.41, and the CDR3 LC has the amino acid sequence of SEQ ID NO.42.

In still other embodiments of any preceding embodiment of a fully human antibody against human CCR7, the VH sequence is encoded by SEQ ID NO.43, and the VL region is encoded by SEQ ID NO.44.

Still additional embodiments of any preceding embodiment of a fully human antibody against human CCR7 the HC amino acid sequence is SEQ ID NO.45, and the LC amino acid sequence is SEQ ID NO.46.

In alternative embodiments of any preceding embodiment of a fully human antibody against human CCR7, the CDR1 HC has the amino acid sequence of SEQ ID NO.47, the CDR2 HC has the amino acid sequence of SEQ ID NO.48, the CDR3 HC has the amino acid sequence of SEQ NO.49, the CDR1 LC has the amino acid sequence of SEQ ID NO.50, the CDR2 LC has the amino acid sequence of SEQ ID NO.51, and the CDR3 LC has the amino acid sequence of SEQ ID NO.52.

Moreover, in other embodiments of any preceding embodiment of a fully human antibody against human CCR7, the VH sequence is encoded by SEQ ID NO.53, and the VL region is encoded by SEQ ID NO.54.

In other embodiments of any preceding embodiment of a fully human antibody against human CCR7, the HC amino acid sequence is SEQ ID NO.55, and the LC amino acid sequence is SEQ ID NO.56.

Additionally, in embodiments of any preceding embodiment of a fully human antibody against human CCR7, the CDR1 HC has the amino acid sequence of SEQ ID NO.57, the CDR2 HC has the amino acid sequence of SEQ ID NO.58, the CDR3 HC has the amino acid sequence of SEQ NO.59, the CDR1 LC has the amino acid sequence of SEQ ID NO.60, the CDR2 LC has the amino acid sequence of SEQ ID NO.61, and the CDR3 LC has the amino acid sequence of SEQ ID NO.62.

In alternative embodiments of any preceding embodiment of a fully human antibody against human CCR7, the VH sequence is encoded by SEQ ID NO.63, and the VL region is encoded by SEQ ID NO.64.

In other embodiments of any preceding embodiment of a fully human antibody against human CCR7, the HC amino acid sequence is SEQ ID NO.65, and the LC amino acid sequence is SEQ ID NO.66.

Moreover, in further embodiments of any preceding embodiment of a fully human antibody against human CCR7, the CDR1 HC has the amino acid sequence of SEQ ID NO.67, the CDR2 HC has the amino acid sequence of SEQ ID NO.68, the CDR3 HC has the amino acid sequence of SEQ NO.69, the CDR1 LC has the amino acid sequence of SEQ ID NO.70, the CDR2 LC has the amino acid sequence of SEQ ID NO.71, and the CDR3 LC has the amino acid sequence of SEQ ID NO.72.

Additional embodiments of any preceding embodiment include a Fab fragment of an antibody of a fully human antibody against human CCR7, said Fab fragment capable of binding to human CCR7 with an affinity of about 1 nM to about 100 nM.

Further embodiments of any preceding embodiment include an scFv fragment of an antibody of a fully human antibody against human CCR7, said scFv fragment capable of binding to human CCR7 with an affinity of about 1 nM to about 100 nM.

Alternative embodiments of any preceding embodiment include a fully human antibody against human CCR7 having the amino acid sequence of SEQ ID NO.76 or the amino acid sequence of SEQ ID NO. 77.

In still other embodiments of any preceding embodiment, the heavy chain CDR3 region is selected from the group consisting of SEQ ID NO.78 through SEQ ID NO.148.

Further embodiments of any preceding embodiment additionally comprise a light chain CDR3 region selected from the group consisting of SEQ ID NO.149 through SEQ ID NO.154

Additional embodiments of any preceding embodiment of a fully human antibody against human CCR7, include compositions comprising an antibody or antibody fragment of any fully human antibody against human CCR7, further comprising a physiologically compatible solution.

Alternative embodiments of any preceding embodiment of a fully human antibody against human CCR7 further comprise one or more physiologically compatible excipients or binders.

In alternative embodiments of any preceding embodiment of this invention, methods for inhibiting an abnormal effect of human CCR7, include administering to a mammal in need thereof a fully human antibody against human CCR7, an antibody fragment of any fully human antibody against human CCR7, or a composition containing a fully human antibody against human CCR7 or a fragment thereof that binds to human CCR7.

Additional embodiments of any preceding embodiment include uses of a fully human antibody against human CCR7, or an antibody fragment of a fully human antibody against human CCR7, or a composition of including a fully human antibody against human CCR7, or a fragment thereof that binds to human CCR7 in the manufacture of a medicament to inhibit an abnormal effect of human CCR7.

Additional uses of any preceding embodiment of a fully human antibody against human CCR7 or a fragment thereof that binds to human CCR7, where the abnormal effect of CCR7 is abnormal cell growth in cancer.

Further embodiments of any preceding embodiment of a fully human antibody against human CCR7 include use where a cancer is selected from the group consisting of melanoma, chronic leukocytic leukemia, diffuse large B-Cell lymphoma, head and neck cancer, non-small cell lung cancer, gastric cancer, pancreatic cancer, and breast cancer.

Other embodiments of any preceding embodiment of a fully human antibody against human CCR7 or fragment thereof that binds to human CCR7 include uses where an abnormal effect is a fibrotic disease, inflammation, or multiple sclerosis.

Additional embodiments of any preceding embodiment of a fully human antibody against human CCR7 or fragment thereof that binds to human CCR7 includes uses where inflammation is of the eye.

Further embodiments of any preceding embodiment of a fully human antibody against human CCR7 or a fragment thereof that binds to human CCR7 include uses where the action of said anti-CCR7 antibody is to

inhibit the PIK3 AKT pathway; or

unblock the pro-apoptotic GSK3β FOXO1/3 pathway; or

activate the NFκB pro-survival pathway, or

inhibit the ERK 1/2 JNK pathway that enables chemotaxis; or

decrease the Rho PYK2 Coffin pathway to decrease the speed of chemotaxis; or

down regulate the NF-kB signaling pathway and should therefore down regulate Foxp3 expression.

In further alternative embodiments of any preceding embodiment of a fully human antibody against human CCR7 or a fragment thereof that binds to human CCR7, include methods of manufacturing a fully human antibody against human CCR7, comprising the steps:

producing a codon-optimized DNA plasmid encoding human CCR7;

expressing said plasmid an a cell capable of producing CCR7;

extracting said CCR7 from said cell using a detergent-containing solution;

attaching said CCR7 to a bead;

producing a library of human IgGs, Fabs, or scFvs; and

selecting from said library, antibodies that bind to human CCR7.

Other embodiments of any preceding embodiment of a fully human antibody against human CCR7 or a fragment thereof that binds to human CCR7 include kits for detecting human CCR7, comprising:

a fully human antibody directed against human CCR7;

a vial for preparing said antibody for use;

solutions for use in an in vitro assay; and

instructions for use.

There have been some attempts at immunotherapy for cancer using antibodies against chemokine receptors.

### Antibodies Against CCR7

There are some available antibodies directed against human CCR7. They include:

1. Mouse monoclonal IgG2A Clone #150503; R&D # MAB197;
2. MAB #150503 reacts with the Human CCR7. It is not cross-reactive with Mouse CCR7. It can neutralize human CCL19 in a chemotaxis assay with an IC₅₀ of about 15 nM. The antibody has in vivo efficacy in a human IPF fibroblast xenograft model (C. Hogaboam 2007, identified in the paper as R&D antibody). Injections were done every 2^{nd} day for 28 days. Dose is unknown.

Other antibodies (anti-mouse) include:

### 3. Rat IgG2A Antibody Monoclonal Clone 4B12; R&D # MAB3477;

4. MAB #4B12 reacts with the Mouse CCR7. It is not cross-reactive with Human CCR7. It can neutralize mouse CCL19 in a chemotaxis assay with an IC₅₀ of about 40 nM. The antibody has in vivo efficacy in a CCR7+ melanoma model (M. Swartz 2010, identified in the paper CCR7 neutralizing antibody). MAB dosing is unknown.
5. Anti-CCR7 MAB enhances HSC and MPC proliferation in vivo and protects mice from invasive aspergillosis (C. Hogaboam, 2010). MAB dosing-25 μg of either MAB, PI every other day for 14 days.

Unfortunately, none of the prior art antibodies against CCR7 are fully human, and therefore pose risk of causing allergic reactions. Therefore, this invention improves on the art by providing fully human antibodies directed against human CCR7. Examples of these antibodies are described herein below.

### Methods for Producing CCR7 Receptors

In certain embodiments of this invention, CCR7 receptors can be isolated from membranes of cells expressing the protein and used as an immunogen to produce CCR7-specific antibodies. In general, we used methods described in PCT International Patent Application No: PCT/US2007/003169, filed 5 Feb. 2007 (WO 2007/092457). This application is expressly incorporated herein fully by reference. The production of CCR7 in cell lines was confirmed as described under Example 1.

### Applications of Human Antibodies Against Human CCR7

Fully human anti-CCR7 antibodies and/or fragments thereof can be used as therapeutic agents for different types of cancer where CCR7 plays a role. The types of cancer include: (1) Chronic Leukocytic Leukemia and other blood cancers—T-cell Acute Lymphoblastic Leukemia (T-ALL), Follicular Lymphoma (FL), Mantle Cell Lymphoma (MCL); (2) Head and Neck cancer; (3) Non-Small Cell Lung Cancer; (4) Breast Cancer; (5) Gastric Cancer as well as other types of human cancers. In addition to cancer, anti-CCR7 antibodies and fragments thereof may be used as a therapeutics for treatment of inflammatory diseases such as (1) Rheumatoid Arthritis; (2) Inflammatory Bowel Disease; (3) Psoriasis; (4). Lupus; (5) Multiple Sclerosis, and (6) Asthma. However, it can be appreciated that other disorders involving CCR7 can be treated as well. Thus, the full scope of therapeutics involving antibodies and fragments thereof of this invention includes any disorder in which CCR7's actions are at least partially responsible for the disorder. Further descriptions of applications are included herein below. As can be appreciated from Examples 11-13 herein, anti CCR7 antibodies can inhibit binding of natural ligands of CCR7, thereby demonstrating that therapeutic uses of fully human CCR7 antibodies can be a viable alternative to existing treatments for such disorders.

### Description of Antibodies

We have identified three functional classes of human anti-human CCR7 (“anti-hCCR7”) antibodies:

1. Monoclonal Antibodies (MAbs) and fragments thereof which bind to CCR7 but do not affect its natural ligand binding properties and signaling;

2. MAbs and fragments thereof which bind to CCR7 and activate the signaling by natural ligands (agonists); and

3. Antibodies what bind to CCR7 and inhibit the binding of natural ligands CCL19 and CCL21 to CCR7 (antagonists). Therefore they are called neutralizing MABs.

Antibodies of this invention can be IgG1, IgG2, IgG3, or IgG4, or other immune globulin format. For some applications, it can be desirable to use anti-CCR7 antibodies of the IgG1 class. IgG1 and IgG3 typically have the highest affinity, IgG4 antibodies have intermediate affinity and IgG2 antibodies may have low affinity. In contrast, IgG3 antibodies are strong activators of complement, IgG1 are also high, IgG2 antibodies are less able to activate complement, and IgG4 antibodies may activate complement only weakly.

In addition to full-length antibodies, portions of antibodies of this invention can also be used to target and/or bind to CCR7. For example, as shown in the Examples below, smaller fragments, including Fab fragments, scFv, or other antibody-like structures can provide highly specific binding, with affinities in the range of about 1 nM to about 100 nM, to target molecules that may be, to a significant extent, determined by the sequence of CDR3 Heavy Chain (HC) regions.

All the antibodies were identified from phage display human antibody libraries presenting broad repertoire (up to 10¹¹) of different human antibody variable domains V1 and Vh as a fusion protein (scFvs libraries). Of the amino acid regions of antibodies of this invention, the CDR3 HC regions can be particularly useful in providing binding to CCR7. However, other amino acid regions are also useful, and include both heavy chains and light chains.

Fully human antibodies against human CCR7 of this invention that cross react with mouse CCR7 can be useful in further development of drugs affecting CCR7 in human beings for treatment of a variety of diseases and conditions. Numerous mouse models can be employed to demonstrate an efficacy of anti-CCR7 antibodies. Cross reactivity of human antibodies of this invention with mouse CCR7 can make the use of these well-established models straightforward. Therefore, data obtained in mouse models using fully human antibodies human are reasonably predictive of effects observed in human beings. Thus, the antibodies of this invention can be useful for treatment of human diseases and conditions, such as asthma, arteriosclerosis, various types and stages of cancer, including metastasis, various inflammatory conditions and others in which CCR7 and its natural ligands CCL19 and CCL21 are involved.

### Applications of Fully Human Anti-CCR7 Antibodies

Fully human antibodies and fragments thereof against CCR7 can be useful diagnostic and/or therapeutic agents in treatment of a variety of conditions in which CCR7 is overexpressed, or in which ligands for CCR7 are over-expressed or released in pathological situations. Examples of such disorders include inflammation, cancer, and fibrotic diseases. Anti-CCR7 antibodies may exert numerous effects via through blocking effects of chemokines on the CCR7, thereby inhibiting the effects of the chemokine on the cells that express CCR7. As described herein, there are several possible intracellular mechanisms of action of CCR7, whose abnormal effects can be mitigated using antibodies or fragments thereof of this invention. Such effects may be in cancer cells, fibrotic cells, regulatory T Cells (Tregs) or other cell types. Regardless of the particular mechanisms of action in any particular cell type, all such mechanisms or others are considered to be part of this invention.

### Detection of Natively Configured CCR7

In certain embodiments of this invention, anti-CCR7 antibodies can be useful for detection of expressed CCR7 in native configuration. Prior methods of determining expression CCR7 inadequately identify non-natively configured CCR7, and as such, may misrepresent the true amount of such CCR7 in a particular state. For example, RNA arrays and PCR assays (including quantitative PCR or “qPCR”) measure only the mRNA for CCR7 and do not reflect expression of the mature protein. Because CCR7 and other GPCRs are multispanning membrane proteins, misfolding of nascent protein chains may be important aspects of loss of CCR7 function and may lead to pathological conditions.

Additionally, anti-CCR7 antibodies raised against non-natively configured CCR7 may not detect mis-folded or mis-inserted CCR7 into cell membranes. Thus, using the antibodies of this invention, better understanding of the CCR7 status of patients can be achieved. In certain aspects, use of antibodies of this invention along with more routine analyses (qPCR, RNA arrays, prior art antibodies) can shed light upon the functional state of a cell's CCR7 status.

### Therapeutic Applications

In certain aspects of this invention, fully human CCR7 antibodies can be useful in treating conditions involving defects in CCR7, include cancers. In several types of cancer CCR7 can play important roles in dysregulation of cell growth and tumor metastasis. Thus, according to certain embodiments, use of fully human anti-CCR7 antibodies of this invention can bind to the CCR7 receptor. In some of these embodiments, binding of an antibody to a receptor can lead to loss of cells expressing CCR7. Whether this is by cell death or other mechanism is not crucial to the use of antibodies of this invention. In other embodiments, an anti-CCR7 antibody can act as an antagonist of the function of the CCR7 receptor, and these embodiments are useful to treat disorders in which CCR7 function is too high for normal functioning of the cell. In still further embodiments, anti-CCR7 antibodies of this invention can act as agonists and thereby increase the functioning of CCR7-dependent processes. Thus, antibodies and fragments thereof of this invention can find therapeutic use in a variety of pathological conditions, including cancer.

In certain aspects of this invention, an anti-CCR7 antibody of this invention can be selected based upon diagnostic findings. For example, in many types of cancer, CCR7 is over-expressed. It can be useful in some cases to determine whether a particular patient's cancer involves CCR7 over-expression. To determine whether CCR7 is over-expressed, a sample of the patient's tumor can be obtained through biopsy or resection of mass tumors, or by sampling blood in cases of leukemias, and CCR7 expression measured using measurement of mRNA expression or the natively configured CCR7 protein itself. Methods for measuring mRNA expression include solid phase arrays for mRNA, quantitative PCR (qPCR) or other methods known in the art. Methods for determining expression of CCR7 protein include enzyme-linked immunosorbent assays (ELISA), Western blotting or other methods known in the art. These methods need not be further described herein. Rather, persons of ordinary skill in the art can easily refer to published articles, textbooks, or laboratory manuals for details of these methods. However, with the use of the fully human antibodies against natively configured CCR7, diagnosis can be improved. As noted, using a combination of RNA expression and production of natively configured CCR7 can lead to an understanding of whether the particular defect is more related to RNA expression or rather, to misfolding, improper post-expression processing of the CCR7 or whether the CCR7 is improperly inserted into the cell membrane.

In cases in which CCR7 expression is undesirably high, the therapeutic goal can include reducing function of the CCR7 pathways. Antagonist antibodies of this invention can be particularly useful for these situations. In other situations, using antibodies that specifically bind to CCR7 can be used to reduce the numbers of CCR7 expressing cells.

Pharmaceutical compositions containing fully human anti-CCR7 antibodies are also included within the scope of this invention. Thus, a suitable composition can include one or more anti-CCR7 antibodies, a physiologically compatible solution, and one or more pharmacological excipients.

### CCR7 Rationale: Blocking Cancer Subversion of Regulatory T Cells (Tregs)

Regulatory T Cells (Tregs) exercise negative control over the immune system. Using multiple immune suppression mechanisms, Tregs can inhibit or completely shut down anti-cancer immune responses locally at the tumor site and systemically. Normal CCL19/CCL21 (CCL19/21) signaling to CCR7⁺ Tregs determines Treg chemotaxis and sets the immune system balance between immune activation and immune tolerance systemically and locally. Aberrant secretion of CCL19 or CCL21 in cancer recruits and maintains CCR7⁺ Tregs in tumor microenvironments, skewing the immune response towards cancer tolerance in the tumor microenvironment. Once in the tumor microenvironment, cancer cell secretion of TGF-β, IDO, B7-H1, IL-4, IL-10 and other factors can subvert Tregs into suppressing anti-cancer immune activity and into depleting anti-cancer immune cells. CCL19/21 mediated subversion of Tregs into immuno-suppression activates a broad, multi-factorial and potent pathway to complete cancer immune escape seen in dozens of solid tumors.

### CCR7: Rationale for Broader Efficacy of Anti-Treg Cancer Immunotherapy

CCR7 is a complex multispanner GPCR receptor activated by CCL19/CCL21 protein chemokines. CCR7 is necessary for Treg immune suppression. (Schneider M A et al. CCR7 is required for the in vivo function of CD4+ CD25+ regulatory T cells, Journal of Experimental Medicine Vol. 204, No. 4, Apr. 16, 2007 735-745); Lanzavecchia A, et al. Dynamics of T lymphocyte responses: intermediates, effectors, and memory cells. Science. 2000; 290:92-97 Sallusto F, et al. Understanding dendritic cell and T-lymphocyte traffic through the analysis of chemokine receptor expression. Immunol Rev. 2000; 177:134-140). Upon CCR7 inhibition or blockade, Treg immune suppression is arrested, anti-cancer immune activation will proceed through alternative pathways and anti-cancer immune responses accelerate.

Neutralizing, but not depleting, antibody blockade of CCR7 showed efficacy in a mouse melanoma model. Reversing immune tolerance of a CCL21 expressing melanoma; induces a strong anti-tumor immune response that reduced both Treg and tumor cell populations in melanoma, without inducing systemic autoimmune adverse events. (Shields, J. D. et al. (2010) Induction of lymphoid-like stroma and immune escape by tumors that express the chemokine CCL21 Science 2010 May 7; 328(5979):749-52).

### CCR7 Antagonism in Melanoma

According to Shields, J. D. et al. (2010) Induction of lymphoid-like stroma and immune escape by tumors that express the chemokine CCL21 Science 2010 May 7; 328(5979):749-52. neutralizing, but not depleting anti-CCR7 antibodies, and antibodies against CCL21 can be effective in tumor control equivalent to control animals (i.e., little tumor growth).

### CCR7 Rationale for Broader Efficacy of Anti-Tregs Cancer Immunotherapy

Clinical success can be achieved with antibody inhibition or blockade of a single aberrant cancer to immune cell signaling control only cancer induced mechanisms of immune suppression (e.g. CTLA-4 and PD-1) and does not control Treg induced pathways of immune suppression. Inhibiting or blocking cancer cell subversion of Tregs by CCR7 antibodies can arrest multiple CCR7⁺ Treg immune suppression mechanisms, and therefore offer broader efficacy. The survivability of the CCR7⁻/⁻ knockout mouse and the similarity of the autoimmune induction of the CCR7⁻/⁺ knock out mouse to the PD-1 knockout mouse together, means that CCR7 antibody inhibition or blockade will have a similar adverse event and side effect profiles to the PD-1 inhibitors.

Broad based use of cyclophosphamide in oncolytic chemotherapy to ablate Treg populations confirms that Treg blockade/ablation—either through cyclophosphamide and/or depleting antibodies is already a standard in cancer therapy (Ghiringhelli F et al. Metronomic cyclophosphamide regimen selectively depletes CD4+CD25+ regulatory T cells and restores T and NK effector functions in end stage cancer patients. Cancer Immunol Immunother 2007; 56:641-8). This leads to the following rationales:

1. CCR7 is required for Treg mediated immune suppression (Schneider et al., CCR7 is required for the in vivo function of CD4+ CD25+ regulatory T cells JEM Vol. 204, No. 4, Apr. 16, 2007 735-745).

2. CCR7 is highly expressed on Treg cells invasive of tumor micro-environments >90% of these Tregs are CCR7⁺ in many solid tumors.

3. CCR7 is not otherwise broadly expressed outside of immune system cells, making anti-CCR7 antibodies selective for Treg target cells in the body.

4. As a more selective, but more potent Treg suppressor, CCR7⁺ Treg inhibition or blockade can be a more efficacious and safer cancer therapy than cyclophosphamide or other, less selective, lymphocyte ablating antibodies like Rituxumab, Campath and daclizumab.

Treg Activation, Survival, and Apoptosis are CCR7 Dependent Functional CCR7 stimulation by CCL19 or CCL21 regulates chemotaxis and migratory speed (Rodriguez Fernández, J L., Molecular mechanisms that regulate the functions of the chemokine receptor CCR7 in dendritic cells; Riol-Blanco L The chemokine receptor CCR7 activates in dendritic cells two signaling modules that independently regulate chemotaxis and migratory speed. J Immunol. 2005 Apr. 1; 174(7):4070-80). Five mechanisms can be responsible for the effects of CCR7 in cancer.

(1) CCR7 stimulation activates the PIK3 AKT pathway;

(2) CCR7 inhibits or blocks the pro-apoptotic GSK3β FOXO1/3 pathway;

(3) CCR7 inhibition or blockade of IKK and thereby activates the NFκB pro-survival pathway,

(4) CCR7 stimulation can activate the ERK 1/2 JNK pathway that enables chemotaxis; and

(5) CCR7 stimulation can activate the Rho PYK2 Coffin pathway that can increase speed of chemotaxis.

The NF-kB signaling pathway is a key regulator of Foxp3 expression during natural Treg cell development and in Treg function (Long M, et al., Nuclear Factor-kB Modulates Regulatory T Cell Development by Directly Regulating Expression of Foxp3 Transcription Factor, Immunity 31, 921-931, Dec. 18, 2009).

Enhancing N-kB activity leads to increased number of Foxp3⁺ cells and can rescue Foxp3 expression in thymocytes deficient in other pleiotropic signaling molecules (Id.).

NF-kB directly promotes the transcription of Foxp3, and upon T cell receptor (TCR) stimulation, c-Rel, an NF-kB family member, bound to Foxp3 enhancer region, which is specifically demethylated in natural Treg cells (Zhou X et al. Plasticity of CD4+ FoxP3+ T cells Curr Opin Immunol. 2009 June; 21(3): 281-285; Zhou X et al., Foxp3 instability leads to the generation of pathogenic memory T cells in vivo. Nat Immunol. 2009; 10(9):1000-7) CCR7 inhibition or blockade can down regulate the NF-kB signaling pathway and should therefore down regulate Foxp3 expression.

CCR7 is required for the in viva function of CD4+ CD25+ regulatory T cells (Schneider M A et al. Journal of Experimental Medicine Vol. 204, No. 4, Apr. 16, 2007 735-745), CCR7 blockade can down regulate FoxP3 expression and thereby decreases Treg immunosuppression behavior.

CCR7 blockade can suppress Foxp3 expression in CCR7⁺ Tregs, CCR7 inhibition or blockade can arrest Treg activation and immunosuppression and mediate conversion of Treg immuno-suppressing phenotypes to the exTreg Th17 phenotype (Zhou X et al. Plasticity of CD4+ FoxP3+ T cells Curr Opin Immunol. 21(3): 281-285 (June 2009); Zhou X et al., Foxp3 instability leads to the generation of pathogenic memory T cells in vivo. Nat Immunol. 2009; 10(9):1000-7).

### Use of Fully Human Anti-CCR7 Antibodies to Treat Cancer

CCR7 is involved in a large number of cancers, making it a desirable target for immune therapy. Multiple indications can be pursued depending upon circumstances to provide the most direct route to alleviate suffering in patients with cancer. Potential indications include CLL, Refractory non-Hodgkins lymphoma, GvL/GvHD, metastatic melanoma, bladder cancer and many other CCR7⁺ solid tumors etc. Strategic options include complement activation, tumor cell depletion (acute disease) and overcoming immune tolerance for broad cancer therapy a la PD-1. Other indications that can be treated using CCR7 antibodies of this invention include cancers whose growth is regulated by FOS.

To use the antibodies of this invention to treat cancer, a patient presents with a diagnosis of cancer. After obtaining informed consent to treatment, the patient is treated using fully human anti-CCR7 antibodies of this invention. The antibodies are purified from cells that express the antibodies, and the antibodies are prepared in a delivery composition that is compatible with the patient and with the desired route of administration.

### Actions of Anti-CCR7 Antibodies

For some uses, it can be desirable to link an anti-CCR7 antibody to a reagent that acts to kill a cancer cell. For other uses, the anti-CCR7 antibodies can bind to the CCR7 receptors on the cancer cells, and either: (1) block binding of the CCR7 receptor native ligand (e.g., chemokines CCL 19 and CCL 21). Although the mechanisms responsible for the blocking effect are not completely known, several mechanisms have support. First, anti-CCR7 antibodies can bind to the extracellular domain of the CCR7 molecule, and can inhibit binding of the naturally occurring ligands CCL 19 and/or CCL 21. Alternatively, anti-CCR7 antibodies can bind to a portion of the CCR7 molecule and inhibit the effects of binding of a naturally occurring ligand. Still alternatively, binding of anti-CCR7 antibodies can lead to internalization of the antibody-CCR7 complex, thereby removing it from the cell's surface, and thereby lead to decreased function of CCR7. It is possible that other mechanisms could contribute to the therapeutic effect of anti-CCR7 antibodies, and all such mechanisms are considered to be part of this invention.

Thus, CCR7 antibodies of this invention can be sued to treat any cancer that utilizes the PIK3/AKT pathway, which blocks the pro-apoptotic GSK3β FOXO1/3 pathway and blocks IKK and thereby activates the NFκB pro-survival pathway. With inhibition of these pro-growth, anti-immune pathways, cancer can be effectively treated using antibodies of this invention.

For some uses, the composition can be injected into the circulation via a peripheral vein. For other uses, the compositions containing antibodies of this invention can be directly injected into a tumor (e.g., for a solid tumor). For other uses, compositions can be administered into a cerebral ventricle or into the cerebrospinal fluid. For other uses, antibodies of this invention can be delivered to the lungs by aerosol, to the upper airways (pharynx, trachea, nose) by instillation of liquid or gel, or to the skin by injection, salve, cream, or by high velocity micro-injection (e.g., “Powderject™” methods.

Anti-CCR7 depleting antibodies or ADCs (antibody-drug conjugates) can be used as an additional therapy, which can be safer than Declamizumab, Campath, Cyclophosphomide, and Rituximab.

Anti-CCR7 depleting MAB can broadly deplete lymphoid lineage cells (including central memory T cells). It will not deplete hematopoietic cells and will spare the innate immune system (e.g., myeloid lineage cells). Although a portion of the adaptive immune system might be compromised, the innate immune system of the patient can be preserved. This can provide immunity to infectious diseases like invasive Aspergillosis.

The adaptive immune system will rapidly regenerate.

Certain blood cancer patients can benefit from combined anti-CCR7 therapy (depletion and neutralization) for patients with late stage DLBCL, or patients with other CCR7+ blood cancers who will be subject of bone marrow (BM) transplantation procedures. Examples of such combination therapies are described below.

Step 1: Treat a patient with radio ablative therapy in combination with anti-CCR7 depleting antibody therapy.
Step 2: Bone marrow transplantation
Step 3: Maintain the patient on anti-CCR7 neutralizing antibody therapy. This can provide a triple therapeutic effect: (a) suppress Graft vs. Host Disease (GvHD), (b) accelerate proliferation of myeloid cells, which can lead to suppression of common fungal infections, and (c) break immune tolerance to cancer.

By this combined treatment procedure one can reduce the probability of primary cancer relapse.

### Safety of CCR7 Antibody Therapy

CCR7⁻/⁻ knockout animals are not immuno-compromised and maintain anti-infective responses (Hartigan A J, CCR7 impairs hematopoiesis following hematopoietic stem cell transplantation increasing susceptibility to invasive aspergillosis Blood. 2010 Dec. 9; 116(24):5383-93). Like PD-1⁻/⁻ knockout animals CCR7⁻/⁻ knockout animals are prone to auto-immune activation, but do not spontaneously develop auto-immune diseases (Förster R et al., CCR7 and its ligands: balancing immunity and tolerance Nature Reviews Immunology May 2008 volume 8 p. 365; Davalos-Misslitz A C, et al. (2007) Generalized multiorgan autoimmunity in CCR7− deficient mice. Eur J Immunol 37:613-622. 26). Like PD-1⁻/⁻ knockout animals CCR7⁻/⁻ knockout animals have heighted responses to diabetes or nephritis auto-immune challenge. (Id). Unlike FOXP3⁻/⁻ knock out animals or scurfy animals, CCR7⁻/⁻ knockout animals do not spontaneously develop IPEX, diabetes, pneumonitis, auto-immune nephritis. (Id).

As seen with Rituxumab anti-CD20 (depleting all B lymphoblasts and dendritic cells, but not T cells) Campath anti-CD52 (depleting all lymphoid myeloid lineage tissues except stem cells), anti-CD-25 daclizumab (depleting all IL-2 activated lymphocytes) and cyclophosphamide therapies, Treg suppression is central to many anti-cancer therapies and Treg reconstitution occurs rapidly after discontinuance of Treg ablation to avoid frank autoimmune reactions. Use of a neutralizing anti-CCR7 antibody will not pose the same level of risk as Treg and general CCR7⁺ cell depleting antibodies, but based upon CCR7 blockade of NFκB and FOXP3 cell signaling pathways may offer the prospect of arresting Treg mediated immune suppression on anti-cancer immune responses in Tregs without the need of full CCR7+ cell depletion.

### Induction of Cancer Cell Apoptosis and Arrest of Metastasis

Many metastatic cancers obtain a pro-survival benefit and chemotaxis function through expression of functional CCR7 (E. G., Wang J. et al. Autocrine and Paracrine Chemokine Receptor 7 Activation in Head and Neck Cancer: Implications for Therapy J Natl Cancer Inst 2008; 100: 502-512) (Takanami I. Overexpression of CCR7 mRNA in non-small cell lung cancer: correlation with lymph node metastasis. Int J Cancer. 2003; 105 (2): 186-189; Takeuchi H, et al. CCL21 chemokine regulates chemokine receptor CCR7 bearing malignant melanoma cells. Clin Cancer Res. 2004; 10 (7): 2351-2358 Pilkington K R, et al. Inhibition of generation of cytotoxic T lymphocyte activity by a CCL19/MIP-3beta antagonist. J Biol Chem. 2004; 279 (39): 40276-40282; Sanchez-Sanchez N, et al Chemokine receptor CCR7 induces intracellular signaling that inhibits apoptosis of mature dendritic cells. Blood. 2004; 104 (3): 619-625. Zhou Y., et al. CXCR4 is a major chemokine receptor on glioma cells).

CCR7 expression, which mediates immune cell survival and migration to lymph nodes, has recently been associated with nodal metastasis of squamous cell carcinoma of the head and neck (SCCHN) through activation of the pro-survival, PI3K/Akt pathway (Id.)

This survival pathway is constitutively activated in metastatic SCCHN cells and is enhanced by CCR7 ligand treatment. (Id.). In the absence of exogenous ligand, blocking CCR7 reduced the activation of phospho-Akt and Bcl2 in metastatic SCCHN cells, suggesting that secretion of CCR7 ligands, CCL19 and CCL21 (SLC) by tumor cells may be responsible for autocrine activation of CCR7. (Id.).

CCR7 blockade also decreased cell viability by MTT assay, and CCL19 induced-CCR7 activation protected metastatic SCCHN cells from cis-platinum induced apoptosis. Ibid. Pro-survival signals promote tumor progression of metastatic SCCHN cells, mediated through autocrine and paracrine CCR7 activation. (Id.).

The fact that expression of CCR7 and its ligands can propagate autocrine and paracrine survival signals, including constitutive PI3K-Akt pathway activation suggests that the CCR7 receptor may have potential as a novel therapeutic target.

The potential importance of NF-κB activation of CCR7 expression in certain CCR7+ cancers has been suggested by others, and we have obtained data that support this activation in our system.

The importance of inflammatory pathway mediators such as NF-κB or STAT-3 (in both immune and tumor cells) for which in vivo inhibitors are in clinical evaluation suggests that these signals should be studied in relation to CCR7 expression.

See also Arlt A et al. Role of NF-kappa B and Akt/PI3K n the resistance of pancreatic carcinoma cell lines against gemcitabine induced cell death. Oncogene. 2003; 22 (21): 3243-3251; Curiel T J, Wei S, Dong H, et al. Blockade of B7-H1 improves myeloid dendritic cell-mediated antitumor immunity. Nat Med. 2003; 9 (5): 562-567; Kane L P et al. Induction of NF-kappa B by the Akt/PKB kinase. Curr Biol. 1999; 9 (11): 601-604; Madrid L V, et al. Akt suppresses apoptosis by stimulating the transactivation potential of the RelA/p65 subunit of NF-kappa B. Mol Cell Biol. 2000; 20 (5): 1626-1638).

Blocking CCR7 on CCR7⁺ cancers arrest CCL19/CCL21/CCR7 mediated autocrine anti-apoptosis, pro-survival and chemotactic signaling mediated because CCR7 activation of PIK3/AKT blocks the pro-apoptotic GSK3β FOXO1/3 pathway and blocks IKK and thereby activates the NFκB pro-survival pathway. These effects make CCR7⁺ cancers more sensitive to pro-apoptosis signaling. CCR7 activation of ERK 1/2 JNK pathway enables chemotaxis, and CCR7 activation of The Rho PYK2 Coflin pathway enables higher chemotaxis speed. Therefore, use of anti-CCR7 antibodies: (1) produces apoptosis, (2) reduces immune suppression, and (3) decreases metastasis.

Treatment of Cancer

In certain embodiments, antibodies and/or fragments thereof of this invention can be used to treat cancers, including chronic leukocytic leukemia and other blood cancers, head and neck cancers, non-small cell lung cancers, gastric cancer, breast cancer, melanoma and colorectal cancer. In yet another embodiments, antibodies or fragment thereof conjugated with toxins or radionuclides can be used to treat these and other cancers, in which cells express CCR7. While anti-CCR7 antibody-based ADC is disclosed in the present invention, in general antibody-drug or antibody-radionuclide conjugates are well known to those skillful in the art. A number of contract research organizations perform such conjugation and drugs having ADC as an active component are on the market and are successfully used for treatment certain cancers. Each of these disorders, the roles of CCR7 and roles of anti-CCR7 antibodies are described further herein.

Chronic Leukocytic Leukemia and Other Blood Cancers

Chronic Leukocytic Leukemia (CLL) is one of the diseases where CCR7 molecules are overexpressed. Thus, use of binding or antagonist anti-CCR7 antibodies can be useful. The expression of CCR7 in other blood cancer cells has been also demonstrated for T-cell Acute Lymphoblastic Leukemia (T-ALL), Follicular Lymphoma (FL), and Mantle Cell Lymphoma (MCL) (Sonia Lopez-Giral et al., Chemokine receptors that mediate B cell homing to secondary lymphoid tissues are highly expressed in B cell chronic lymphocytic leukemia and non-Hodgkin lymphomas with widespread nodular dissemination., J. Leukoc. Biol. 76: 462-471; 2004; Anna Corcione et al., CCL19 and CXCL12 trigger in vitro chemotaxis of human mantle cell lymphoma B cells, Clinical Cancer Research, V. 10, 964-971, 2004).

Diffuse Large B Cell Lymphoma (DLBCL)

There are non-fully human antibodies against human CCR7. Some desirable properties of fully human anti-human CCR7 antibodies are shown in Table 1 below.

TABLE 1
Properties of Anti-CCR7 Antibodies

| | |
|---|---|
| Mechanisms of Action: | Direct depletion of CCR7 cancer cells |
| | Braking immune tolerance and metastasis by CCR7 neutralization |
| | Anti-Graft vs. Host Disease/pro Graft vs. Leukemia (GVL) in HSCT. |
| Required MAb features: | Neutralize CCR7 ligand signaling |
| | MAb internalization desirable for cell depleting ADC and CCR7 |
| | desensitization |
| Format: | Human IgG4 and ADC |
| Affinity (EC₅₀) | about 1 nM |
| Functionality: | CCL19 neutralizing, Ca⁺⁺ flux IC₅₀ about 25 nM, internalizing |

| Treatment Protocol | | Treatment Effect |
|---|---|---|
| Step 1: | Injection of anti-CCR7 cell depleting MAb | 1) Depletion of CCR7+ cancer cells |
| | (IgG1 or ADC) | 2) Depletion of host T+ and B Cell subsets |
| | | and antigen presenting cells (anti-GVHD) |
| Step 2: | Radiotherapy/HSCT | Combination Therapy |
| Step 3: | Injection of anti-CCR7 neutralizing MAb | 3) Brake immune tolerance |
| | (IgG4) | 4) Anti-GVHD |
| | | 5) GVL |
| | | 6) promote anti-fungal immunity |

### Comparison of Prior Art Anti-CCR7 Antibodies

By way of comparison, prior art anti-CCR7 antibodies may not be suitable for use in human beings for therapeutic purposes. In particular, mouse and rat antibodies have been developed, yet do not meet the criteria desirable for use in human beings (see Table 2).

TABLE 2
Prior Art Anti-CCR7 Antibodies

| Mouse Anti Human CCR7 MAb | Rat Anti Mouse CCR7 MAb |
|---|---|
| Mouse MAb IgG2A (R&D) | Rat IgG2A (R&D) |
| Cross reacts with human CCR7 | Reacts with mouse CCR7 |
| No cross-reactivity with Mouse | No cross-reactivity with human |
| CCR7 | CCR7 |
| Neutralizes human CCL19-induced | Neutralizes mouse CCL19 |
| chemotaxis IC₅₀ about 15 nM | (IC₅₀ about 40 nM |

Head and Neck Cancer

Head and neck carcinomas are histologically and clinically heterogeneous. While squamous cell carcinomas (SCC) are characterized by lymphogenous spread, adenoid cystic carcinomas (ACC) disseminate preferentially hematogenously. Analysis at the mRNA and protein level of human chemokine receptors showed that SCC and ACC cells exhibited distinct and nonrandom expression profiles for these receptors. SCC predominantly expressed receptors for chemokines homeostatically expressed in lymph nodes, including CC chemokine receptor CCR7.

CCR7 mediates survival and invasiveness of metastatic squamous cell carcinoma of the head and neck (SCCHN) to regional lymph nodes. According to Wang et al., Autocrine and paracrine chemokine receptor 7 activation in head and neck cancer: implications for therapy. J. Natl. Cancer Inst., 100: 502-512. (2008), constitutive pro-survival signaling by the phosphoinositide-3 kinase/Akt pathway has been observed in SCCHN cells independent of epidermal growth factor receptor (EGFR) signaling. Expression and secretion of chemokines by primary tumors, metastatic nodes, and benign nodes of patients with SCCHN were determined by quantitative real-time polymerase chain reaction and enzyme-linked immunosorbent assay, respectively (Wang et al. Id.). The role of paracrine activation of CCR7 on tumor growth was analyzed by comparing the growth of orthotopic tumors derived from B7E3 murine oral carcinoma cells in wild-type BALB/c mice, in paucity of lymphoid T cell (plt, deficient in CCL19 and CCL21 expression) mice, and in pit mice in which the implanted B7E3 cells overexpressed CCR7 (Id.). In the absence of exogenous ligand treatment, blockade of CCR7 signaling reduced levels of phosphorylated (activated) Akt and decreased SCCHN cell-viability by up to 59%, enhancing the effect of EGFR inhibition (Id.). CCR7 stimulation protected metastatic SCCHN cells from cisplatin-induced apoptosis in an Akt-dependent manner (Id.). Metastatic nodes expressed and secreted higher levels of CCL19 than benign nodes or primary tumors. Secretion of CCL19 and CCL21 by SCCHN cells and by paracrine sources combine to promote activation of CCR7 pro-survival signaling associated with tumor progression and disease relapse. CCR7 and its cognate chemokines may be useful biomarkers of SCCHN progression, and blockade of CCR7-mediated signaling may enhance the efficacy of platinum- and EGFR-based therapies. (Id.).

In treating patients with head and neck cancer, after a diagnosis is made, the patient is treated with the anti-CCR7 antibody or fragment of this invention until one or more characteristic signs and/or clinical findings indicate that therapy has been at least partially successful.

It can also be appreciated that anti-CCR7 antibodies of this invention can be used for diagnosing or evaluating the CCR7 status of a cell or tissue.

Non-Small Cell Lung Cancer

Tumor cell migration into the lymph nodes is an important aspect of cancer and CCR7 has been shown to play an important role in tumor cell migration and lymph node metastasis. Takanami, I. Overexpression of CCR7 mRNA in nonsmall cell lung cancer: correlation with lymph node metastasis. Int. J. Cancer 2003 Jun. 10; 105(2):186-189 investigated CCR7 expression in 71 patients with NSCLC who underwent curative tumor resection and found that CCR7 mRNA was expressed in 45 cases (63.3%; Takanami, Id.). The CCR7 mRNA expression was significantly associated with lymph node metastasis, stage, lymphatic invasion. Twenty-six (57.8%) of 45 cases with CCR7 mRNA expression in their cancer tissues were node-positive, whereas only 3 (11.5%) of 26 cases without CCR7 mRNA expression were node-positive. Furthermore, expression of CCR7 mRNA was shown to be an independent predictor of lymph node metastasis by multivariate analysis (p=0.0117). Our study demonstrates that CCR7 might be related to the development of lymph node metastasis in NSCLC. The expression of CCR7 mRNA could open up a new window for the diagnostic staging and treatment of NSCLC (Id.).

Expression of CCR7 in pulmonary tumor tissues and metastasized lymph nodes in NSCLC has been measured in specimens from 17 cases of adenocarcinoma, 17 cases of Squamous cell Carcinoma, 12 cases of Adenosquamous Carcinoma, 4 cases of large cell carcinoma and 28 cases of metastasized lymph nodes of lung cancer (Zeng, T., Wen, J. The value and association of CCR7 expression in NSCLC with lymph node metastasis. Chinese Journal of Lung Cancer, 11: No 2 (2008)). The expression of CCR7 in pulmonary tumor tissue was remarkably higher than normal lung tissue (Id.).

In treating patients with non-small cell lung cancer, after a diagnosis is made, the patient is treated with the anti-CCR7 antibody or fragment of this invention until the characteristic signs and/or clinical findings indicate that therapy has been at least partially successful.

Gastric Cancer and Pancreatic Cancer

Chemokine receptor CCR7 is a key molecule for migration of lymphocytes and dendritic cells into lymph nodes (Ishigami et al., Prognostic value of CCR7 expression in gastric cancer. Hepatogastroenterology 54:1025-1028 (2007)). Expression of CCR7 in tumor cells has been reported in malignancies, and CCR7 expression in tumor cells has been investigated in vitro and in vivo. A total of 224 gastric cancer patients who underwent curative surgery were enrolled and CCR7 expression in the primary tumor was detected. Patients showing more than 10% positivity for CCR7 were defined as having high CCR7 expression, as previously reported. CCR7 expression was detected in tumor cells and inflammatory cells in the tumor nest. CCR7-positive patients exhibited deeper tumor invasion, more frequent lymph node metastasis, higher rates of lymphatic invasion and more venous invasion than CCR7-negative patients. Most significant clinical factor for CCR7 was lymph node metastasis followed by lymphatic invasion. CCR7-positive gastric cancer patients had significantly poorer surgical outcomes than CCR7-negative patients. Our results suggest that CCR7 expression in gastric cancer is related to the onset of preferential conditions for lymphatic spread, such as lymph node metastasis. CCR7 expression of preoperative biopsy specimen can predict lymph node metastasis.

In treating patients with gastric cancer or pancreatic cancer, after a diagnosis is made, the patient is treated with the anti-CCR7 antibody or fragment of this invention until the characteristic signs and/or clinical findings indicate that therapy has been at least partially successful.

Breast Cancer

Recent studies have indicated that expression of chemokine receptors CXCR4 and CCR7 could be an indicator of the metastatic potential of breast cancer (Cabioglu N. et al., Expression of growth factor and chemokine receptors: new insights in the biology of inflammatory breast cancer. Ann Oncol. 2007 June; 18(6):1021-9). Expression of CXCR4 and CCR7 along with the biomarkers HER2-neu and epidermal growth factor receptor (EGFR) was investigated in inflammatory breast cancer (IBC) to evaluate their prognostic implications (Cabioglu N. et al. Id.). CXCR4, CCR7, and EGFR were evaluated by immunohistochemical staining (IHC) of paraffin-embedded tissue sections. HER2-neu amplification was assessed by FISH and/or IHC. All patients received chemotherapy, surgery, and radiation (Id.). Forty-four cases diagnosed with IBC from 1994 to 2002 were included in the study. In all, 18 (40.9%) patients had positive CXCR4, 10 (22.7%) had positive CCR7, 21 (47.7%) had positive HER2-neu, and EGFR was positive in 12 of 40 patients (30%). The 5-year overall survival (OS) was 24.8% for CXCR4-positive disease versus 42.3% for CXCR4-negative patients (P=0.53) and 20.0% for CCR7-positive disease versus 41.9% for CCR7-negative patients (P=0.24). EGFR-positive disease had significantly worse OS compared with EGFR-negative disease (P=0.01). These data demonstrate the roles of expression of growth factor and chemokine in pathogenesis of this disease.

Animal Studies are Predictive of Human Efficacy

Among mouse models that can be used to demonstrate the efficacy of anti-CCR7 antibodies are, for example, a murine transplantation model of atherosclerosis regression as described in Feig J E, Quick J S, and Fisher E A, The role of a murine transplantation model of atherosclerosis regression in drug discovery. Curr Opin Investig Drugs. 2009 March; 10(3):232-8. incorporated herein fully by reference. According to the authors, “a transplantation-based mouse model of atherosclerosis regression has been developed by allowing plaques to form in a model of human atherosclerosis, the apoE-deficient mouse, and then placing these plaques into recipient mice with a normolipidemic plasma environment. Under these conditions, the depletion of foam cells occurs. Interestingly, the disappearance of foam cells was primarily due to migration in a CCR7-dependent manner to regional and systemic lymph nodes after 3 days in the normolipidemic (regression) environment.” Thus, this model can be useful in determining the effect on anti-CCR7 antibodies on the disease progression.

Several other well-established models can be used for development of anti-cancer drugs based upon anti-CCR7 antibodies of this invention. For example, such as experimental mouse model described in Kochetkova M, Kumar S, McColl S R, Chemokine receptors CXCR4 and CCR7 promote metastasis by preventing anoikis in cancer cells. Cell Death Differ. 2009 May; 16(5):664-73. Epub 2009 Jan. 9 incorporated herein fully by reference. This model was used to uncover “a novel property of the chemokine receptors CXCR4 and CCR7 in inhibiting detachment-induced cell death—anoikis, which is believed to be one of the major blocks in the metastatic spread of various neoplasms.” The results obtained provide evidence for a previously unknown axis in malignant tumors, which connects chemokine receptors with deregulated apoptosis and relates to metastatic breast and potentially other tumors.

Another model that links CCR7 and cancer is described in Yu S, Duan J, Zhou Z, Pang Q, Wuyang J, Liu T, He X, Xinfa L, Chen Y, A critical role of CCR7 in invasiveness and metastasis of SW620 colon cancer cell in vitro and in vivo. Cancer Biol Ther. 2008 July; 7(7):1037-43. Epub 2008 Apr. 7 that is incorporated herein fully by reference. The authors employed RNA interference to detect the in vitro effects of anti-CCR7 siRNAs on proliferation and invasiveness of SW620 cells and evaluated the ability of these siRNAs to inhibit the lymphogenesis and the lymph node metastasis in xenografted SW620 tumors in mice. In this animal model, blocking CCR7 expression at the mRNA level impaired invasion of colon cancer cells and inhibited lymph node metastasis of colon cancer and lymphogenesis.

Yet another cancer-related model that can be applied for the exploration of CCR7 antibodies of this invention is provided in Koizumi K, Kozawa Y, Ohashi Y, Nakamura E S, Aozuka Y, Sakurai H, Ichiki K, Doki Y, Misaki T, Saiki I., CCL21 promotes the migration and adhesion of highly lymph node metastatic human non-small cell lung cancer Lu-99 in vitro. Oncol Rep. 2007 June; 17(6):1511-6 (incorporated herein in full by reference). According to this article, “[t]o develop new therapy strategies for lung cancer, we established an animal model, which reflects the clinical features of mediastinal lymph node metastasis of lung cancer. This study was designed to determine whether CCL21 induced biological functions associated with the metastasis of highly lymph node metastatic human non-small cell lung cancer (NSCLC) selected by our model. Orthotopic intrapulmonary implantation of human NSCLC (Lu-99 and A549) was performed to analyze the metastatic characteristics of these cells. The expression of CCR7, which is a receptor of CCL21, was detected using CCL19 [also called EBI1-ligand chemokine (ELC)]-Fc chimera by flow cytometric analysis. The effects of CCL21 on the migration, adhesion and growth of human NSCLC were investigated. After orthotopic implantation of human NSCLC cell lines, Lu-99, but not A549, metastasized to mediastinal lymph nodes, forming large size nodules, and expressed CCR7 on the surface. Accordingly, its ligand CCL21 induced chemotactic migration and alpha4beta1-mediated adhesion to VCAM-1 of Lu-99. The expression of CCR7 and vigorous responses to its ligand CCL21 potentially account for lymph node metastasis of a human NSCLC line Lu-99.”

Another cancer-related model that can be applied for the exploration of fully human CCR7 antibodies of this invention is provided in Wang J, Xi L, Hunt J L, Gooding W, Whiteside T L, Chen Z, Godfrey T E, Ferris R L., Expression pattern of chemokine receptor 6 (CCR6) and CCR7 in squamous cell carcinoma of the head and neck identifies a novel metastatic phenotype. Cancer Res. 2004 Mar. 1; 64(5):1861-6., incorporated herein fully by reference. According to this paper, “squamous cell carcinoma of the head and neck (SCCHN) metastasizes predictably to cervical lymph nodes, with low rates of distant metastases. Tumor cells can express various receptors that facilitate such metastatic spread to lymph nodes and other non-lymphoid organs. Chemokine receptors (CCR), normally expressed on lymphocytes, control immune and inflammatory cell migration, providing a link between innate and adaptive immunity Chemokine receptor expression was evaluated in SCCHN, using paired primary and metastatic tumors cell lines, and paired primary and metastatic biopsies from the same patients. Quantitative reverse transcription-PCR showed a consistent pattern of CCR6 down-regulation and up-regulation of CCR7 in metastatic cells and tissues. Chemotaxis assays, ligand-induced receptor down-regulation, and specific antibody blocking experiments supported the quantitative reverse transcription-PCR results, indicating that these surface receptors were functional on metastatic tumor cells. Cells derived from a highly metastatic mouse model of SCCHN were used to confirm CCR7 up-regulation in tumor cells with higher metastatic potential. CCR6 down-regulation is consistent with its decreased expression in cells emigrating from peripheral mucosal sites, whereas CCR7, important for homing of immune cells to secondary lymphoid organs, was significantly up-regulated. Thus, CCR6, CCR7, and their ligands, normally important in controlling immune cell trafficking in response to inflammatory stimuli, may have an important role in determining the metastasis of SCCHN cells in vivo.”

Yet another cancer-related use of the anti-CCR7 antibodies of this invention can be explored as described in Arenberg D A, Zlotnick A, Strom S R, Burdick M D, Strieter R M., The murine CC chemokine, 6C-kine, inhibits tumor growth and angiogenesis in a human lung cancer SCID mouse model. Cancer Immunol Immunother. 2001 January; 49(11):587-92, incorporated herein fully by reference. In this study tumor growth in severe combined immunodeficiency (SCID) mice was linked to interaction of CCR7 ligand with murine 6C-kine binding to one of the CXC chemokine receptors CXCR3, in addition to its other known receptor CCR7.

A further cancer model useful for studying anti-CCR7 antibodies can be applied as described in Saur D, Seidler B, Schneider G, Algül H, Beck R, Senekowitsch-Schmidtke R, Schwaiger M, Schmid R M., CXCR4 expression increases liver and lung metastasis in a mouse model of pancreatic cancer. Gastroenterology. 2005 October; 129(4); 1237-50, incorporated herein fully by reference. That study utilized noninvasive imaging of targeted metastasis in a mouse model of pancreatic cancer; functional expression of the chemokine receptors CXCR4 and CCR7 was achieved by stable transfection of murine TD-2 pancreatic cancer cells and analyzed by flow cytometry, calcium flux, migration, and proliferation assays. The metastatic potential of the different stable TD-2 cell clones was assessed by tail vein metastatic assays in nude mice using in vivo bioluminescent imaging.

Another cancer-related model that can be used to obtain support for the use of anti-CCR7 antibodies of this invention of the treatment of cancer is described in Murakami T, Cardones A R, Hwang S T., Chemokine receptors and melanoma metastasis. J Dermatol Sci. 2004 November; 36(2):71-8 (incorporated herein fully by reference). According to this article, “[c]ancer metastasis is the end result of a complex series of biologic events that leads to the formation of clinically significant secondary tumors at distant sites. The sites of distant metastasis are not random since certain tumors show a tendency to develop metastases in specific organs. Human melanoma, for example, demonstrates frequent metastasis to brain, lungs, lymph nodes, and skin. Herein, we review the evidence that suggests that a limited number of chemokine receptors may play critical roles in determining organ-selective metastasis in melanoma by regulating diverse processes such as chemoattraction, adhesion, and survival. In particular, we describe roles for CC chemokine receptor 7 (CCR7) in lymph node metastasis . . . using a mouse model of melanoma. Preliminary evidence in this preclinical model suggests that inhibiting the function of these receptors may decrease the ability of cancer cells to disseminate to other sites and/or block their ability to survive and form tumors.”

Other models can be used by those skillful in the art to explore the use of anti-human CCR7 antibodies of this invention in inflammatory treatment, similarly to that described for example in Xia M, Hou C, DeMong D E, Pollack S R, Pan M, Brackley J A, Jain N, Gerchak C, Singer M, Malaviya R, Matheis M, Olini G, Cavender D, Wachter M., Synthesis, structure-activity relationship and in vivo antiinflammatory efficacy of substituted dipiperidines as CCR2 antagonists. J Med Chem. 2007 Nov. 15; 50(23):5561-3. Epub 2007 Oct. 11 (incorporated by reference in full) for a series of substituted dipiperidine compounds synthesized and identified as selective CCR2 antagonists, some had outstanding selectivity over CCR1, CCR3, CCR4, CCR5, CCR6, CCR7, and CCR8 and showed excellent efficacy in adjuvant-induced arthritis model, collagen-induced arthritis model, and allergic asthma model.

Yet another model is as in Wengner A M, Höpken U E, Petrow P K, Hartmann S, Schurigt U, Bräuer R, Lipp M., CXCR5- and CCR7-dependent lymphoid neogenesis in a murine model of chronic antigen-induced arthritis. Arthritis Rheum. 2007 October; 56(10):3271-83, by (incorporated fully by reference). This study has established a murine model of chronic arthritis in which the development of tertiary lymphoid tissue, a hallmark of human rheumatoid arthritis, is locally induced. The role of the homeostatic chemokine receptor CCR7 in this process can be explored using the model of chronic antigen-induced arthritis in mice with a strong bias toward inflammation, as described therein.

Yet another model for exploring the use of anti-human CCR7 antibodies of this invention is described in Pierce E M, Carpenter K, Jakubzick C, Kunkel S L, Flaherty K R, Martinez F J, Hogaboam C M., Therapeutic targeting of CC ligand 21 or CC chemokine receptor 7 abrogates pulmonary fibrosis induced by the adoptive transfer of human pulmonary, fibroblasts to immunodeficient mice. Am J Pathol. 2007 April; 170(4):1152-64 (incorporated herein fully by reference). According to this article, “[i]diopathic interstitial pneumonias (IIPs) are a collection of pulmonary fibrotic diseases of unknown etiopathogenesis. CC chemokine receptor 7 (CCR7) is expressed in IIP biopsies and primary fibroblast lines, but its role in pulmonary fibrosis was not previously examined. To study the in vivo role of CCR7 in a novel model of pulmonary fibrosis, 1.0×10(6) primary fibroblasts grown from idiopathic pulmonary fibrosis/usual interstitial pneumonia, nonspecific interstitial pneumonia, or histologically normal biopsies were injected intravenously into C.B-17 severe combined immunodeficiency (SCID)/beige (bg) mice. At days 35 and 63 after idiopathic pulmonary fibrosis/usual interstitial pneumonia fibroblast injection, patchy interstitial fibrosis and increased hydroxyproline were present in the lungs of immunodeficient mice. Adoptively transferred nonspecific interstitial pneumonia fibroblasts caused a more diffuse interstitial fibrosis and increased hydroxyproline levels at both times, but injected normal human fibroblasts did not induce interstitial remodeling changes in C.B-17SCID/bg mice. Systemic therapeutic immunoneutralization of either human CCR7 or CC ligand 21, its ligand, significantly attenuated the pulmonary fibrosis in groups of C.B-17SCID/bg mice that received either type of IIP fibroblasts. Thus, the present study demonstrates that pulmonary fibrosis is initiated by the intravenous introduction of primary human fibroblast lines into immunodeficient mice, and this fibrotic response is dependent on the interaction between CC ligand 21 and CCR7.

And yet another use of anti-CCR7 antibodies of this invention can be exploited as described in Yamashita N, Tashimo H, Matsuo Y, Ishida H, Yoshiura K, Sato K, Yamashita N, Kakiuchi T, Ohta K., Role of CCL21 and CCL19 in allergic inflammation in the ovalbumin-specific murine asthmatic model. J Allergy Clin Immunol. 2006 May; 117(5):1040-6 (incorporated herein fully by reference. According to this article, dendritic cells are the most powerful of the antigen-presenting cells and are known to play important roles in sensitization and inflammation in allergen-specific asthma. The role of CCL21 in airway inflammation in asthma was explored by using BALB/c-plt/plt (plt) mice, which possess genetic defects in expression of both CCL21 and CCL19. Chemokine ligand (CCL)21, a key chemokine in the entry of naive T cells and antigen-stimulated dendritic cells into the T-cell zones of secondary lymphoid organs, which is a critical process in antigen-specific T-cell activation. Pit and control BALB/c mice were immunized with ovalbumin and alum 4 times and thereafter were subjected to a 2-week regimen of ovalbumin inhalation. In plt mice, ovalbumin-specific IgE response was delayed compared with control BALB/c mice, but they had the same level of response after final immunization. Although airway inflammation and response to acetylcholine were significantly reduced compared with BALB/c mice, significant eosinophilic inflammation and hyperresponsiveness were also observed in pit mice after 2 weeks of inhalation. Four weeks after cessation of inhalation, airway inflammation and hyperresponsiveness in pit mice were greater than in BALB/c mice. At the time of resolution of airway inflammation, IL-10 production was enhanced in BALB/c mice but not in plt mice. The chemokines CCL21 and CCL19 were critical for resolution of airway inflammation. The findings about the chemokines for induction and resolution of inflammation are key to establishing a new strategy for asthma immunotherapy.

Another use of the antibodies of this invention can be in stem cell treatment. Sordi V, Malosio M L, Marchesi F, Mercalli A, Melzi R, Giordano T, Belmonte N, Ferrari G, Leone B E, Bertuzzi F, Zerbini G, Allavena P, Bonifacio E, Piemonti L., Bone marrow mesenchymal stem cells express a restricted set of functionally active chemokine receptors capable of promoting migration to pancreatic islets. Blood. 2005 Jul. 15; 106(2):419-27. Epub 2005 Mar. 22 incorporated herein fully by reference. According to this article, “[b]one marrow-derived mesenchymal stem cells (BM-MSCs) are stromal cells with the ability to proliferate and differentiate into many tissues. Although they represent powerful tools for several therapeutic settings, mechanisms regulating their migration to peripheral tissues are still unknown. Here, we report chemokine receptor expression on human BM-MSCs and their role in mediating migration to tissues. A minority of BM-MSCs (2% to 25%) expressed a restricted set of chemokine receptors (CXC receptor 4 [CXCR4], CX3C receptor 1 [CX3CR1], CXCR6, CC chemokine receptor 1 [CCR1], CCR7) and, accordingly, showed appreciable chemotactic migration in response to the chemokines CXC ligand 12 (CXCL12), CX3CL1, CXCL16, CC chemokine ligand 3 (CCL3), and CCL19. Using human pancreatic islets as an in vitro model of peripheral tissue, we showed that islet supernatants released factors able to attract BM-MSCs in vitro, and this attraction was principally mediated by CX3CL1 and CXCL12. Moreover, cells with features of BM-MSCs were detected within the pancreatic islets of mice injected with green fluorescent protein (GFP)-positive BM. A population of bona fide MSCs that also expressed CXCR4, CXCR6, CCR1, and CCR7 could be isolated from normal adult human pancreas. This study defines the chemokine receptor repertoire of human BM-MSCs that determines their migratory activity. Modulation of homing capacity may be instrumental for harnessing the therapeutic potential of BM-MSCs.”

Yet another use of the anti-CCR7 antibodies of this invention can be developed by those skillful in the art using models and approaches described in Martin A P, Coronel E C, Sano G, Cheri S C, Vassileva G, Canasto-Chibuque C, Sedgwick J D., Frenette P S, Lipp M, Furtado G C, Lira S A., A novel model for lymphocytic infiltration of the thyroid gland generated by transgenic expression of the CC chemokine CCL21, J Immunol. 2004 Oct. 15; 173(8):4791-8, incorporated herein fully by reference. According to this article, “[l]ymphocytic infiltrates and lymphoid follicles with germinal centers are often detected in autoimmune thyroid disease (AITD), but the mechanisms underlying lymphocyte entry and organization in the thyroid remain unknown. We tested the hypothesis that CCL21, a chemokine that regulates homeostatic lymphocyte trafficking, and whose expression has been detected in AITD, is involved in the migration of lymphocytes to the thyroid. We show that transgenic mice expressing CCL21 from the thyroglobulin promoter (TGCCL21 mice) have significant lymphocytic infiltrates, which are topologically segregated into B and T cell areas. Although high endothelial venules expressing peripheral lymph node addressin were frequently observed in the thyroid tissue, lymphocyte recruitment was independent of L-selectin or lymphotoxin-alpha but required CCR7 expression. Taken together, these results indicate that CCL21 is sufficient to drive lymphocyte recruitment to the thyroid, suggest that CCL21 is involved in AITD pathogenesis, and establish TGCCL21 transgenic mice as a novel model to study the formation and function of lymphoid follicles in the thyroid.”

While those skillful in the art can suggest to apply the antibodies of this invention to many other treatments, we provide an addition model among these many, that can be used as described in Hopken U E, Droese J, Li J P, Joergensen J, Breitfeld D, Zerwes H G, Lipp M., The chemokine receptor CCR7 controls lymph node-dependent cytotoxic T cell priming in alloimmune responses. Eur J Immunol. 2004 February; 34(2):461-70, incorporated herein fully by reference. According to this article, “[t]he chemokine receptor CCR7 and its ligands regulate migration and co-localization of T cells and mature dendritic cells to and within secondary lymphoid organs. The requirement of CCR7 in efficient priming of allospecific cytotoxic CD8(+) T cells is poorly characterized. Here, we demonstrate a role for CCR7 in the initiation of an alloimmune response and in the development of transplant rejection. Remarkably, in a model of acute allogeneic tumor rejection, CCR7(−/−) mice completely failed to reject subcutaneously injected MHC class I mismatched tumor cells and cytotoxic activity of allospecific T cells was severely compromised. When solid tumors derived from wild-type mice were transplanted, recipient CCR7(−/−) mice were capable of rejecting the allografts. In contrast, tumor allografts transplanted from CCR7(−/−) donors onto CCR7(−/−) recipients showed allograft survival up to 28 days, suggesting a critical function of CCR7 on donor-type passenger leukocytes in the initiation of cytotoxic CD8(+) T cell responses. In a heterotopic heart transplantation model CCR7 deficiency resulted in significantly prolonged but not indefinite allograft survival. Additional prolongation of graft survival was observed when hearts from CCR7(−/−) mice were used as donor organs. Our results define a key role for CCR7 in allogeneic T cell priming within the context of draining lymph nodes.”

### Treatment of Multiple Sclerosis

Multiple Sclerosis is a complex, debilitating disease in which a number of immune system cells, such as CD8 positive effector T cells, central memory T cells, B-cells and dendritic cells (DCs) are implicated. Recent successes in clinical studies of the use of Rituxumab anti-CD20 (depleting all B lymphoblasts and dendritic cells, but not T cells) and Campath (Lemtrada) anti-CD52 (depleting all lymphoid myeloid lineage tissues except stem cells) for treatment of Multiple Sclerosis (MS) indicate that depletion of certain populations of blood cells can be advantageous for MS patients. As CCR7 is expressed on a number of cell types that can be affected by treatments with anti-CD20 or anti-CD52 depleting antibodies, the anti-CCR7 antibodies of this invention can be used to treat MS. Because various cells can be affected by fully human anti-CCR7 antibodies of this invention (either depleting antibodies or neutralizing antibodies), or a combination of these antibodies, such therapies are also embodiments of this invention. Further, use of antibodies of this invention can be advantageous compared to Compath and Rituximab: CCR7+ types of cells are much more narrow as compared to those affected by Campath, and thus there will be fewer undesirable side effects to be experienced by an MS patient. As compared to Rituximab that mostly affects B-cell population, anti-CCR7 antibodies of this invention can affect both B-cells and other implicated in the disease CCR7+ cells (T-cells, DCs) thus providing better efficacy in MS treatment.

### Physicochemical Properties of Monoclonal Antibodies Against Human CCR7

For a diagnostic, prognostic, and/or therapeutic use of antibodies, it can be desirable that the antibodies have physicochemical properties suitable for manufacture, formulation, packaging, and storage. Therefore, antibodies of this invention can be easily manufactured, using methods known in the art. We also found that antibodies of this invention are stable in the face of changes in temperature, are resistant to protease degradation, and do not undesirably degrade with time.

### Modifications to Antibodies to CCR7 I

In addition to the specifically identified antibodies shown in Tables 2-9, variations of these sequences can also be used. Conservative substitution of certain amino acids does not adversely affect binding of antibodies to their targets. Such conservative substitutions are shown below in Table 3.

TABLE 3
Conservative Amino Acid Substitutions

| Feature | Substituting Amino Acids |
|---|---|
| Basic side chains: | arginine, histidine, lysine |
| Acidic side chains: | aspartic acid, glutamic acid |
| Uncharged polar side chains: | asparagine, cysteine, glutamine, glycine, |
| | serine, threonine, tyrosine, tryptophan |
| Nonpolar side chains: | alanine, isoleucine, leucine, phenylalanine, |
| | proline, methionine, valine |
| Branched side chains: | isoleucine, threonine, valine |
| Aromatic side chains: | histidine, tyrosine, phenalalanine, tryptophan |

In addition to the above conservative amino acid substitutions, other variants of anti-CCR7 antibodies have been produced using site-directed mutagenesis. We have identified numerous variant CDR3 sequences, that when in antibodies of this invention bind to human CCR7. Such variants can also be effective in treating disorders characterized by either over expression of CCR7, or by over-stimulation of CCR7-expressing cells by chemokines (e.g., CCL19 and CCL21).

Compositions Containing Human Monoclonal Antibodies of this Invention

Antibodies of this invention can be formulated in a variety of ways to produce liquid solutions, suspensions, packaged into liposomes, attached to beads, or other types of compositions for therapeutic uses. For example, antibodies of this invention can be placed in a physiologically compatible solvent (e.g., phosphate buffered saline having physiologically compatible osmotic pressure, etc.). Additionally, antibodies may be formulated with other agents, including lipids, detergents, solubilizing agents, or other materials.

### Formulations

It can be desirable to inhibit aggregation of antibodies in solution. To do this, we use formulations containing an acidic buffer to stay away from the pI of an antibody in question. By protonating the antibodies, the acquire net positive charge, which produces an electrostatic repulsion, thereby keeping the antibodies from aggregating. For example, a pH of 5.8 can be used to inhibit aggregation. Suitable buffers include His-HCl, Na Citrate, Phosphate-Citrate, and Na Acetate.

It can also be desirable to avoid use of salt solutions, such as NaCl, because salts may decrease the effectiveness of low pH, and thereby may diminish the anti-aggregation properties of the acidic buffer used. Therefore, in some embodiments, NaCl seems can be omitted from the media, even for IV preparations.

It can also be useful to use polysorbate 20 (Tween 20™) or polysorbate 80 (Tween 80™). In some embodiments, one can use up to 0.5% by volume for IV preparations.

In other embodiments, one can include a sugar and or sugar alcohol. In some of these embodiments, one can use concentrations in the range up to 5% wt/vol., and in other embodiments, and up 10% wt/vol Sugars can stabilize lyophilized Abs by inhibiting protein denaturation upon drying and dissolving. Exemplary sugars include α,α-trehalose, sucrose, maltose, and sugar alcohols including mannitol or sorbitol.

IgG1 antibodies can be formulated containing about ˜5 mg/mL in a 10 mM acidic buffer, pH 5.5-5.8. Other agents, including Tween™, sugars, sugar alcohols, and other agents.

Other formulations that can be used include those listed below in Table 4. It can be appreciated that the above or other formulations can be used with anti-CCR7 antibodies of this invention.

TABLE 4
Formulations for Antibody Drugs

| | | | Powder | Stock | | | | |
|---|---|---|---|---|---|---|---|---|
| Commerc. | | Route of | or | Conc. | | | NaCl | |
| Name | Type | Admin. | Solution | mg/mL | pH | Buffer | mg/mL | Other Components/ |
| ABThrax | Anti-B. | | | | | | | |
| | anthrasis PA; | | | | | | | |
| | Human IgG1 | | | | | | | |
| Actemra | Anti-IL6R; | IV | Solution. | 20 | ~6.5 | Na | 0 | Disodium phosphate |
| | Humanized | | | | | Phos. | | dodecahydrate and sodium |
| | IgG1 | | | | | | | dihydrogen phosphate |
| | | | | | | | | dehydrate (as a 15 mM |
| | | | | | | | | phosphate buffer), |
| | | | | | | | | polysorbate 80 (0.5 |
| | | | | | | | | mg/mL), and sucrose (50 |
| | | | | | | | | mg/mL). |
| Numax | Anti-RSV; | | | | | | | |
| | Humanized | | | | | | | |
| | IgG1 | | | | | | | |
| Prolia | Anti-RANK- | SubCut. | Solution. | 60 | 5.2 | Na—Ac | 0 | Each 1 mL single-use |
| | L; Human | | | | | | | prefilled syringe of Prolia |
| | IgG2 | | | | | | | contains 60 mg |
| | | | | | | | | denosumab (60 mg/mL |
| | | | | | | | | solution), 4.7% sorbitol, |
| | | | | | | | | 17 mM acetate, 0.01% |
| | | | | | | | | polysorbate 20, NaOH. |
| Arzerra | Anti-CD20; | IV | Solution | 20 | 6.5 | Na-Cit | 5.85 | 8.55 mg/mL sodium |
| | Human IgG1 | | | | | | | citrate and 0.195 mg/mL |
| | | | | | | | | citric acid monohydrate as |
| | | | | | | | | buffering agents, 5.85 |
| | | | | | | | | mg/mL sodium chloride as |
| | | | | | | | | an isotonic agent. |
| Stelara | Anti-IL12/23; | SC | Solution | 90 | 5.7- | L-his | 0 | L-histidine and L-histidine |
| | Human IgG1 | | | | 6.3 | HCl | | monohydrochloride |
| | | | | | | | | monohydrate (1 mg/mL), |
| | | | | | | | | Polysorbate 80 (0.04 |
| | | | | | | | | mg/mL), and sucrose (76 |
| | | | | | | | | mg/mL). |
| Ilaris | Anti-IL1β; | SC | Powder | 150 | N/A | L-his | 0 | 92.38 mg/mL sucrose, and |
| | Human IgG1 | | | | | HCl | | 0.60 mg/mL polysorbate |
| | | | | | | | | 80. L-histidine and L- |
| | | | | | | | | histidine hydrochloride |
| | | | | | | | | monohydrate are used to |
| | | | | | | | | adjust and buffer pH. |
| Simponi | Anti-TNFα; | SC | Solution. | 100 | 5.5 | L-his | 0 | 0.88 mg/mL L-histidine |
| | Human IgG1 | | | | | HCl | | and L-histidine |
| | | | | | | | | monohydrochloride |
| | | | | | | | | monohydrate, 41 mg/mL |
| | | | | | | | | sorbitol, 0.16 mg/mL |
| | | | | | | | | polysorbate 80 |
| Cimzia | Anti-TNFα; | SC | Solution. | 200 | 4.7 | Na—Ac | 7.31 | 1.36 mg/mL sodium |
| | Hu-manized | | | | | | | acetate |
| | Fab pegyl. | | | | | | | |
| Soliris | Anti-C5; | IV | Solution. | 10 | 7.0 | Na | 8.77 | 0.46 mg/mL sodium |
| | Humanized | | | | | Phos. | | phosphate monobasic, |
| | IgG2/4 | | | | | | | 1.78 mg/mL sodium |
| | | | | | | | | phosphate dibasic, 0.22 |
| | | | | | | | | mg/mL polysorbate 80 |
| | | | | | | | | (vegetable origin) |
| Vectibix | Anti-EGFR; | IV | Solution. | 20 | 5.8 | Na—Ac | 5.80 | 6.8 mg/mL sodium acetate |
| | Human IgG2 | | | | | | | |
| Lucentis | Anti-VEGF; | Intra | Solution. | 10 | 5.5 | L-his | 0 | 10 mM histidine HCl, |
| | Humaniz. | ocular | | | | HCl | | 10% α,α-trehalose |
| | IgG1 Fab | | | | | | | dihydrate, 0.01% |
| | | | | | | | | polysorbate 20 |
| Tysabri | Anti-α4 | IV | Solution | 20 | 6.1 | Na | 0.8 | 1.13 mg/mL sodium |
| | integrin; | | | | | Phos. | | phosphate, monobasic, |
| | Humanized | | | | | | | monohydrate; 0.48 mg/mL |
| | IgG4 | | | | | | | sodium phosphate, |
| | | | | | | | | dibasic, heptahydrate; 0.2 |
| | | | | | | | | mg/mL polysorbate 80 |
| Avastin | Anti-VEGF; | IV | Solution. | 25 | 6.2 | Na | 0 | 60 mg/mL α,α-trehalose |
| | Humanized | | | | | Phos. | | dihydrate, 5.8 mg/mL Na |
| | IgG1 | | | | | | | phosphate (monobasic, |
| | | | | | | | | mono-hydrate), 1.2 |
| | | | | | | | | mg/mL Na phosphate |
| | | | | | | | | (dibasic, anhydrous), 0.4 |
| | | | | | | | | mg/mL polysorbate 20 |
| Erbitux | Anti-EGFR; | IV | Solution | 2 | 7.0- | Na | 8.48 | 1.88 mg/mL sodium |
| | Chimeric | | | | 7.4 | Phos. | | phosphate dibasic |
| | IgG1 | | | | | | | heptahydrate, 0.41 mg/mL |
| | | | | | | | | sodium phosphate |
| | | | | | | | | monobasic monohydrate |
| Raptiva | Anti-CD11a; | SC | Powder | 100 | 6.2 | L-his | 0 | 98.56 mg/mL sucrose, |
| | Humanized | | | | | HCl | | 5.44 mg/mL L-histidine |
| | IgG1 | | | | | | | hydrochloride |
| | | | | | | | | monohydrate, 3.44 mg/mL |
| | | | | | | | | L-histidine and 2.4 |
| | | | | | | | | mg/mL polysorbate 20 |
| Bexxar | Anti-CD20; | IV | Solution. | 14 | 7.2 | Na | 145 | 10% (w/v) maltose, 10 |
| | Murine IgG2a | | | | | Phos, | mM | mM phosphate |
| Xolair | Anti-IgE; | SC | Powder | 125 | N/A | L-his | 0 | L-histidine (1.5 mg/mL), |
| | Humanized | | | | | HCl | | L-histidine hydrochloride |
| | IgG1 | | | | | | | monohydrate (2.33 |
| | | | | | | | | mg/mL), polysorbate 20 |
| | | | | | | | | (0.42 mg/mL), sucrose |
| | | | | | | | | (121.3 mg/mL) |
| Humira | Anti-TNFα; | SC | Solution | 50 | 5.2 | Phosph | 6.16 | 0.86 mg/mL monobasic |
| | Human IgG1 | | | | | Citrate | | sNa phosphate dihydrate, |
| | | | | | | | | 1.525 mg/mL dibasic Na |
| | | | | | | | | phosphate dihydrate, 0.3 |
| | | | | | | | | mg/mL Na citrate, 1.3 |
| | | | | | | | | mg/mL citric acid |
| | | | | | | | | monohydrate, 12 mg/mL |
| | | | | | | | | mannitol, 1.0 mg/mL |
| | | | | | | | | polysorbate 80, NaOH to |
| | | | | | | | | adjust pH. |
| Zevalin | Anti-CD20; | IV | Solution | 1.6 | N/A | N/A | 9.0 | |
| | Murine IgG1- | | | | | | | |
| | tiuxetan conj. | | | | | | | |
| Campath- | Anti-CD52; | IV | Solution | 30 | 6.8- | Na—K | 8.0 | 1.44 mg/mL dibasic Na |
| 1H | Humanized | | | | 7.4 | Phos. | | phosphate, 0.2 mg/mL |
| | IgG1 | | | | | | | KCl, 0.2 mg/mL |
| | | | | | | | | monobasic K phosph-ate, |
| | | | | | | | | 0.1 mg/mL polysorbate |
| | | | | | | | | 80, 0.0187 mg/mL |
| | | | | | | | | disodium edetate |
| | | | | | | | | dihydrate [EDTA]. |
| Mylotarg | Anti-CD33; | IV | Powder | 1.0 | N/A | Na | N/A | Conj. with calicheamicin |
| | Humanized | | | | | Phos. | | N-acetyl-gamma |
| | IgG4-Conjug. | | | | | | | calicheamicin via a |
| | | | | | | | | bifunctional linker. 50% |
| | | | | | | | | of the antibody loaded |
| | | | | | | | | with 4-6 moles |
| | | | | | | | | calicheamicin per mole of |
| | | | | | | | | antibody. The remaining |
| | | | | | | | | 50% of the antibody is not |
| | | | | | | | | linked to the |
| | | | | | | | | calicheamicin derivative, |
| | | | | | | | | dextran 40; sucrose; |
| | | | | | | | | sodium chloride; |
| | | | | | | | | monobasic and dibasic |
| | | | | | | | | sodium phosphate. |
| Herceptin | Anti-HER2; | IV | Powder | 21 | ~6 | L-his | 0 | 20 mg/mL α,α-trehalose |
| | Humanized | | | | | HCl | | dihydrate, 0.495 mg/mL L- |
| | IgG1 | | | | | | | histidine HCl, 0.32 mg/mL |
| | | | | | | | | L-histidine, and 0.09 |
| | | | | | | | | mg/mL polysorbate 20, |
| | | | | | | | | 1.1% benzyl alcohol |
| Remicade | Anti-TNFα; | IV | Powder | 10 | 7.2 | Na | 0 | 50 mg/mL sucrose, 0.05 |
| | Chimeric | | | | | Phos. | | mg/mL polysorbate 80, |
| | IgG1 | | | | | | | 0.22 mg/mL monobasic |
| | | | | | | | | sodium phosphate, |
| | | | | | | | | monohydrate, and 0.61 |
| | | | | | | | | mg/mL dibasic sodium |
| | | | | | | | | phosphate dihydrate. |
| Synagis | Anti-RSV; | IM | Soltn. | 100 | 6.0 | His/Gly | 0 | 3.9 mg/mL histidine, 0.1 |
| | Humanized | | | | | HCl | | mg/mL glycine, and 0.5 |
| | IgG1 | | | | | | | mg/mL chloride. |
| Simulect | Anti-IL2R; | IV | Powder | 4 | N/A | Na—K | 0.32 | 1.44 mg/mL monobasic K |
| | Chimeric | | | | | Phos. | | phosphate, 0.2 mg/mL |
| | IgG1 | | | | | | | disodium hydrogen |
| | | | | | | | | phosphate (anhydrous, 4 |
| | | | | | | | | mg/mL sucrose, 16 mg/mL |
| | | | | | | | | mannitol, and 8 mg/mL |
| | | | | | | | | glycine |
| Zenapax | Anti-IL2R; | IV | Soltn. | 5 | 6.9 | Na | 4.6 | 3.6 mg/mL sodium |
| | Humanized | | | | | Phos. | | phosphate monobasic |
| | IgG1 | | | | | | | monohydrate, 11 mg/mL |
| | | | | | | | | sodium phosphate dibasic |
| | | | | | | | | heptahydrate, 0.2 mg/mL |
| | | | | | | | | polysorbate 80, HCl or |
| | | | | | | | | NaOH to adjust the pH. |
| Rituxan | Anti-CD20; | IV | Soltn. | 10 | 6.5 | Na-Cit | 9.0 | 7.35 mg/mL sodium |
| | Chimeric | | | | | | | citrate dihydrate, 0.7 |
| | IgG1 | | | | | | | mg/mL polysorbate 80 |
| Reopro | Anti- | IV | Soltn. | 2 | 7.2 | Na | 150 | 0.01M sodium phosphate, |
| | GPIIb/IIIa; | | | | | Phos. | mM | 0.001% polysorbate 80 |
| | Chimeric | | | | | | | |
| | IgG1 Fab | | | | | | | |

Na—Ac: Sodium acetate
Na-Cit: Sodium citrate;
Na-Phos. Sodium phosphate
For NaCl, 150 mM is ~9 mg/mL

In some embodiments, antibodies of this invention can be is supplied at a concentration of 10 mg/mL in either 100 mg (10 mL) or 500 mg (50 mL) single-use vials. The product can be formulated for IV administration in 9.0 mg/mL sodium chloride, 7.35 mg/mL sodium citrate dihydrate, 0.7 mg/mL polysorbate 80, and Sterile Water for Injection. The pH can be adjusted to 6.5.

In other embodiments, antibodies can be in the form of a preservative-free lyophilized powder for intravenous (IV) administration in a vial. The content of each vial can be 440 mg antibody, 400 mg α-α-trehalose dihydrate, 9.9 mg L-histidine HCl, 6.4 mg L-histidine, and 1.8 mg polysorbate 20. Reconstitution can be performed using 20 mL Bacteriostatic Water for Injection (BWFI), USP, and can contain 1.1% benzyl alcohol as a preservative. This yields a multi-dose solution containing 21 mg/mL antibody, at a pH of approximately 6.

In still further embodiments, antibodies can be prepared in a sterile, pH 6.2 solution for intravenous infusion. Antibodies can be supplied in 100 mg and 400 mg preservative-free, single-use vials to deliver 4 mL or 16 mL of the antibody (25 mg/mL). A 100 mg product can be formulated in 240 mg α,α-trehalose dihydrate, 23.2 mg sodium phosphate (monobasic, monohydrate), 4.8 mg sodium phosphate (dibasic, anhydrous), 1.6 mg polysorbate 20, and Water for Injection, USP. A 400 mg product can be formulated in 960 mg α,α-trehalose dihydrate, 92.8 mg, sodium phosphate (monobasic, monohydrate), 19.2 mg sodium phosphate (dibasic, anhydrous), 6.4 mg polysorbate 20, and Water for Injection, USP.

In alternative embodiments, human IgG1κ monoclonal antibodies can be produced in a well-characterized recombinant cell line and is purified using standard bio-processing technology. The manufacturing process contains steps for the clearance of viruses. IgG1 antibodies can be available as 45 mg of antibody in 0.5 mL and 90 mg of antibody in 1 mL, supplied as a sterile solution in a single-use prefilled syringe with a 27 gauge fixed ½ inch needle, or a single-use 2 mL Type I glass vial with a coated stopper. The syringe can be fitted with a passive needle guard and a needle cover that is manufactured using a dry natural rubber (a derivative of latex). For the 45 mg antibody preparation, a prefilled syringe also contains L-histidine and L-histidine monohydrochloride monohydrate (0.5 mg), Polysorbate 80 (0.02 mg), and sucrose (38 mg) for a final volume of 0.5 mL An alternative formulation can contain 90 mg antibody in prefilled syringe containing: L-histidine and L-histidine monohydrochloride monohydrate (1 mg), Polysorbate 80 (0.04 mg), and sucrose (76 mg) to fill to a final volume of 1 mL. A 45 mg antibody preparation in a vial can contain: L-histidine and L-histidine monohydrochloride monohydrate (0.5 mg), Polysorbate 80 (0.02 mg), and sucrose (38 mg) at a final volume of 0.5 mL. Solutions can be at a pH of 5.7-6.3.

Additionally, antibodies of this invention can be used in diagnostic kits, which can contain antibodies, antibodies linked to streptavidin or biotin (for conjugation), solubilizing agents, mixing vials, and instructions for carrying out in vitro analysis of the presence of CCR7 in samples obtained from human beings or other animals that express CCR7. For example, biotinilation of anti-CCR7 antibodies using a commercial biotinilation reagent EZ-Link Sulfo-NHS-LC-Biotin™ (ThermoScientific, Catalog Number 21335) was performed as per the manufacturer recommendation and so derivatized antibodies displayed binding EC₅₀ comparable to that for original antibodies. The biotinilated antibodies bound to CCR7 on PBMCs and CCR7-expressing reporter cell lines were further stained with Streptavidin-PE and cells were analyzed with fluorescence flow cytometer. Antibodies can also be linked to detectable tags (e.g., fluorescent tags) enabling their detection using a variety of analytic methods.

As can be appreciated from the above descriptions, there are animal models that can be used to develop treatments for disorders in humans characterized by CCR7-ligand interactions, and these animal systems are reasonably predictive of their effects in diagnosis, prognosis, and treatment of human disease.

### EXAMPLES

The following examples are presented to illustrate aspects and embodiments of this invention. As such, they are not intended to limit the scope of the invention. Rather, persons of skill in the art can use the descriptions and teachings herein to create, modify or produce other human antibodies and uses thereof without undue experimentation. All such embodiments are considered part of this invention.

### Example 1

### Binding of Anti-CCR7 Antibodies to Cell Lines

To determine whether fully human antibodies of this invention bind to CCR7 or other GPCRs, we carried out studies using a series of cell lines in culture.

Cell Lines Expressing Human GPCRs

The cell lines used were:
1. CHO-K1 (Chinese Hamster Ovary cells, ATCC Cat #CCL-61);
2. BHK-21 (Syrian Hamster Fibroblasts, ATCC Cat #CCL-10);
3. CF2Th (Canine Thymocytes, ATCC Cat #CRL-1430);
4. R1610 (Chinese Hamster Lung Fibroblasts, ATCC Cat #CRL-1657); and
5. HEK-293T (Human Embryonic Kidney cells, Cat #CRC-1573).

The same cell lines were also adapted to stable express six different G-Protein Coupled Receptors (GPCRs). Next GPCRs have been expressed and used in the work: human CCR7, human CCR5, Human CXCR2 (hCXCR2), human CXCR3, human FPR, and mouse CCR7.

The mammalian cells adapted to stable expression of GPCRs included:
1. CHO-K1-hCCR7 (Chinese Hamster Ovary cells CHO-K1 expressing human chemokine receptor CCR7);
2. CHO-K1-hFPR (Chinese Hamster Ovary cells CHO-K1 expressing human Formyl Peptide Receptor FPR-1);
3. CHO-K1-hCCR5 (Chinese Hamster Ovary cells CHO-K1 expressing human CCR5);
4. BHK-21-hCCR7 (Syrian Hamster Fibroblasts BHK-21 expressing human CCR7);
5. CF2Th-hCXCR2 (Canine Thymocytes expressing human CXCR2);
6. CF2Th-hCXCR3 (Canine Thymocytes expressing human CXCR3);
7. R1610-hCCR7 (Chinese Hamster Lung Fibroblasts expressing human CCR7);
8. HEK-293T-hFRR-1 (Human Embryonic Kidney cells expressing human FPR-1); and
9. CHO-K1-hCCR7 (Chinese Hamster Ovary cells CHO-K1 expressing mouse chemokine receptor CCR7).

In addition, other cell lines expressing human and orthologous GPCRs that also belong to the subclass of chemokine receptors and are closely related to CCR7 were generated and tested for the expression of respective GPCRs using staining with commercially available anti-respective-GPCR conjugates with fluorescent moiety PE:

R1610-human CXCR1 (Extracellular staining with anti-human CXCR1 mouse antibody conjugated to PE. BD Pharmigen, Cat. #555940).

Cf2Th-human CXCR2 (Extracellular staining with anti-human CXCR2 mouse antibody conjugated to PE. BD Pharmigen, Cat. #555933).

R1610-human CXCR3 (Extracellular staining with anti-human CXCR3 mouse antibody conjugated to PE. R&D Systems, Cat. #FAB160P).

Cf2Th-human CXCR4 (Extracellular staining with anti-human CXCR4 CD184/12G5 mouse antibody conjugated to PE. BD Pharmigen, Cat. #557145).

CHO-human CXCR5 (Extracellular staining with anti-human CXCR5 mouse antibody conjugated to PE. R&D Systems, Cat. #FAB 190P).

CHO-human CXCR6 (Extracellular staining with anti-human CXCR6 mouse antibody conjugated to PE. R&D Systems, Cat. #FAB699P).

CHO-human CXCR7 (Extracellular staining with anti-human CXCR mouse antibody conjugated to PE. R&D Systems, Cat. #FAB42271P).

CHO-human CCR3 (Extracellular staining with anti-human CCR3 mouse antibody conjugated to PE. BD Pharmigen, Cat. #558165).

CHO-human CCR4 (Extracellular staining with anti-human CCR6 mouse antibody conjugated to PE. R&D Systems, Cat. #FAB1567P).

CHO-human CCR5 (Extracellular staining with anti-human CCR5 mouse antibody conjugated to PE. BD Pharmigen, Cat. #556042).

CHO-human CCR6 (Extracellular staining with anti-human CCR6 mouse antibody conjugated to PE. R&D Systems, Cat. #FAB 195P)

CHO-cyno CCR6 (Extracellular staining with anti-human CCR6 mouse antibody conjugated to PE. R&D Systems, Cat. #FAB195P)

CHO-mouse CCR6 (Intracellular staining with Streptavidin PE R&D Systems, Cat. #F0040) CHO-human CCR7 (Extracellular staining with anti-human CCR7 mouse antibody conjugated to PE. BD Pharmigen, Cat. #12-1979-42)

R1610-human CCR7 (Extracellular staining with anti-human CCR7 mouse antibody conjugated to PE. BD Pharmigen, Cat. #12-1979-42)

CHO-mouse CCR7 (Extracellular staining with anti-human CCR7 mouse antibody conjugated to PE. BD Pharmigen, Cat. #12-1979-42)

R1610-human CCR9 (Extracellular staining with anti-human CCR9 mouse antibody conjugated to PE. BD Pharmigen, Cat. #557975).

CHO-human CCR10 (Extracellular staining with anti-human CCR10 mouse antibody conjugated to PE. R&D Systems, Cat. #FAB3478P)

CHO-cyno CXCR3 (Extracellular staining with anti-human CXCR3 mouse antibody conjugated to PE. R&D Systems, Cat. #FAB 160P).

Detection of Binding of Antibodies to Human GPCRs Expressed in Cells Cell staining procedure was as follows: 5,000-10,000 cell suspension in 10 μl FACS buffer (1×PBS, 2.0% FBS, 0.2% sodium azide) was mixed with 10 μl of 200 nM the corresponding anti-CCR7 MAB and incubated on ice for 30 min. Washing step—after the incubation 150 μl of FACS buffer was added to the cell sample, the samples were mixed gently by up-down pipetting the cell suspension. Then the samples were centrifuged at 1100 rpm for 5 minutes, and supernatants were removed. Washing step was repeated ones. Then 10 μl of anti-human PE-(Fab)2 form Jackson Immuno Research Lab. #709-116-098 diluted 40 times in FACS buffer were added to the cells and the cells were re-suspended by up-down pipetting. After 20 min. incubation on ice in dark the washing step was repeated twice. The washed cells were mixed with 100 μl of FIX buffer (0.5% Paraformaldehyde solution in PBS). Fixed samples were stored in dark on ice and analyzed by FACS on Guava PCA-96 flow cytometer.

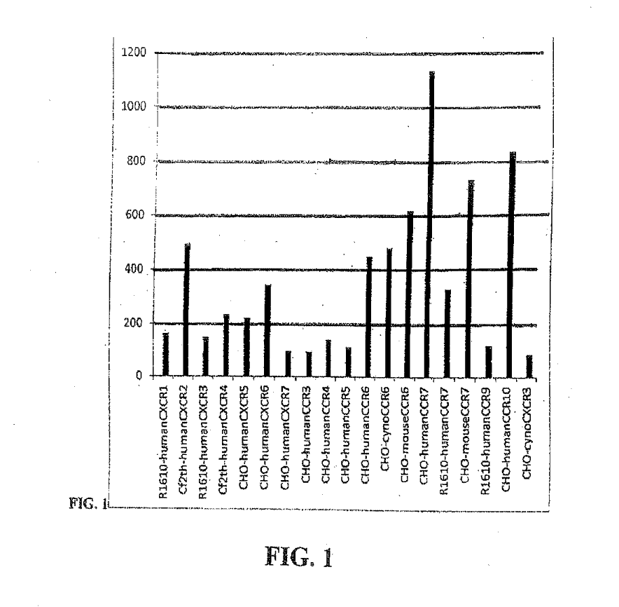

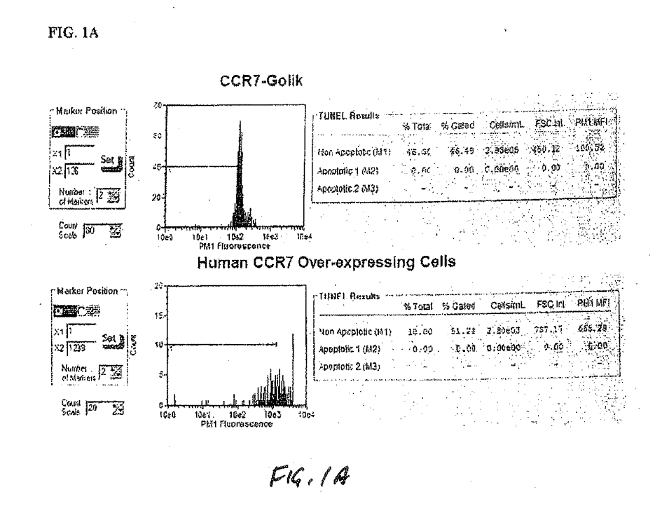

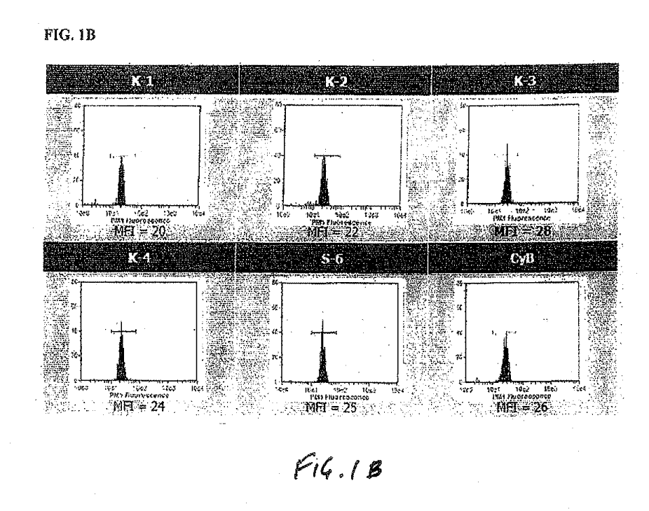

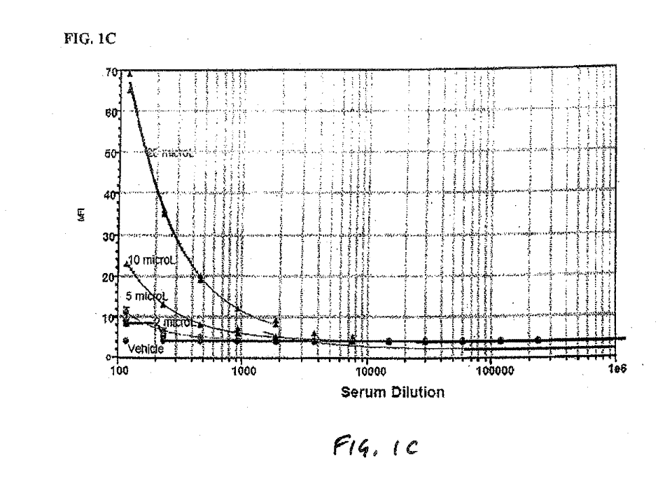

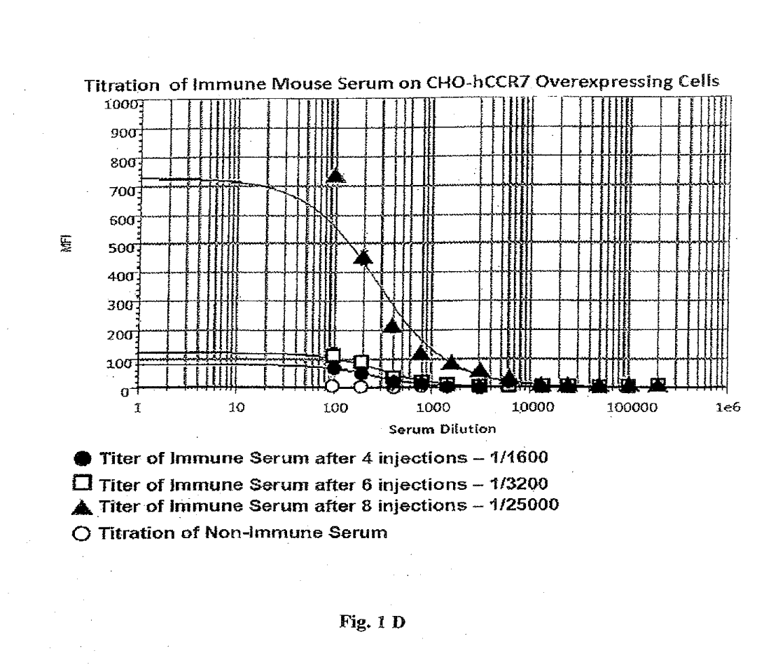

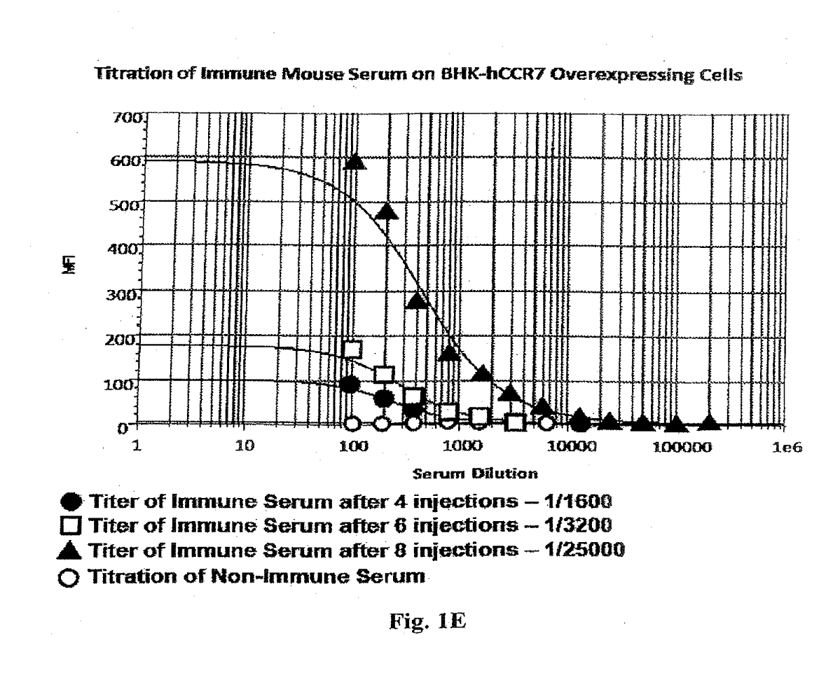

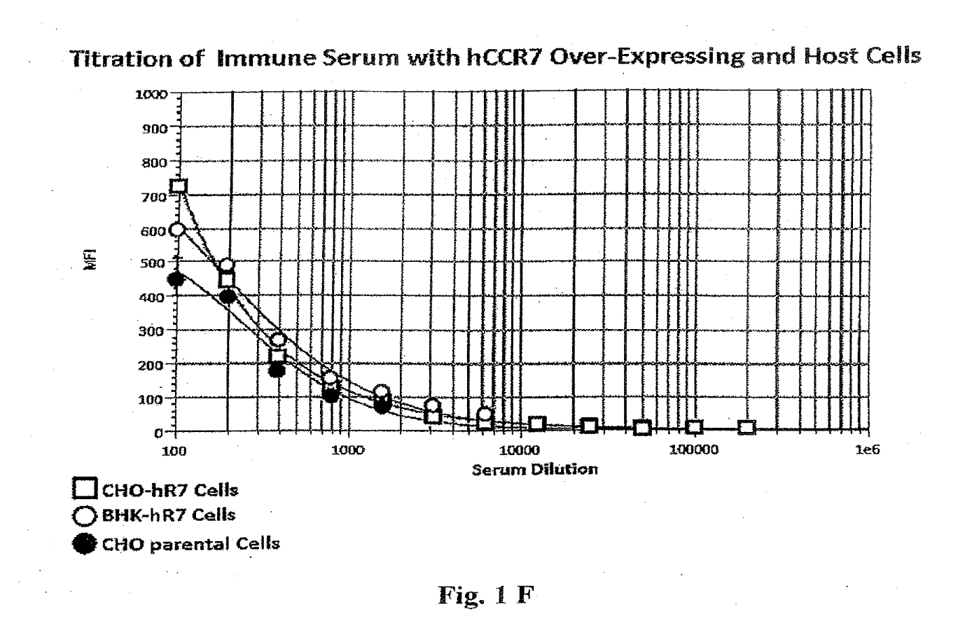

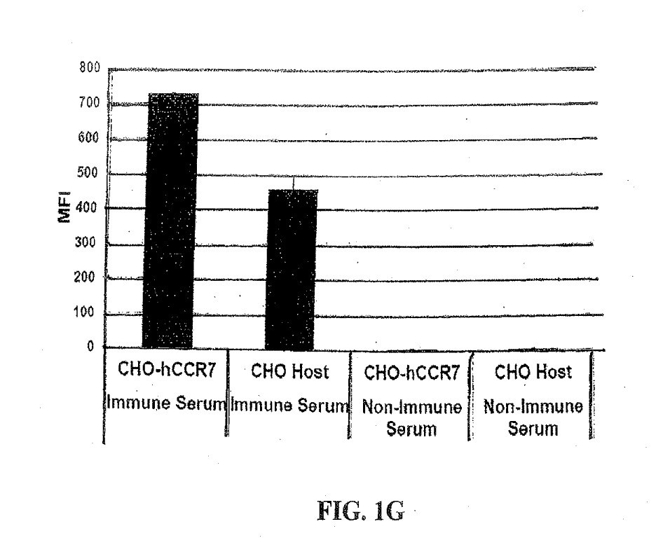

The data so obtained are provided in FIG. 1. The staining confirmed that all cell lines so produced display a high level of expression of their respective GPCR and thus were suitable for analysis of specificity of binding of anti-CCR7 antibodies of this invention.

### Example 2

### Construction and Expression of Codon-Optimized CCR7 (synCCR7)

For certain embodiments, methods for construction and expression of codon-optimized CCR7 are described, in general, in Mirzabekov et al., Enhanced Expression, Native Purification, and Characterization of CCR5, a Principal HIV-1 Coreceptor. J. Biol. Chem. 274(40)29745-28750 (1999), expressly incorporated herein fully by reference.

In certain aspects, analysis of codon usage for 45 GPCRs representing different protein subfamilies was performed with Genbank™ data and software developed by the University of Wisconsin Genome Sequence Group. The sequence encoding human CCR7 was optimized for mammalian cell codon usage, utilizing the following codons: alanine (GCC), arginine (CGC), asparagine (AAC), aspartic acid (GAC), cysteine (TGC), glutamic acid (GAG), glutamine (CAG), glycine (GGC), histidine (CAC), isoleucine (ATC), leucine (CTG), lysine (AAG), methionine (ATG), phenylalanine (TTC), proline (CCC), serine (ICC), threonine (ACC), tryptophan (TGG), tyrosine (TAC) and valiine (GTG). The 5′ and 3′ sequences flanking the CCR7 coding sequence were modified. Following restriction sites for EcoRV, EcoRI and HindIII, the Kozak consensus (GCCGCCACCATGG; SEQ ID NO:1) was placed immediately 5′ to the CCR7 reading frame. A sequence encoding a single glycine residue followed by the bovine rhodopsin C9 peptide tag (TETSQVAPA; SEQ ID NO:2) was introduced immediately 5′ to the natural stop codon of CCR7. At the 3′ end of the epitope-tagged CCR7 gene. XhoI, SalI, and NotI restriction sites were introduced. Analogous constructs were made for the wild-type human CCR7 gene and the bovine rhodopsin gene, except that the codons were not altered and, in the latter case, the C-terminal C9 sequence was naturally present.

Oligonucleotides, each approximately 70 nucleotides in length, corresponding to the complete sense and antisense strands of the synCCR7 gene and flanking sequences, were constructed so that approximately 50% of their sequences were complementary to those of each of the two complementary oligonucleotides from the opposite strand. Oligonucleotides were deprotected in pure ammonium hydroxide at 65° C. for 4 h, after which the ammonium hydroxide was evaporated, and the oligonucleotides were dissolved in water at a final concentration of 2 nM. For gene synthesis, the oligonucleotides were separated into groups (about 6 to 8 oligonucleotides per group) and about 25 cycles of polymerase chain reaction (PCR) were performed using Pfu polymerase (Stratagene, La Jolla, Calif.) and a 3-fold molar excess of the 5′ and 3′ terminal oligonucleotides in each group. This step generated small segments of the sysCCR7 gene with complementary and overlapping ends. Equal amounts of each PCR product were combined with a 3-fold molar excess of the 5′ and 3′ terminal oligonucleotides of the complete synCCR7 sequence. A second round of about 25 cycles of PCR yielded the complete sysCCR7 sequence. The product was sequenced to ensure that the sequence was correct.

The synCCR7, wild-type CCR7 and bovine rhodopsin sequences were cloned into the following vectors: PMT4 (a gift from Dr. Reeves, Massachusetts Institute of Technology), PACH (a gift from Dr. Velan, Israel Institute for Biological Research), pcDNA 3.1(+) and pcDNA4/HisMax (Invitrogen), and PND (a gift from Dr. Rhodes, University of California, Davis). After cloning the synCCR7 gene into the pcDNA4/HisMax vector, the sequence encoding the N-terminal HisMax region was removed by QuikChange™ mutagenesis (Stratagene). Different cell lines were transfected with the synCCR7 gene and wild-type CCR7 genes using the GenePorter™ transfection reagent (San Diego, Calif.). Following transfection, cells expressing CCR7 were selected with 0.8 mg/ml of neomycin (G418). Cells expressing the highest surface levels of CCR7 were selected by fluorescence activated cell sorting (FAGS) after staining cells with R-phycoerythrin-conjugated anti-CCR7 antibody (Pharmagen, San Diego, Calif.). The highest synCCR7 expressing cells were selected by FACS.

Alternatively, a number of service providers, such as for example Genewiz (http_://_www_.genewiz_.com/_public/gene-_synthesis_.aspx) currently offer commercial service of synthesis of protein genes, and the CCR7 genes synthesis do not represent a problem for such companies.

### Example 3

### Radiolabeling and Immunoprecipitation of CCR7

Approximately 4×10⁸ CCR7-expressing Cf2Th or HEK-293T cells grown to full confluence in 100-mm dishes were washed twice in PBS and starved for 1 h at 37° C. in Dulbecco's modified Eagle's medium without cysteine and methionine (Sigma) or in sulfate-free media (ICN, Costa Mesa, Calif.). The starvation medium was removed and 200 μCi each of [³⁵S]methionine and [³⁵S]cysteine or 500 μCi of [³⁵S]sulfate (NEN Life Science Products) in 4 ml of medium was added to the cells for various times for pulse-chase experiments or overnight (12 h) in other cases. Cells were washed twice with PBS and lysed in 1 ml of solubilization medium composed of 100 mM (NH₄)₂ SO₄, 20 mM Tris-HCl (pH 7.5), 10% glycerol, 1%(w/v) detergent (see below), and Protease Inhibitor Mixture (one tablet of Complete™ (Roche Molecular Biochemicals) per 25 ml.

The lysate was incubated at 4° C. for 30 minutes on a rocking platform, and cell debris was removed by centrifugation at 14,000×g for 30 min. CCR7 was precipitated with 20 μl of 1D4-Sepharose beads overnight, after which the beads were washed six times in the solubilization medium and pelleted. An equal volume of 2×SDS-sample buffer was added to the beads, followed by re-suspension and incubation for 1 h at 55° C. Samples were run on 11% SDS-polyacrylamide minigels and visualized.

### Example 4

### Solubilization Buffers

Detergents were used as components of solubilization buffers. The detergents, with abbreviations and critical micelle concentrations in parentheses, were n-octyl-β-D-glucopyranoside (23.4 mM), n-decyl-β-D-maltoside (1.8 mM), n-dodecyl-β-D-maltoside (DDM; 0.17 mM), cyclohexyl-butyl-β-D-maltoside (Cymal™-4; 7.6 mM), cyclohexyl-pentyl-β-D-maltoside (Cymal™-6; 0.56 mM), cyclohexyl-heptyl-β-D-maltoside (Cymal™-7; 0.19 mM), cyclo-hexylpropanoyl-N-hydroxyethylglucamide (108 mM), cyclohexylbutanoyl-N-hydroxyethylglucamide (35 mM), cyclohexylpentanoyl-N-hydroxyethylglueamide (11.5 mM), N-oetylphosphocholine (Fos-Choline™ 8; 114 mM), N-decylphosphocholine (Fox-Choline™ 10; 1 mM), N-dodecylphosphocholine (Fos-Choline™ 12; 1.5 mM), N-tetradecylphosphocholine (Fos-Choline™ 14; 0.12 mM), Triton X-100 (0.02 mM), CHAPS (8 mM), Nonidet P-40 (0.02 mM), and diheptanoyl-phosphocholine (DHPC; 1.4 mM). All detergents were purchased from Anairace (Maumee, Ohio) except DHPC, which was purchased from Avanti Polar Lipids (Alabaster, Ala.).

### Example 5

### Purification of CCR7

Stable Cf2Th/PACH/synCCR7 cells grown to full confluence in a 150 mm dish were incubated with medium containing 4 mM sodium butyrate for 40 h, washed in PBS, detached by treatment with 5 mM EDTA/PBS, pelleted, and again washed in PBS. Cells were solubilized for 30 min with 3 ml of the solubilization medium containing Cymal™-5 and centrifuged for 30 min at 14,000×g. The cell lysate as incubated with 50 μl of 1D4-Sepharose beads on a rocking platform at 4° C. for 10-12 h. The Sepharose™ beads were washed about five times with the washing buffer (100 mM (NH₄)₂SO₄, 20 mM

Tris-HCl (pH 7.5), 10% glycerol and 1% Cymal™-5) and once with washing buffer plus 500 mM MgCl₂. CCR7 was eluted from the beads by three successive washes with 50 μl of medium containing 200 mM C9 peptide (TETSQVAPA: SEQ ID NO: 2), 500 mM MgCl₂, 100 mM (NH₄)₂SO₄, 20 mM Tris-HCl (pH 7.5), 10% glycerol, and 0.5% Cymal™-5. The amount of CCR7 was estimated by Coomassie Blue staining of an SDS-polyacrylamide gel (SDS-PAGE) run with standard quantities of bovine serum albumin.

In other embodiments, CCR7 can be obtained using paramagnetic particles, chemically derivatized with a capture agent, using the protocol provided by the Dynal Biotech Inc. A capture reagent can be an antibody capable of selective binding a tag or streptavidin that can bind a known peptide tag; either of the tags can be attached at the C-terminus of CCR7.

CCR7 protein can be over-expressed in a mammalian cell by transfecting, using for example, a GenePORTER™ transmembrane reagent and protocol (Gelantis), a line of mammalian cells (which can be purchased from ATCC) with a vector (for example, pcDNA3.1, from Invitrogen) carrying the gene of the protein having an appropriate peptide tag at the C-terminus and genes that provide an antibiotic resistance to the cells. In some embodiments, CCR7 monomers can each have a C-terminal tag, and in other embodiments, some CCR7 monomers can have C-terminal tags and other CCR7 monomers can be untagged. For manufacture of hetero-multimeric proteins of this invention, cells can be transfected with vectors that encode tagged monomers and other vectors that encode un-tagged monomers. Alternatively, a single vector having two or more expression cassettes, one cassette having a sequence encoding a tagged monomer and another cassette encoding an untagged monomer) can be used. In such systems, a mixture of tagged and untagged monomers can be produced, that when associated with each other in a cell, can form a hetero-multimeric protein complex. Antibiotic resistance (for example, resistance to gentamycin (Geneticin™; G418), the feature acquired concomitantly with the capacity to over-express CCR7, can be used for selecting over-expressing cells that survive in the presence of added antibiotic.

Cells that over-express CCR7 can be harvested, and the membranes of the cells can be solubilized in a mixture of detergents. Solublilized CCR7 (and other solubilized proteins) can be clarified by centrifugation and the CCR7-containing protein-detergent complexes can be mixed with beads carrying a capture reagent capable of binding to the tag on the CCR7 protein.

Washing the beads can remove contaminants from the CCR7-detergent complexes. A magnet can be used to hold beads within a vessel (e.g., tube) and washing solutions can be added to carry away non-bound materials, including contaminants. Beads retaining CCR7 in the desirable orientation (i.e., the extracellular portion is exposed on the surface of the bead) can then be dialyzed to produce complexes having the desired detergents.

### Example 6

### Preparation of CCR7 Target Presentation Materials, CCR7-Golik™ and CCR7-FMPL, of this Invention

Search for antibodies or antibody fragments that bind external domains of membrane proteins remains to be extremely laborious and inefficient process. Two major approaches to the discovery of such ligands employ:

(1) Immunization with preparations of trans-membrane protein, such as cells, viral particles, or cellular membranes, or with peptide fragments of a trans-membrane protein, and discovery of antibodies that bind the target from obtained by immunization plurality of cells expressing various antibodies via formation of hybridoma cells or focused libraries obtained by PCR of B-cells from the serum of immunized animals; or

(2) A phage or other type of library containing a very large number of antibodies or antibody fragments linked to their respective genotype information (phage libraries, molecular libraries, mammalian cells libraries, bacterial libraries, yeast libraries, in which a member carries an antibody portion capable of binding to an antigen and genetic information on the variable portions of such antibody). Screening of such a library can result in the isolation from the library containing those library members that bind to a preparation of trans-membrane protein, such as cells, viral particles or cellular membranes, or to peptide fragments of a trans-membrane protein.

Both approaches are dependent upon the quality of membrane protein preparation, in the following referred to as Target Presentation Material (TPM).

(a) When a trans-membrane-protein in a TPM is present at a low concentration, immune response can be poor, and isolation of antibodies that bind to the protein can be problematic.

(b) The presence of protein and non-protein contaminants in the TPM, which is typically the case when whole cells, virus particles, or crude membrane preparations are used, can produce a background signal sufficiently high to make identification the ligands in question difficult.

(c) The loss of native conformation of such a protein can render both approaches inefficient and difficult. The important requirements for quality (a) through (c) are poorly addressed by prior art cell-based, viral particle-based, liposome-based, or membrane-based preparations of membrane proteins. Also, the use of prior art fragments of membrane proteins has been proven inefficient because such fragments may not maintain native conformation, and even when a ligand that binds to such a peptide is discovered, such a ligand often is unable to exert a desirable change upon the function of a membrane protein.

Magnetic Proteoliposomes (MPLs) disclosed in the U.S. Pat. No. 6,761,902 titled ‘Proteoliposomes containing an integral membrane protein having one or more transmembrane domains’ by Joseph Sodroski and Tajib Mirzabekov, Jul. 13, 2004, and the US patent application 20010034432, A1, Oct. 25, 2001 titled ‘Proteoliposomes containing an integral membrane protein having one or more transmembrane domains’ by the same inventors, and the US patent application 20040109887, A1, Jun. 10, 2004 titled ‘Immunogenic proteoliposomes, and uses thereof’ by Wyatt, Richard T. et al incorporated herein fully by reference, have been used as membrane protein preparations. MPLs allow one to purify a membrane protein in its native, functional conformation, and stabilize the protein in proper orientation and at high concentration on the surface of easy-to-handle magnetic beads. Membrane proteins in MPLs remain functionally intact due to carefully crafted membrane environment that encompasses certain added lipids. While MPLs have been proven effective in human antibody development using both transgenic mouse immunization and by selection of antibodies from phage display libraries, the need for carefully crafted and laborious selection of lipids and lipid reconstitution procedures makes this approach time consuming, expensive, and demanding highly sophisticated labor.

In one embodiment of present invention, methods of manufacturing and use of membrane-protein carrying particles that do not require laborious and expensive lipid-involving procedures are disclosed. The particles of this invention can carry membrane protein molecules on their surface that are in proper orientation, highly concentrated and can be stabilized by certain detergents in native-like or native state (“naked particles”, or “Golik™ particles”). Using such naked particles can dramatically reduce the time for selection of ligands from various libraries, such as chemical library, phage, aptamer, shpigelmer, nanobody, antibody fragment, scFv, minibody, anticalin or other protein scaffold library, cell library, and any other library.

One of the embodiments of present invention discloses naked particles that carry the CCR7 protein on their surface, and yet another embodiment of present invention discloses antibody-ligands that can bind the CCR7 protein exposed on the surface of these preparations and CCR7 on the surface of cells.

In one embodiment, manufacture of CCR7-naked particles was accomplished of the following protocol: First, paramagnetic particles, for example M-280 Tosylactivated Dynabeads™ produced by Dynal Biotech Inc. were chemically derivatized with a capture agent, using protocol provided by the Dynal Biotech Inc. A capture agent was an antibody that is capable of selective binding a respective tag, or streptavidin that can bind a known peptide tag (also, Streptavidin-coupled Dynabeads already having their surface derivatized with streptavidin are commercially available can be used); either of the tags was genetically attached at the C-terminus of a given membrane protein.

Second, a given membrane protein (in this case, human CCR7 or its ortholog, having a Strep-tag capable of binding to its respective capture agent, Streptavidin on the bead surface) was over-expressed in a mammalian cell culture.

Third, cells that over-expressed the CCR7 protein were harvested, and the membranes of the cells were solubilized in a detergent, or mixture of detergents, or in mixture of detergents also containing lipids (e.g. phosphatidyicholine, phosphatidylserine, phosphatidethanolamine, or lipid mixtures isolated form tissues or plants, or cholesterol hemisuccinate (CHS). Solubilization was performed by suspending pelleted cells in 3-4 volumes of the SB buffer and incubating 30 min on ice.

Fourth, the clarified by centrifugation (3,000 rpm, 10 min in Eppendorf CF 5417R, pellet discarded) solubilization solution containing CCR7 along with numerous other contaminating proteins was mixed with 1-2 volumes of FACS buffer and added the beads (pre-wash beads in FACS buffer, then add 50 μL beads per 10,000,000 over-expressing cells each containing ˜10⁵-10⁶ CCR molecules from which CCR7 was solubilized). The solution with the beads was incubated overnight under slow rotation.

Fifth, upon washing off contaminants by twice washing with FACS buffer (each time with 1 mL FACS buffer) that was performed retaining beads via a magnet, paramagnetic beads retaining via the tag-capture agent non-chemical bond the CCR7 protein in the desirable orientation (i.e., the extracellular portion is exposed on the surface of the bead) were produced. The CCR7-naked particles were then confirmed to retain binding of commercial anti-CCR7 antibody-PE conjugate by fluorescence flow cytometry, as depicted in FIG. 1A, which depicts original fluorescence flow cytometry data obtained using Guava PCA-96 instrument for human CCR7 expressing CHO cells (the lower panel) and for the CCR7 Target Presentation Material in the form of naked particles of this invention (the upper panel).

In one series of embodiments, CCR-7-naked particles were prepared using various SB buffer compositions, and preparations of CCR7-naked particles with the highest MFI observed using commercial CCR7 antibodies were chosen for further selections from phage libraries or for animal immunization. Selections were performed in FACS buffer or SB diluted by FACS buffer 5 times. Immunization was performed using CCR7-naked particles transferred to PBS.

In contrast to the MPL preparation, no step of addition of lipid in order to reconstitute the lipid bilayer was employed. Presumably the lipid bilayer was not reconstituted completely, and only molecules of detergent-lipid mixture stabilize the protein structure.

Production of CCR7 Naked Particles

In one series of embodiments, CCR7-naked particle preparations were produced employing the following Solubilization Buffer (SB buffer) compositions: All compositions contained 20 mM Tris-HCl, pH 7.5 and 100 mM (NH₄)SO₄, and detergents or detergent/lipids: either 1% CHAPSO (composition K-1), or 1% CHAPS plus 0.1% CHS (K-2), or 1% DDM and 0.1% CHS (K-3), or 0.5% DDM plus 0.5% CHAPS plus 0.1% CHS and 10% Glycerol (K-4), or 1% DDM (S-6), or 1% Cymal-5 (CyB). The highest MFI signal for the SB buffer containing 1% DDM and 0.1% CHS (K-3) was observed by Guava PCA-96 measurements and this SB composition was used in further selections and immunizations. For each selection or immunization a freshly prepared CCR7-naked particle TPM was used, upon quality control—MPI exceeding non-stained beads by at least 20 times.

Another embodiment of this invention includes CCR7-FMPL and its use for FACS selections. Similarly, the SB buffer composition for this IMP was optimized as described above for CCR7-naked particles and depicted in FIG. 1B, which provides original fluorescence flow cytometry data obtained using Guava PCA-96 instrument for the CCR7 Target Presentation Material prepared at various Solubilization Buffer compositions in the form of FMPLs of this invention. CCR7-FMPL preparation of this invention differs from the above disclosed CCR7-naked particle preparation in one respect—the beads, in addition to carrying a membrane protein on the surface as CCR7-naked particle preparation can emit light (fluorescence, bioluminescence, chemiluminescence, phosphorescence, etc.) and paramagnetic-, or plain fluorescence-tagged beads can be employed. Such beads are commercially available; for example, the beads (Sherotech's product FSVM-02556-2), which are Streptavidin Coated Fluorescent Magnetic Particles with 0.2-0.39 μm diameter and Nile Red staining. Nile Red excites at 485 nm, and emits at 525 nm, so it quite likes the GFP fluorescence (excites (I) at 395 nm and (II) at 475-498 nm, emits at 509 nm) and FITC (excites at 493 nm and emits at 525 nm) can be used for selection using capabilities of Fluorescence Activated Cell Sorting (FACS). Binding of CCR7-FMPL to cells expressing anti-CCR7 antibodies or antibody fragments, such as B-cells from the spleen or bone marrow of animals (mice, rats, rabbits, camelides, etc.) immunized with CCR7-TMP, hybridoma cells, yeast cells, or bacterial cells from a library is performed first by incubating of the cells with CCR7-FMPL, and then those cells that have an antibody binder on the surface capable by binding CCR7 are separated from others using the fluorescence of the beads as a criteria. Thus, FACS-based selection of anti-CCR7 antibodies can be performed.

### Example 7

### Immunization

In other aspects, this invention includes immunization of mice having fully human immune systems. Such mice are known in the art and need not be described further herein. Mice are immunized with CCR7 protein and splenocytes isolated. The genetic components of splenocytes can be placed in phage display libraries constructed from splenocytes, and analyzed using phage display technology. Based on these methods, selection of clones that express antibodies against CCR7 can be obtained (see below). By immunizing such mice with isolated, purified synCCR7, antibodies can be produced against the CCR7 protein in its native configuration. In certain of these embodiments, antibodies of this invention can recognize the ectodomain of the CCR7, and thus, can bind to native CCR7 expressed in cells, including human cells. Thus, such anti-CCR7 antibodies can be used therapeutically or diagnostically, as explained further herein.

Alternatively, wild type mice, rats, rabbits, llamas, or other animals can be immunized with CCR7-TPM or this invention. Anti-CCR7 antibody expressing cells can be then obtained from the pool of B-cells or the B-cell-obtained hybridoma cells, or from a library generated by means of PCR of the pool of cells followed by incorporating the antibody fragments into phage display or other library and isolating anti-CCR7 binders by the CCR-TMP of this invention. Then the antibodies can be humanized as known to those skillful in the art so their amino acid composition of all other than CDRs of heavy and light chains can be made over 85% identical to that of fully human antibody, and in certain embodiments over 90%, and in more desirable over 95% identical. The humanization can be performed for anti-CCR7 antibodies derived from immunization of wild type mice using TMP combination, namely CCR7-Golik and CCR7-overexpressing cells, of this invention employing the immunization protocol of this invention. While the provided protocol provided a robust immune response, other embodiments that are the modifications of the protocol employing other TMP combinations with the cells, or only CCR7-Golik, or only CCR7-FMPL can also be used to obtain a robust immune response.

One embodiment of this invention discloses immunization procedure in which CCR7-Golik was i. p. injected into wild type mice on day 1, and 9, followed by CHO-human CCR7 overexpressing cells (2×10⁶ cells/mouse) on day 16, then again CCR7-naked particle preparations on day 29, blood collection on day 32 (Titer was 1/1,600), then CCR7-naked particle preparations on day 37 and day 55, then blood collection on day 58 (Titer was 1/3,200), then on day 61 BHK-human CCR7 overexpressing cells (2×10⁶ cells/mouse), then again CCR7-naked particle on day 75, and blood collection and animal sacrifice on day 78 (Titer was 1/25,000). Groups comprised of 2-4 mice were employed, in which 2 μL, 54, 10 or 20 μL CCR7-naked particle preparation were i. p. injected (the number of beads per injection were roughly equivalent that of in the commercial Streptavidin Dynabeads™ preparation used to prepare the CCR7-TPM as described above. In control group, PBS was used as vehicle for injection.

FIG. 1C depicts a graph of fluorescence of CHO cells expressing human CCR7 at varying concentration of serum from mice immunized with 2, 5, 10, or 20 μL of CCR7-naked particle preparation of this invention according the immunization protocol of this invention the immune response in all mice groups was dependent upon the dose of the CCR7-naked particles used as immunogen, as compared to PBS (vehicle, 20 μL) with no response.

FIG. 1D and FIG. 1E depict graphs of fluorescence of CHO and BHK cells, respectively expressing human CCR7 at varying concentration of serum from immunized mice, demonstrating that a high titer in response to the number of TPM injections performed according to the immunization protocol.

FIG. 1F depicts a graph of fluorescence of CHO and BHK cells expressing human CCR7 and CHO parental cells at varying concentration of serum from best-responding mouse (Group20/#2) immunized with for the CCR7 Target Presentation Material in the form of CCR7-naked particles of this invention. The difference between binding of serum proteins to human CCR7-expressing cells is substantially and significantly higher than to parental cells, indicating that serum contains antibodies against human CCR7, which is further corroborated by comparison of binding to these cells by control animal derived serum:

FIG. 1G depicts a graph of fluorescence of CHO cells expressing human CCR7 vs. CHO parental cells (CHO Host) in the presence of the Serum (at 1/100 dilution) from the best mouse responder (Group20/#2) to immunization with the CCR7-naked particles of this invention, as compared to fluorescence of these cells in the presence of Serum (at the same dilution) from a control mouse immunized with vehicle (Group Control 20/#1). These data clearly show that TPM provide a powerful immunogen that elicit a robust immune response.

### Example 8

### Methods for Selection of Fully Human Antibodies

Phage-display libraries are among the most used technologies for generation and optimization of fully human antibodies (see Hoogenboom, H. R. Selecting and screening recombinant antibody libraries. Nature Biotechnol. 23, 1105-1116 (2005); Bradbury, A. R. & Marks, J. D. Antibodies from phage antibody libraries. J. Immunol. Methods 290, 29-49 (2004); and Fredericks, Z. L. et al. Identification of potent human anti-IL-1R I antagonist antibodies. Protein Eng. Des. Sel. 17, 95-106 (2004)). Other display technologies useful for the generation and affinity maturation (optimization) include yeast-, mRNA- and ribosome-display libraries—are gaining in popularity for selection and optimization of antibodies (see Hoogenboom, Id., Bradbury, Id., and Fredericks, Id.).

Display libraries display single-chain variable-domain antibody fragments (scFvs) or Fabs, and contain the encoding DNA or RNA. They have high genetic diversity or repertoire size (commonly 10⁹-10¹³). These technologies allow the selective recovery of clones that bind a target antigen from a library, and they provide the means to amplify the selected clones for further rounds of selection or analysis. The genetic diversity in these libraries is commonly created by cloning the repertoire of the immunoglobulin heavy-chain (HI) and and light-chain (VI) variable gene segments from naive or immunized individuals. Alternatively, this diversity can be achieved by using synthetic DNA to randomize the complementarity-determining regions (“CDRs”, the antigen-binding loops) or by a combination of these two approaches. The binding step can be undertaken with the target in solution, immobilized on a surface or on cells. After extensive washing, specifically bound clones are recovered and amplified for the next round of selection.

In some embodiments of this invention, anti-CCR7 antibodies are in IgG1 format. However, IgG2, IgG3, and IgG4 formats can also be used.

Once binders in the form of scFv- or Fab-carrying phage or phagemid particles are obtained from a respective library, they can be expressed in bacterial cells as individual antibody fragment proteins and purified. The purified antibody fragment proteins can be then characterized in terms of their affinity toward CCR7, specificity of binding to the target as compared to other GPCRs, functionality (capacity to inhibit CCL19 or CCL21-induced Ca-flux or chemotaxis), cross-reactivity with CCR7 mouse and cynomolgus monkey orthologs. Then genes encoding best antibody fragments can be converted into fully human IgGs by means of well-known in the art molecular biology procedures. The DNA vectors for heavy and light chains of the IgG antibodies so obtained can be used for transfection of CHO or other suitable mammalian cells for expressing fully human antibody against CCR7 in an IgG format.

### Example 9

### Human Antibodies Obtained by Immunization of Transgenic Mice

The generation of human antibodies by immunization of mice that are transgenic for human immunoglobulin genes and have disrupted mouse immunoglobulin heavy-chain and Igκ light-chain loci was first described in 1994. Subsequent progress included the expression of more V gene segments by the transgenic mice, thereby expanding the potential repertoire of recovered antibodies. Mouse strains that encode human antibodies with different heavy-chain isotypes have also been created to tailor effector functions. One problem in the generation of human antibodies for multispanning membrane proteins, such as G-protein coupled receptors (GPCRs), ion channels and transporters is that these proteins have a high rate of homology with the mouse protein, thus the animal immune system tolerance has to be broken. In addition, preparation of the native immunogen (better if purified) in the amounts required for the immunization may be a problem in the case of multispanning membrane proteins, although this problem may be overcome with the use of synthetic peptides and fusion proteins mimicking the fragments of the multispanning membrane proteins. However, the antibodies generated using this last method rarely appear with desirable neutralizing (antagonistic) properties.

### Example 10

### Purification of Anti-Human CCR7 Antibodies as scFv's or Fabs

Antibodies (in the form of scFv's or FABS that have his-tag) were purified using his-tag affinity purification protocol, as provided for scFv as follows. Each of the E. coli clones carrying phagemid with an anti-hCCR7 scFv gene was grown in 2×TY medium supplemented with 100 μg/ml ampicillin and 2% glucose at 37° C., 250 rpm to saturation, and each of the cultures so produced was used to inoculate 6 vessels each containing 50 ml 2×TY media supplemented with 100 μg/ml ampicillin and 0.1% glucose. The total volume for each scFv culture thus was 300 ml. Upon reaching logarithmic phase of growth (OD˜0.6) at 37° C., 250 rpm, IPTG was added to bacterial cultures to a final concentration of 0.05 mM. Cultures were incubated overnight at 30° C., 250 rpm.

In the morning, to recover csFv's accumulated in the periplasm, the cultures were centrifuged in 50 ml tubes at 2,500 rpm for 20 min using Beckman table-top centrifuge and the supernatant discarded. Each bacterial pellet (˜500 microliter) was re-suspended in 1,200 μliter ice-cold TES buffer containing 20% sucrose, 1 mM EDTA, 50 mM Tris-HCl, pH8.0, incubated on ice for 30 min, and then 800 μl of ice-cold 10 mM Tris-HCl, pH7.5 was added the mixture was then incubated on ice for another 30 min. All further purification procedures were performed at 4° C.

After centrifugation in 50 ml tubes at 2,500 rpm for 20 min in Beckman table-top centrifuge, the supernatants were collected into 2 ml Eppendorf tubes, clarified by centrifugation for 20 min at 14,000 rpm using Eppendorf table top refrigerated centrifuge, and the clarified supernatant was transferred into fresh 2 ml Eppendorf tubes (each tube contained thus ˜1,900 microliter clarified periplasm material in approximately half-diluted TES buffer).

After pre-washing Ni-agarose resin (High-Density IDA-Agarose 6 BCL Nickel Charged Resin (ABT)) twice in the Bind/Wash Buffer (300 mM NaCl, 20 mM Imidazol, 50 mM Tris-HCl, pH7.5, 0.05% Tween-20), a 100 microliter aliquot of the Ni-resin was added to each tube. Then each tube was incubated on a rotator for 3 h, centrifuged for 20 min at 2,000 rpm using an Eppendorf table top refrigerated centrifuge, and supernatant removed. The resin pellets of each scFv was combined in a fresh 2 ml Eppendorf tube (thus, scFv samples were obtained, each in the form of ˜600 μL Ni-resin carrying his-tag immobilized scFv).

Each sample was re-suspended in 1,400 microliter Bind/Wash buffer and incubated on a rotator for 40 min, centrifuged for 20 min at 2,000 rpm using an Eppendorf table top refrigerated centrifuge, and the supernatant was removed. To each of the resulting pellets (Ni-resin beads with scFv) a 600-microliter aliquot of the Elute. Buffer (20 mM EDTA, 100 mM NaCl, 20 mM Tris-HCl, pH7.5, 0.05% Tween-20) was added, and after incubation with rotation for 40 min and centrifuged for 20 min at 14,000 rpm using an Eppendorf table top refrigerated centrifuge, the supernatants (500 microliters each) were collected and supplemented with MgSO₄ added to final concentration 21 mM.

Generally similar protocols for Fab purification were employed.

### Example 11

### Isolation and Characterization of Binding of Anti-CCR7 Antibodies

Anti-CCR7 antibodies of this invention were produced in transiently transfected CHO cells in serum-free medium in IgG1 format and harvested on day 5 or 6 according to the protocol licensed from Canadian Research Council and purify under endotoxin-free condition using affinity chromatography on Protein A, acidic elution followed by immediate neutralization to pH6.0 and dialysis against a storage buffer. According to the SDS-PAGE, so purified antibodies were 0.95% pure, size-exclusion chromatography of antibody samples confirmed that each of so purified IgG1 antibodies contained less than 2% aggregates, and LAL-test detected endotoxins at the level below 1 endotoxin unit per 1 mg of protein.

Seven histograms are shown demonstrating the signal collected from the cells expressing human CCR7 and different other GPCRs (used as controls). Cells have been incubated with fully human anti-human CCR7 antibodies in IgG1 format. After washing out residual unbound antibodies the cells have been incubated with commercial anti-human Fe antibodies conjugated to the fluorescent dye phycoerythrin (IgG-PE). In more details, the cell staining procedure was: 5000-1000 cell suspension in 10 μl FACS buffer (1×PBS, 2.0% FBS, 0.2% sodium azide) was mixed with 10 μl of 200 nM the corresponding anti-CCR7 MAB and incubated on ice for 30 min. Washing step—after the incubation 150 μl of FACS buffer was added to the cell sample, the samples were mixed gently by up-down pipetting the cell suspension. Then the samples were centrifuged at 1100 rpm for 5 minutes, and supernatants were removed. Washing step was repeated ones. Then 10 μl of anti-human PE-(Fab)2 form Jackson Immuno Research Lab. #709-116-098 diluted 40 times in FACS buffer were added to the cells and the cells were re-suspended by up-down pipetting. After 20 min. incubation on ice in dark the washing step was repeated twice. The washed cells were mixed with 100 μl of FIX buffer (0.5% Paraformaldehyde solution in PBS). Fixed samples were stored in dark on ice and analyzed by FACS on Guava PCA-96 flow cytometer. The fluorescent signals collected from individual cells were obtained using fluorescence Activated Cell Sorter (FACS).

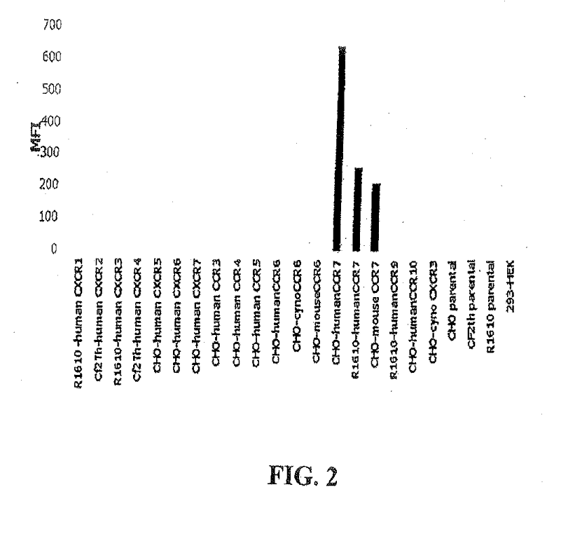

FIG. 2 depicts a graph of fluorescence of cells expressing CCR7 and other GPCRs labeled with human antibody IgG1 MSM-R707 of this invention. Columns 1-19 show results for the cells: (1) R1610-humanCXCR1; (2) Cf2th-humanCXCR2; (3) R1610-humanCXCR3; (4) Cf2th-humanCXCR4; (5) CHO-humanCXCR5; (6) CHO-humanCXCR6; (7) CHO-humanCXCR7; (8) CHO-humanCCR3; (9) CHO-humanCCR4; (10) CHO-humanCCR5; (11) CHO-humanCCR6; (12) CHO-cynoCCR6; (13) CHO-mouseCCR6; (14) CHO-humanCCR7; (15) R1610-humanCCR7; (16) CHO-mouseCCR7; (17) R1610-humanCCR9; (18) CHO-humanCCR10; and, (19) CHO-cynoCXCR3, respectively.

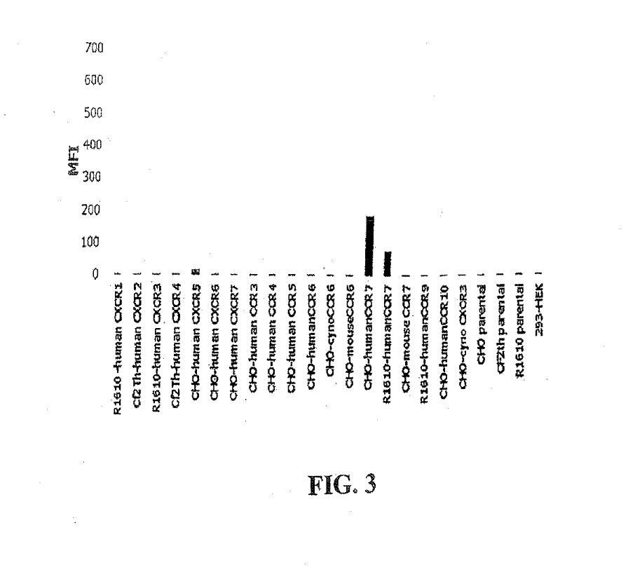

FIG. 3 depicts a graph of fluorescence of cells as in FIG. 2 expressing CCR7 or other GPCRs, and labeled with human antibody IgG1 MSM-R707B of this invention.

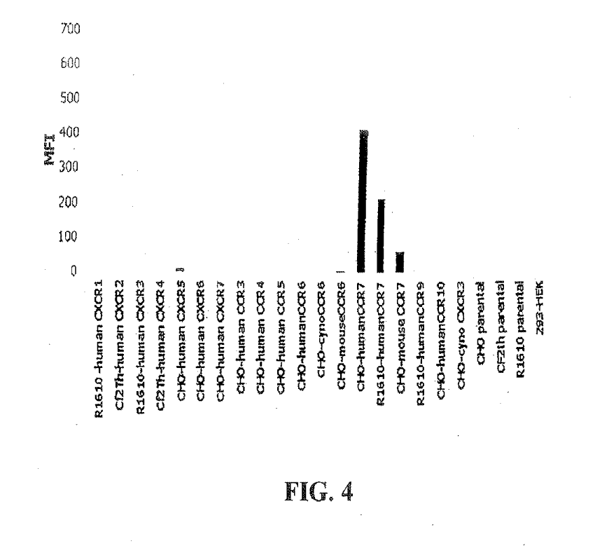

FIG. 4 depicts a graph of fluorescence of cells as in FIG. 2 expressing CCR7 or other GPCRs, and labeled with human antibody IgG1 MSM-R707BR of this invention.

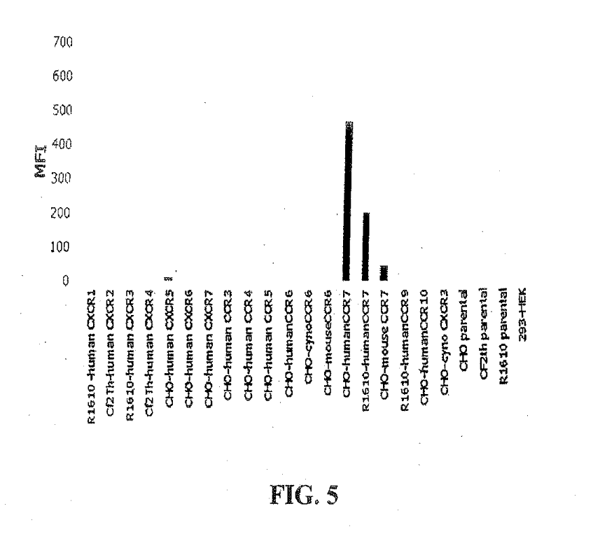

FIG. 5 depicts a graph of fluorescence of cells as in FIG. 2 expressing CCR7 or other GPCRs, and labeled with human antibody IgG1 MSM-R707BL of this invention.

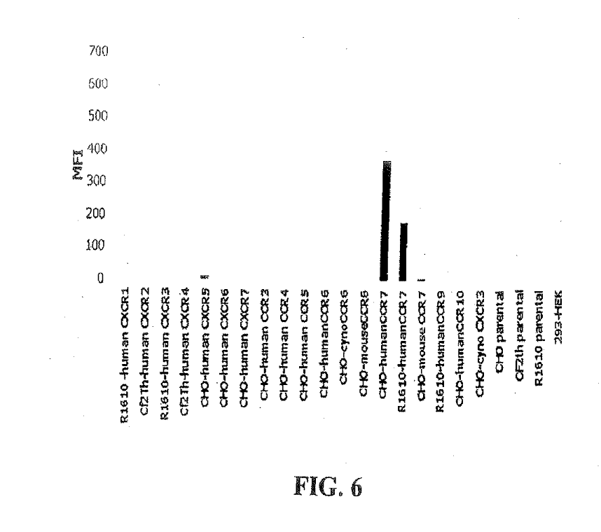

FIG. 6 depicts a graph of fluorescence of cells as in FIG. 2 expressing CCR7 or other GPCRs, and labeled with human antibody IgG1 MSM-R7707BI of this invention.

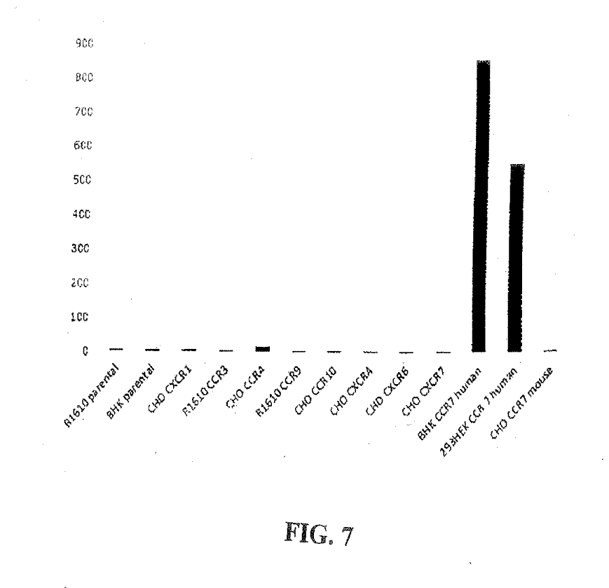

FIG. 7 depicts a graph of fluorescence of cells as in FIG. 2 expressing CCR7 or other GPCRs, and labeled with human antibody IgG1 MSM-R710 of this invention.

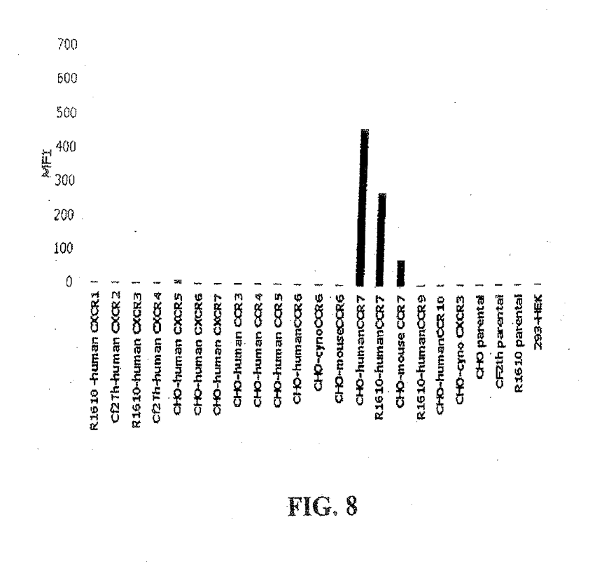

FIG. 8 depicts a graph of fluorescence of cells as in FIG. 2 expressing CCR7 or other GPCRs, and labeled with human antibody IgG1 MSM-R735 of this invention.

The data shown in FIGS. 2-8 demonstrate that all seven CCR7 IgG1 antibody clones selectively bind to human CCR7 but not to other G-protein coupled receptors. Several IgG1 antibodies of this invention display also binding to the mouse CCR7 ortholog, which can be advantageous for exploring the role of CCR7 in a broader range of mouse models of human diseases, as well as in validating novel animal models. Mouse and human CCR7 molecules have a high degree of homology and the cross-reactivity has been both expected and desired, because the human-mouse cross-reactivity helps in the following animal based evaluation studies on the antibodies for selection of antibody candidates for therapeutics development.

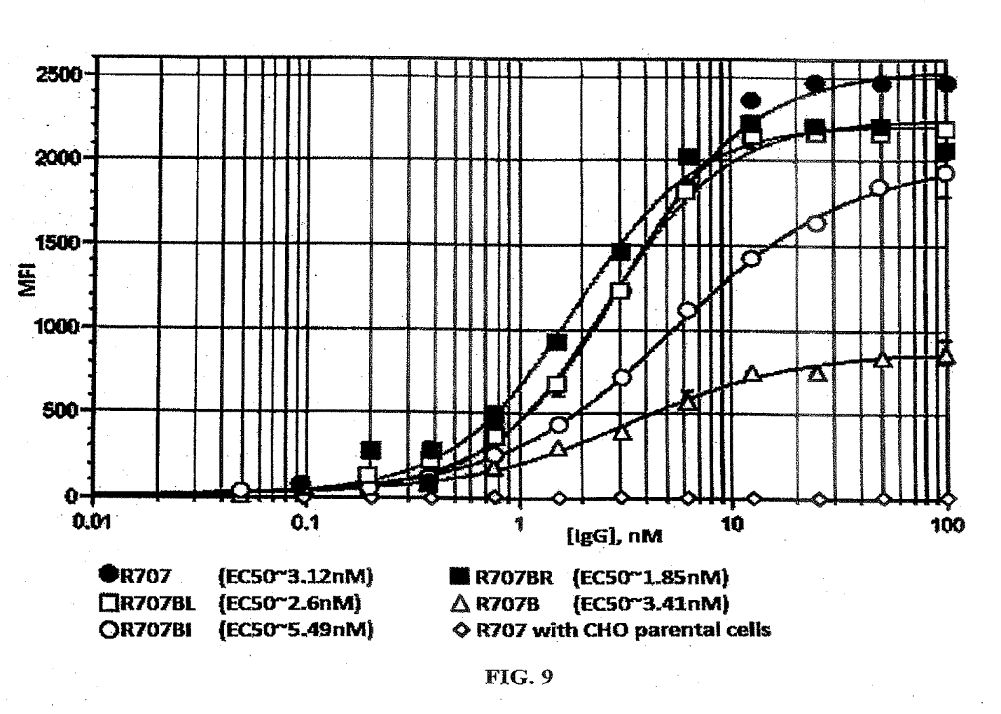

The affinity of IgG1 antibodies to human CCR7 was evaluated by measuring mean fluorescence value (MFI) of cells expressing CCR7 in the presence of varying concentration of antibody in question, upon staining of bound to the cells antibodies with a commercial anti-human antibody-PE conjugate. The data of such experiments are depicted in FIGS. 9, 10, and 11. In these experiments binding of some antibodies to parental cells or cells expressing mouse CCR7 was also quantitatively characterized by obtaining EC₅₀ value (a concentration of IgG1 at which half-maximum MFI is achieved) for each curve using the SoftMaxPro5 program.

The EC₅₀ values for human CCR7 binding of the antibodies of this invention were in sub-nanomolar to a few nanomolar range. FIG. 9 depicts a graph of fluorescence of CHO cells expressing human CCR7 in the presence of varying concentration of IgG1 antibodies of this invention, along with EC₅₀ values for MSM-R707 (EC₅₀=3.2 nM), MSM-R707BL (2.6 nM), MSM-R707BI (5.5 nM), MSM-R707BR (1.9 nM), and MSM-R707B (3.4 nM), and labeled with a commercial anti-human Fc PE-conjugate (as compared with CHO-parental cells for one of the antibodies, MSM-R707).

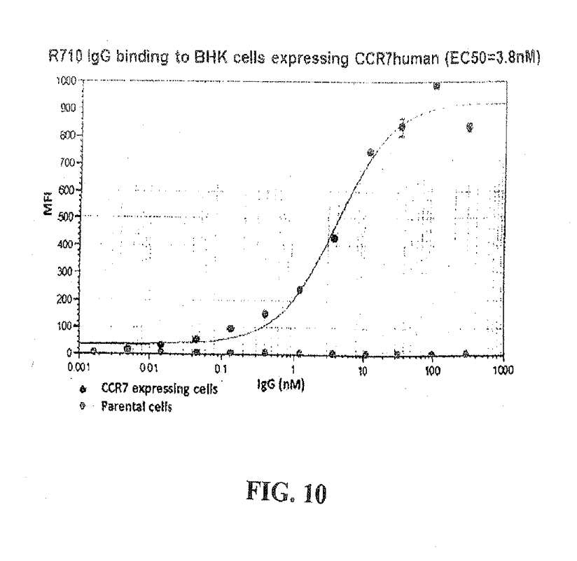

FIG. 10 depicts a graph of fluorescence of BHK cells expressing human CCR7 and BHK parental cells in the presence of varying concentration of IgG1 antibodies of this invention MSM-R710, for which EC₅₀ value of 3.8 nM was obtained.

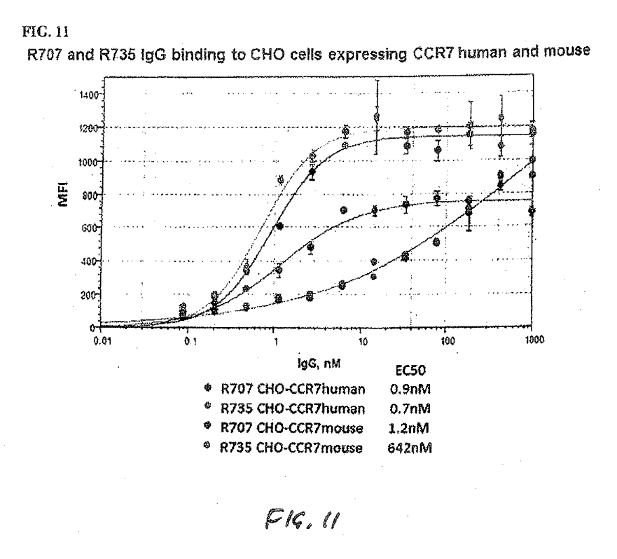

FIG. 11 depicts a graph of fluorescence of CHO cells expressing either human CCR7 or mouse CCR7 in the presence of varying concentration of IgG1 antibodies of this invention MSM-R707 (for CHO-human CCR7 cells data of another experiment that shown in FIG. 9 are provided) or MSM-R735, which displayed EC₅₀ values for human CCR7 of 0.9 nM and 0.7 nM, respectively. The value of EC₅₀ in sub-nanomolar to a few nM range is typically sufficient to exert a therapeutic effect at reasonable concentration. Also, EC₅₀ for the mouse CCR7 ortholog was evaluated in this experiment (FIG. 11): For MSM-R707 it was found to be 1.2 nM, whereas much worse affinity to the ortholog of MSM-R735 was observed (EC₅₀˜642 nM).

### Example 12

### Sequencing of Antibody Fragments I

Fully human antibodies depicted in FIGS. 2-14 were sequenced. Tables 3 through 9 show sequence data for each of 7 clones for which cell binding data is presented. Each of the clones has a unique sequence and thus the Tables do not include duplications. For each antibody has sequences of heavy chain variable domain in DNA format (VH DNA) and of light chain in DNA format (VL DNA), the same sequences in amino acid format (HC AA and LC AA, respectively), as well as amino acid sequences of 1^{st}, 2^{nd}, and 3^{rd} CDRs of heavy chain (CDR1 HC AA, CDR2 HC AA, CDR3 HC AA) and of light chain (CDR1 LC AA, CDR2 LC AA, CDR3 LC AA). The antibodies in Tables 5-11 are in IgG1 format. Other antibodies in IgG4 format are shown below in Tables 14 and 15.

TABLE 5
Sequences of Anti-CCR7 Antibody MSM R707

| Sequence | Sequence of | |
|---|---|---|
| Id No: | IgG 1: | Sequence |
| SEQ ID NO: 3 | VH DNA | GAAGTTCAACTGCTGGAGTCCGGTGGTGGTCTG |
| | | GTACAGCCGGGTGGTTCTCTGCGTCTGAGTTGCG |
| | | CGGCCAGTGGCTTTACCTTCAGTAACTATGCGAT |
| | | CCATTGGGTGCGTCAGGCTCCGGGCAAAGGTCT |
| | | GGAATGGGTTAGCGCTATTACTCCGAGGGGTGG |
| | | CTATACCTACTATGCGGATAGCGTGAAAGGCCGT |
| | | TTTACCATTTCTCGCGACAACAGCAAGAACACGC |
| | | TGTACCTGCAGATGAACTCACTGCGTGCCGAAGA |
| | | TACGGCCGTGTATTACTGTGCGAGAGGCCTGACG |
| | | atgatgTACACTCCCGGCatgGACTACTGGGGCCAGGG |
| | | AACCTTGGTCACCGTCTCGAGT |
| SEQ ID NO: 4 | VL DNA | GAAATTGTGCTGACCCAGTCTCCGGGCACGTTAT |
| | | CTCTGAGCCCTGGTGAGCGCGCCACTCTGTCATG |
| | | CCGGGCTTCTCAAAGTGTTAGCAGTAGCTACCTG |
| | | GCGTGGTATCAGCAAAAACCGGGCCAGGCCCCGC |
| | | GTCTGCTGATTTACGGTGCATCCAGCCGTGCCACC |
| | | GGCATTCCAGATCGTTTTTCCGGTAGTGGTTCTGG |
| | | GACGGACTTCACTCTGACAATCTCACGCCTGGAA |
| | | CCGGAGGATTTTGCGGTGTATTACTGCCAGCAAT |
| | | CTTATTCTTCTCCTATCACGTTCGGCCAAGGGACC |
| | | AAGGTGGAAATCAAA |
| SEQ ID NO: 5 | HC AA | EVQLLESGGGLVQPGGSLRLSCAASGFTFSNYAIHW |
| | | VRQAPGKGLEWVSAITPRGGYTYYADSVKGRFTISR |
| | | DNSKNTLYLQMNSLRAEDTAVYYCARGLTMMYTPG |
| | | MDYWGQGTLVTVSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVD |
| | | KRVEPKSCDKTHTCPPCPAPELLGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYK |
| | | CKVSNKALPAPIEKTISKAKGQPREPQVYTLPPSREE |
| | | MTKNQVSLTCLVKGFYPSDIAVEWESNGQPENNYK |
| | | TTPPVLDSDGSFFLYSKLTVDKSRWQQGNVFSCSV |
| | | MHEALHNHYTQKSLSLSPGK* |
| SEQ ID NO: 6 | LC AA | EIVLTQSPGTLSLSPGERATLSCRASQSVSSSYLAWY |
| | | QQKPGQAPRLLIYGASSRATGIPDRFSGSGSGTDFTLT |
| | | ISRLEPEDFAVYYCQQSYSSPITFGQGTKVEIKRTVAAP |
| | | SVFIFPPSDEQLKSGTASVVCLLNNFYPREAKVQWKV |
| | | DNALQSGNSQESVTEQDSKDSTYSLSSTLTLSKADYE |
| | | KHKVYACEVTHQGLSSPVTKSFNRGEC* |
| SEQ ID NO: 7 | CDR1 HC AA | NYAIH |
| SEQ ID NO: 8 | CDR2 HC AA | AITPRGGYTYYADSVKG |
| SEQ ID NO: 9 | CDR3 HC AA | GLTMMYTPGMDY |
| SEQ ID NO: 10 | CDR1 LC AA | RASQSVSSSYLA |
| SEQ ID NO: 11 | CDR2 LC AA | GASSRAT |
| SEQ ID NO: 12 | CDR3 LC AA | QQSYSSPIT |

TABLE 6
Sequences of Anti-CCR7 Antibody MSM R707B

| Sequence | Sequence | |
|---|---|---|
| Id No: | of IgG 1: | Sequence |
| SEQ ID | VH DNA | GAAGTTCAACTGCTGGAGTCCGGTGGTGGTCTGGTACAGCC |
| NO: 13 | | GGGTGGTTCTCTGCGTCTGAGTTGCGCGGCCAGTGGCTTTAC |
| | | CTTCAGTAACTATGCGATCCATTGGGTGCGTCAGGCTCCGGG |
| | | CAAAGGTCTGGAATGGGTTAGCGCTATTACTCCGAGGGGTG |
| | | GCTATACCTACTATGCGGATAGCGTGAAAGGCCGTTTTACCA |
| | | TTTCTCGCGACAACAGCAAGAACACGCTGTACCTGCAGATGAACT |
| | | CACTGCGTGCCGAAGATACGGCCGTGTATTACTGTGCGAGAGGCC |
| | | TGACGtactctTACACTCCCGGCtTtGACTACTGGGGCCAGGGAACCTT |
| | | GGTCACCGTCTCGAGT |
| SEQ ID | VL DNA | GAAATTGTGCTGACCCAGTCTCCGGGCACGTTATCTCTGAGC |
| NO: 14 | | CCTGGTGAGCGCGCCACTCTGTCATGCCGGGCTTCTCAAAGT |
| | | GTTAGCAGTAGCTACCTGGCGTGGTATCAGCAAAAACCGGGCCAG |
| | | GCCCCGCGTCTGCTGATTTACGGTGCATCCAGCCGTGCCACCGGC |
| | | ATTCCAGATCGTTTTTCCGGTAGTGGTTCTGGGACGGACTTCACTC |
| | | TGACAATCTCACGCCTGGAACCGGAGGATTTTGCGGTGTATTACTG |
| | | CCAGCAATCTTATTCTTCTCCTATCACGTTCGGCCAAGGGACCAAG |
| | | GTGGAAATCAAA |
| SEQ ID | HC AA | EVQLLESGGGLVQPGGSLRLSCAASGFTFSNYAIHWVRQAPGKGLE |
| NO: 15 | | WVSAITPRGGYTYYADSVKGRFTISRDNSKNTLYLQMNSLRAEDTAV |
| | | YYCARGLTYSYTPGFDYWGQGTLVTVSSASTKGPSVFPLAPSSKSTS |
| | | GGTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPCPA |
| | | PELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQ |
| | | GNVFSCSVMHEALHNHYTQKSLSLSPGK* |
| SEQ ID | LC AA | EIVLTQSPGTLSLSPGERATLSCRASQSVSSSYLAWYQQKPGQAPRLLI |
| NO: 16 | | YGASSRATGIPDRFSGSGSGTDFTLTISRLEPEDFAVYYCQQSYSSPITF |
| | | GQGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVY |
| | | ACEVTHQGLSSPVTKSFNRGEC* |
| SEQ ID | CDR1 HC | NYAIH |
| NO: 17 | AA | |
| SEQ ID | CDR2 HC | AITPRGGYTYYADSVKG |
| NO: 18 | AA | |
| SEQ ID | CDR3 HC | GLTYSYTPGFDY |
| NO: 19 | AA | |
| SEQ ID | CDR1 LC | RASQSVSSSYLA |
| NO: 20 | AA | |
| SEQ ID | CDR2 LC | GASSRAT |
| NO: 21 | AA | |
| SEQ ID | CDR3 LC | QQSYSSPIT |
| NO: 22 | AA | |

TABLE 7
Sequences of Anti-CCR7 Antibody MSM R707BR

| Sequence | Sequence | |
|---|---|---|
| Id No: | of IgG 1: | Sequence |
| SEQ ID | VH DNA | GAAGTTCAACTGCTGGAGTCCGGTGGTGGTCTGGTACAG |
| NO: 23 | | CCGGGTGGTTCTCTGCGTCTGAGTTGCGCGGCCAGTGGCT |
| | | TTACCTTCAGTAACTATGCGATCCATTGGGTGCGTCAGGC |
| | | TCCGGGCAAAGGTCTGGAATGGGTTAGCGCTATTACTCCG |
| | | AGGGGTGGCTATACCTACTATGCGGATAGCGTGAAAGGCC |
| | | GTTTTACCATTTCTCGCGACAACAGCAAGAACACGCTGTA |
| | | CCTGCAGATGAACTCACTGCGTGCCGAAGATACGGCCGT |
| | | GTATTACTGTGCGAGAGGCCTGACGcgctctTACACTCCCGG |
| | | CtTtGACTACTGGGGCCAGGGAACCTTGGTCACCGTCTCG |
| | | AGT |
| SEQ ID | VL DNA | GAAATTGTGCTGACCCAGTCTCCGGGCACGTTATCTCTGA |
| NO: 24 | | GCCCTGGTGAGCGCGCCACTCTGTCATGCCGGGCTTCTCA |
| | | AAGTGTTAGCAGTAGCTACCTGGCGTGGTATCAGCAAAAA |
| | | CCGGGCCAGGCCCCGCGTCTGCTGATTTACGGTGCATCCAG |
| | | CCGTGCCACCGGCATTCCAGATCGTTTTTCCGGTAGTGGTT |
| | | CTGGGACGGACTTCACTCTGACAATCTCACGCCTGGAACC |
| | | GGAGGATTTTGCGGTGTATTACTGCCAGCAATCTTATTCTT |
| | | CTCCTATCACGTTCGGCCAAGGGACCAAGGTGGAAATCA |
| | | AA |
| SEQ ID | HC AA | EVQLLESGGGLVQPGGSLRLSCAASGFTFSNYAIHWVRQAPG |
| NO: 25 | | KGLEWVSAITPRGGYTYYADSVKGRFTISRDNSKNTLYLQMN |
| | | SLRAEDTAVYYCARGLTRSYTPGFDYWGQGTLVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKRVEPKSCDKTHTCPPCPAPELLGGPSVFLFPPKPKDTLMI |
| | | SRTPEVTCVVVDVSHEDPEVKFNWYVDGVEVHNAKTKPREE |
| | | QYNSTYRVVSVLTVLHQDWLNGKEYKCKVSNKALPAPIEKTI |
| | | SKAKGQPREPQVYTLPPSREEMTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPGK* |
| SEQ ID | LC AA | EIVLTQSPGTLSLSPGERATLSCRASQSVSSSYLAWYQQKPGQA |
| NO: 26 | | PRLLIYGASSRATGIPDRFSGSGSGTDFTLTISRLEPEDFAVYYCQ |
| | | QSYSSPITFGQGTKVEIKRTVAAPSVFIFPPSDEQLKSGTASVVCL |
| | | LNNFYPREAKVQWKVDNALQSGNSQESVTEQDSKDSTYSLSST |
| | | LTLSKADYEKHKVYACEVTHQGLSSPVTKSENRGEC* |
| SEQ ID | CDR1 HC | NYAIH |
| NO: 27 | AA | |
| SEQ ID | CDR2 HC | AITPRGGYTYYADSVKG |
| NO: 28 | AA | |
| SEQ ID | CDR3 HC | GLTRSYTPGFDY |
| NO: 29 | AA | |
| SEQ ID | CDR1 LC | RASQSVSSSYLA |
| NO: 30 | AA | |
| SEQ ID | CDR2 LC | GASSRAT |
| NO: 31 | AA | |
| SEQ ID | CDR3 LC | QQSYSSPIT |
| NO: 32 | AA | |

TABLE 8
Sequences of Anti-CCR7 Antibody MSM R707BL

| Sequence | Sequence | |
|---|---|---|
| Id No: | of IgG1: | Sequence |
| SEQ ID | VH DNA | GAAGTTCAACTGCTGGAGTCCGGTGGTGGTCTGGTACAGCC |
| NO: 33 | | GGGTGGTTCTCTGCGTCTGAGTTGCGCGGCCAGTGGCTTTAC |
| | | CTTCAGTAACTATGCGATCCATTGGGTGCGTCAGGCTCCGGG |
| | | CAAAGGTCTGGAATGGGTTAGCGCTATTACTCCGAGGGGTG |
| | | GCTATACCTACTATGCGGATAGCGTGAAAGGCCGTTTTACCA |
| | | TTTCTCGCGACAACAGCAAGAACACGCTGTACCTGCAGATG. |
| | | AACTCACTGCGTGCCGAAGATACGGCCGTGTATTACTGTGC |
| | | GAGAGGCCTGACGctgtctTACACTCCCGGCtTtGACTACTGGGG |
| | | CCAGGGAACCTTGGTCACCGTCTCGAGT |
| SEQ ID | VL DNA | GAAATTGTGCTGACCCAGTCTCCGGGCACGTTATCTCTGAG |
| NO: 34 | | CCCTGGTGAGCGCGCCACTCTGTCATGCCOGGCTTCTCAAA |
| | | GTGTTAGCAGTAGCTACCTGGCGTGGTATCAGCAAAAACCG |
| | | GGCCAGGCCCCGCGTCTGCTGATTTACGGTGCATCCAGCCG |
| | | TGCCACCGGCATTCCAGATCGTTTTFCCGGTAGTGGTTCTGG |
| | | GACGGACTTCACTCTGACAATCTCACGCCTGGAACCGGAGG |
| | | ATTTTGCGGTGTATTACTGCCAGCAATCTTATTCTTCTCCTA |
| | | TCACGTTCGGCCAAGGGACCAAGGTGGAAATCAAA |
| SEQ ID | HC AA | EVQLLESGGGLVQPGGSLRLSCAASGFTFSNYAIHWVRQAPGK |
| NO: 35 | | GLEWVSAITPRGGYTYYADSVKGRFTISRDNSKNTLYLQMNSL |
| | | RAEDTAVYYCARGLTLSYTPGFDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISRT |
| | | PEVTCVVVDVSHEDPEVKFNWYVDGVEVHNAKTKPREEQYNS |
| | | TYRVVSVLTVLHQDWLNGKEYKCKVSNKALPAPIEKTISKAKG |
| | | QPREPQVYTLPPSREEMTKNQVSLTCLVKGFYPSDIAVEWESNG |
| | | QPENNYKTTPPVLDSDGSFFLYSKLTVDKSRWQQGNVFSCSVM |
| | | HEALHNHYTQKSLSLSPGK* |
| SEQ ID | LC AA | EIVLTQSPGTLSLSPGERATLSCRASQSVSSSYLAWYQQKPGQAP |
| NO: 36 | | RLLIYGASSRATGIPDRFSGSGSGTDFTLTISRLEPEDFAVYYCQQ |
| | | SYSSPITFGQGTKVEIKRTVAAPSVFIFPPSDEQLKSGTASVVCLL |
| | | NNFYPREAKVQWKVDNALQSGNSQESVTEQDSKDSTYSLSSTL |
| | | TLSKADYEKHKVYACEVTHQGLSSPVTKSFNRGEC* |
| SEQ ID | CDR1 HC | NYAIH |
| NO: 37 | AA | |
| SEQ ID | CDR2 HC | AITPRGGYTYYADSVKG |
| NO: 38 | AA | |
| SEQ ID | CDR3 HC | GLTLSYTPGFDY |
| NO: 39 | AA | |
| SEQ ID | CDR1 LC | RASQSVSSSYLA |
| NO: 40 | AA | |
| SEQ ID | CDR2 LC | GASSRAT |
| NO: 41 | AA | |
| SEQ ID | CDR3 LC | QQSYSSPIT |
| NO: 42 | AA | |

TABLE 9
Sequences of Anti-CCR7 Antibody MSM R707BI

| Sequence | Sequence of | |
|---|---|---|
| Id No: | IgG1: | Sequence |
| SEQ ID NO: 43 | VH DNA | GAAGTTCAACTGCTGGAGTCCGGTGGTGGTCTGGTACAG |
| | | CCGGGTGGTTCTCTGCGTCTGAGTTGCGCGGCCAGTGGCT |
| | | TTACCTTCAGTAACTATGCGATCCATTGGGTGCGTCAGGC |
| | | TCCGGGCAAAGGTCTGGAATGGGTTAGCGCTATTACTCC |
| | | GAGGGGTGGCTATACCTACTATGCGGATAGCGTGAAAGG |
| | | CCGTTTTACCATTTCTCGCGACAACAGCAAGAACACGCT |
| | | GTACCTGCAGATGAACTCACTGCGTGCCGAAGATACGG |
| | | CCGTGTATTACTGTGCGAGAGGCCTGACGatctctTACACT |
| | | CCCGGCtTtGACTACTGGGGCCAGGGAACCTTGGTCACC |
| | | GTCTCGAGT |
| SEQ ID NO: 44 | VL DNA | GAAATTGTGCTGACCCAGTCTCCGGGCACGTTATCTCTGA |
| | | GCCCTGGTGAGCGCGCCACTCTGTCATGCCGGGCTTCTCA |
| | | AAGTGTTAGCAGTAGCTACCTGGCGTGGTATCAGCAAAAA |
| | | CCGGGCCAGGCCCCGCGTCTGCTGATTTACGGTGCATCCAG |
| | | CCGTGCCACCGGCATTCCAGATCGTTTTTCCGGTAGTGGTT |
| | | CTGGGACGGACTTCACTCTGACAATCTCACGCCTGGAACC |
| | | GGAGGATTTTGCGGTGTATTACTGCCAGCAATCTTATTCTT |
| | | CTCCTATCACGTTCGGCCAAGGGACCAAGGTGGAAATCA |
| | | AA |
| SEQ ID NO: 45 | HC AA | EVQLLESGGGLVQPGGSLRLSCAASGFTFSNYAIHWVRQAP |
| | | GKGLEWVSAITPRGGYTYYADSVKGRFTISRDNSKNTLYLQ |
| | | MNSLRAEDTAVYYCARGLTISYTPGFDYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDKTHTCPPCPAPELLGGPSVFLFPPKP |
| | | KDTLMISRTPEVTCVVVDVSHEDPEVKFNWYVDGVEVHNA |
| | | KTKPREEQYNSTYRVVSVLTVLHQDWLNGKEYKCKVSNKA |
| | | LPAPIEKTISKAKGQPREPQVYTLPPSREEMTKNQVSLTCLVK |
| | | GFYPSDIAVEWESNGQPENNYKTTPPVLDSDGSFFLYSKLTV |
| | | DKSRWQQGNVFSCSVMHEALHNHYTQKSLSLSPGK* |
| SEQ ID NO: 46 | LC AA | EIVLTQSPGTLSLSPGERATLSCRASQSVSSSYLAWYQQKPG |
| | | QAPRLLIYGASSRATGIPDRFSGSGSGTDFTLTISRLEPEDFAV |
| | | YYCQQSYSSPITFGQGTKVEIKRTVAAPSVFIFPPSDEQLKSGT |
| | | ASVVCLLNNFYPREAKVQWKVDNALQSGNSQESVTEQDSK |
| | | DSTYSLSSTLTLSKADYEKHKVYACEVTHQGLSSPVTKSFNR |
| | | GEC* |
| SEQ ID NO: 47 | CDR1 HC AA | NYAIH |
| SEQ ID NO: 48 | CDR2 HC AA | AITPRGGYTYYADSVKG |
| SEQ ID NO: 49 | CDR3 HC AA | GLTISYTPGFDY |
| SEQ ID NO: 50 | CDRI LC AA | RASQSVSSSYLA |
| SEQ ID NO: 51 | CDR2 LC AA | GASSRAT |
| SEQ ID NO: 52 | CDR3 LC AA | QQSYSSPIT |

TABLE 10
Sequences of Anti-CCR7 Antibody MSM R710

| Sequence | Sequence of | |
|---|---|---|
| Id No: | IgGl: | Sequence |
| SEQ ID NO: 53 | VH DNA | GAAGTTCAACTGCTGGAGTCCGGTGGTGGTCTGGT |
| | | ACAGCCGGGTGGTTCTCTGCGTCTGAGTTGCGCGG |
| | | CCAGTGGCTTTACCTTCAGTAACTATACGATGCAT |
| | | TGGGTGCGTCAGGCTCCGGGCAAAGGTCTGGAAT |
| | | GGGTTAGCGGGATTGGTCCGAGGAGTGGCAGGAC |
| | | CTACTATGCGGATAGCGTGAAAGGCCGTTTTACCA |
| | | TTTCTCGCGACAACAGCAAGAACACGCTGTACCTG |
| | | CAGATGAACTCACTGCGTGCCGAAGATACGGCCGT |
| | | GTATTACTGTGCGAGATCTTACGCTTACCAGTACC |
| | | GTGGCTTCGACTACTGGGGCCAGGGAACCTTGGTC |
| | | ACCGTCTCGAGT |
| SEQ ID NO: 54 | VL DNA | GAAATTGTGCTGACCCAGTCTCCGGGCACGTTATCT |
| | | CTGAGCCCTGGTGAGCGCGCCACTCTGTCATGCCGG |
| | | GCTTCTCAAAGTGTTAGCAGTAGCTACCTGGCGTGG |
| | | TATCAGCAAAAACCGGGCCAGGCCCCGCGTCTGCTG |
| | | ATTTACGGTGCATCCAGCCGTGCCACCGGCATTCCA |
| | | GATCGTTTTTCCGGTAGTGG1TCTGGGACGGACTTCA |
| | | CTCTGACAATCTCACGCCTGGAACCGGAGGATTTTG |
| | | CGGTGTATTACTGCCAGTCTTCTGTCACGTTCGGCCA |
| | | AGGGACCAAGGTGGAAATCAAA |
| SEQ ID NO: 55 | HC AA | EVQLLESGGGLVQPGGSLRLSCAASGFTFSNYTMHW |
| | | VRQAPGKGLEWVSGIGPRSGRTYYADSVKGRFTISR |
| | | DNSKNTLYLQMNSLRAEDTAVYYCARSYAYQYRGF |
| | | DYWGQGTLVTVSSASTKGPSVFPLAPSSKSTSGGT |
| | | AALGCLVKDYFPEPVTVSWNSGALTSGVHTFPAVL |
| | | QSSGLYSLSSVVTVPSSSLGTQTYICNVNHKPSNTK |
| | | VDKRVEPKSCDKTHTCPPCPAPELLGGPSVFLFPPK |
| | | PKDTLMISRTPEVTCVVVDVSHEDPEVKFNWYVD |
| | | GVEVHNAKTKPREEQYNSTYRVVSVLTVLHQDW |
| | | LNGKEYKCKVSNKALPAPIEKTISKAKGQPREPQV |
| | | YTLPPSREEMTKNQVSLTCLVKGFYPSDIAVEWES |
| | | NGQPENNYKTTPPVLDSDGSFFLYSKLTVDKSRWQ |
| | | QGNVFSCSVMHEALHNHYTQKSLSLSPGK |
| SEQ ID NO: 56 | LC AA | EIVLTQSPGTLSLSPGERATLSCRASQSVSSSYLAWY |
| | | QQKPGQAPRLLIYGASSRATGIPDRFSGSGSGTDFT |
| | | LTISRLEPEDFAVYYCQSSVTFGQGTKVEIKRTVAA |
| | | PSVFIFPPSDEQLKSGTASVVCLLNNEYPREAKVQ |
| | | WKVDNALQSGNSQESVTEQDSKDSTYSLSSTLTLS |
| | | KADYEKHKVYACEVTHQGLSSPVTKSFNRGEC |
| SEQ ID NO: 57 | CDR1 HC AA | NYTMH |
| SEQ ID NO: 58 | CDR2 HC AA | GIGPRSGRTYYADSVKG |
| SEQ ID NO: 59 | CDR3 HC AA | SYAYQYRGFDY |
| SEQ ID NO: 60 | CDR1 LC AA | RASQSVSSSYLA |
| SEQ ID NO: 61 | CDR2 LC AA | GASSRAT |
| SEQ ID NO: 62 | CDR3 LC AA | QSSVT |

TABLE 11
Sequences of Anti-CCR7 Antibody MSM R735

| Sequence | Sequence of | |
|---|---|---|
| Id No: | IgG1: | Sequence |
| SEQ ID NO: 63 | VH DNA | GAAGTTCAACTGCTGGAGTCCGGTGGTGGTCTGGTAC |
| | | AGCCGGGTGGTCCTCTGCGTCTGAGTTGCGCGGCCAG |
| | | TGGCTTTACCTTCAGTAACTATAATATGCATTGGGTG |
| | | CGTCAGGCTCCGGGCAAAGGTCTGGAATGGGTTAGC |
| | | GGGATTGGGCCGCGTCGGGGCCGGACCTATTATGCG |
| | | GATAGCGTGAAAGGCCGTTTTACCATTTCTCGCGAC |
| | | AACAGCAAGAACACGCTGTACCTGCAGATGAACTCA |
| | | CTGCGTGCCGAAGATACGGCCGTGTATTACTGTGCG |
| | | AGATCTTACGCTTACCAGTACCGTGGCTTGGACTAC |
| | | TGGGGCCAGGGAACCCTGGTCACCGTCTCGAGT |
| SEQ ID NO: 64 | VL DNA | GAAATTGTGCTGACCCAGTCTCCGGGCACGTTATCTCT |
| | | GAGCCCTGGTGAGCGCGCCACTCTGTCATGCCGGGCTT |
| | | CTCAAAGTGTTAGCAGTAGCTACCTGGCGTGGTATCA |
| | | GCAAAAACCGGGCCAGGCCCCGCGTCTGCTGATTTAC |
| | | GGTGCATCCAGCCGTGCCACCGGCATTCCAGATCGT |
| | | TTTTCCGGTAGTGGTTCTGGGACGGACTTCACTCTGA |
| | | CAATCTCACGCCTGGAACCGGAGGATTTTGCGGTGTA |
| | | TTACTGCCAGCAAGGTAGTCCTGTCACGTTCGGCCAA |
| | | GGGACCAAGGTGGAAATCAAA |
| SEQ ID NO: 65 | HC AA | EVQLLESGGGLVQPGGSLRLSCAASGFTFSNYNMHWV |
| | | RQAPGKGLEWVSGIGPRRGRTYYADSVKGRFTISRDN |
| | | SKNTLYLQMNSLRAEDTAVYYCARSYAYQYRGLDYW |
| | | GQGTLVTVSSASTKGPSVFPLAPSSKSTSGGTAALGCL |
| | | VKDYFPEPVTVSWNSGALTSGVHTFPAVLQSSGLYSL |
| | | SSVVTVPSSSLGTQTYICNVNHKPSNTKVDKRVEPKS |
| | | CDKTHTCPPCPAPELLGGPSVFLEPPKPKDTLMISRTP |
| | | EVTCVVVDVSHEDPEVKFNWYVDGVEVHNAKTKPR |
| | | EEQYNSTYRVVSVLTVLHQDWLNGKEYKCKVSNKA |
| | | LPAPIEKTISKAKGQPREPQVYTLPPSREEMTKNQVSL |
| | | TCLVKGFYPSDIAVEWESNGQPENNYKTTPPVLDSDG |
| | | SFFLYSKLTVDKSRWQQGNVFSCSVMHEALHNHYT |
| | | QKSLSLSPGK |
| SEQ ID NO: 66 | LC AA | EIVLTQSPGTLSLSPGERATLSCRASQSVSSSYLAWY |
| | | QQKPGQAPRLLIYGASSRATGIPDRFSGSGSGTDFTL |
| | | TISRLEPEDFAVYYCQQGSPVTFGQGTKVEIKRTVAA |
| | | PSVFIFPPSDEQLKSGTASVVCLLNNFYPREAKVQWK |
| | | VDNALQSGNSQESVTEQDSKDSTYSLSSTLTLSKAD |
| | | YEKHKVYACEVTHQGLSSPVTKSENRGEC |
| SEQ ID NO: 67 | CDR1 HC AA | NYNMH |
| SEQ ID NO: 68 | CDR2 HC AA | GIGPRRGRTYYADSVKG |
| SEQ ID NO: 69 | CDR3 HC AA | SYAYQYRGLDY |
| SEQ ID NO: 70 | CDR1 LC AA | RASQSVSSSYLA |
| SEQ ID NO: 71 | CDR2 LC AA | GASSRAT |
| SEQ ID NO: 72 | CDR3 LC AA | QQGSPVT |

### Example 13

### Generation of R1610-hCCR7: Chinese Hamster Lung Fibroblasts Expressing Human CCR7

R1610-hCCR7 cells were obtained by transfecting the R1610 cells (Chinese Hamster. Lung Fibroblasts; ATCC, catalog number CRL1657) using Lipofectamin 2000 transfection reagent (Invitrogen, catalog number 11668019), according to the manufacturer's protocol, with the commercial pCMV-Script Vector™ (Catalog #212220, Stratagene) carrying a synthetic, mammalian cell expression optimized, human CCR7 gene (encodes the human CCR7 amino acid sequence of 378 amino acids; the Swissprot accession number P32248:

SEQ ID NO: 73
MDLGKPMKSVLVVALLVIFQVCLCQDEVTDDYIGDNTTVDYTLFESLCSK
KDVRNFKAWFLPIMYSIICFVGLLGNGLVVLTYIYFKRLKTMTDTYLLNL
AVADILFLLTLPFWAYSAAKSWVFGVHFCKLIFAIYKMSFFSGMLLLLCI
SIDRYVAIVQAVSAHRHRARVLLISKLSCVGIWILATVLSIPELLYSDLQ
RSSSEQAMRCSLITEHVEAFITIQVAQMVIGFLVPLLAMSFCYLVIIRTL
LQARNFERNKAIKVIIAVVVVFIVFQLPYNGVVLAQTVANFNITSSTCEL
SKQLNIAYDVTYSLACVRCCVNPFLYAFIGVKFRNDLFKLFKDLGCLSQE
QLRQWSSCRHIRRSSMSVEAETTTTFSP

The CCR7 coding region had a C-terminal extension of nucleotides that encode a two amino acid (S and A) linker followed by the Streptavidin-tag

SEQ ID NO: 74
MDEKTTGWRGGHVVEGLAGELEQLRARLEHHPQGQREP

followed by another two amino acid (GG) linker and the S-tag

SEQ ID NO: 75
KETAAAKFERQHMDS

The R1610 cells so transfected were cloned and CCR7 expressing clones were selected.

### Mechanisms of Action of Antibodies in Various Cancers

Table 12 below depicts some mechanisms of action of antibodies in cancers.

TABLE 12
Mechanisms of Action of Antibodies in Cancer Therapeutics

| | | | CCR7⁺ | Fibrocytes 10% |
|---|---|---|---|---|
| Antibody | Tregs CCR7⁺ | Dendritic CCR7⁺ | Metastatic Cells | CCR7⁺ |
| Simple | Stops chemotaxis | Stops chemotaxis | Stops chemotaxis | Stops chemotaxis |
| IgG4 Neutralizing | Neutralizes in vivo | Neutralizes in vivo | Neutralizes anti- | |
| | immune | immune | apoptotic and pro- | |
| | suppression | suppression | survival pathways | |
| | Neutralizes anti- | Neutralizes anti- | Lysis in Shields in | |
| | apoptotic and pro- | apoptotic and pro- | vivo Model | |
| | survival pathways | survival pathways | | |
| | Lysis in Shields | | | |
| | invivo Melanoma | | | |
| | Model | | | |
| IgG1 ADCC/CDC | Stop chemotaxis | Stop chemotaxis | Stop chemotaxis | Stop chemotaxis |
| Antibody | Lysis | Lysis | Lysis | Lysis |
| Drug Conjugated | Lysis | Lysis | Lysis | Lysis |
| Antibody | | | | |

### Example 14

### The Human Natural Ligands of CCR7: CCL19 and CCL21

To determine whether the CCR7 is expressed in its native configuration, we performed a series of experiments in which we measured the binging of natural ligands of CCR7 to cells expressing CCR7 produced by our constructs. The natural ligands of CCR7, CCL19 and CCL21 are commercially available. The mouse CCL19-Fe fusion ligand with human Fc fragment (eBioscience, catalog number 14-1972) and the human CCL-21-stalk-His6 ligand (R&D Systems, catalog number 966-6C/CF) were employed. The commercial CCL-19-Fc binds to both native mouse CCR7 according to the manufacturer's data; and human CCR7 on the cell surface, as described by Stefan Krautwald, Ekkehard Ziegler, Reinhold Förster, Lars Ohl, Kerstin Amann, and Ulrich Kunzendorf, in: Ectopic expression of CCL19 impairs alloimmune response in mice. Immunology. 2004 June; 112(2): 301-309.

Binding of the ligand to CCR7 expressing cells was demonstrated by fluorescence activated cell sorting (FACS) obtained using a Guava FACS instrument. CCL19-Fc binding to cells was detected using PE-conjugated Mouse Anti-Human Fc Monoclonal Antibody (1/50 diluted, eBioscience; catalog number 12-4998-82). Under the conditions of the experiment, if no Mouse Anti-Human Fc antibody bound to the cell, the ligand CCL19-Fc was not bound to the cell. Conversely, detection of Mouse Anti-Human Fc antibody indicated the presence of CCL19 to the cells.

To carry out these experiments, we produced cells expressing CCR7 using our expression vector. As a control, we used the same cells but not having the CCR7 expression vector.

Then, to each set of cells we added CCL-19 Fc for 30 minutes on ice. Subsequently, we added Mouse Anti-Human Fc antibody for 30 minutes on ice, after which we washed the cells to remove unbound CCL-19Fc and unbound Mouse Anti-Human Fc antibody. We fixed the cells using formalin-containing fixation buffer. We then measured fluorescence using a Guava-96 FACS device. Data is expressed as arbitrary units (PM 1 Fluorescence): CCR7 expressing cells showed much higher fluorescence (MFL=100), compared to control, parental cells that showed only marginal binding of the ligand (MFL=3.5;).

We conclude that our expression system produced CCR7 in its native configuration and therefore capable of binding the natural ligands. We further conclude that such cells expressing CCR7 can be useful for determining whether human monoclonal antibodies against CCR7 can inhibit binding of the natural ligands for CCR7.

### Example 15

### Anti-CCR Antibodies Inhibit Signaling of the Natural Ligand of CCR7

To determine if fully human antibodies against CCR7 can be useful in inhibiting CCR7 mediated disorders, we performed experiments to determine whether such antibodies can inhibit signaling by naturally occurring CCR7 ligands.

Upon binding of a natural ligand human CCL19 or CCL21 to human CCR7 on cell surface, the cell typically respond by transiently increasing intracellular concentration of calcium cations, known as Ca-flux. The Ca-flax was fluorescence measured in commercial Chem-1 cells expressing CCR7 (EMD Millipore) pre-loaded with a Ca-sensitive fluorescent dye using protocol and Calcium 5 Assay Kit of Molecular Devices (Catalog number R8185), upon addition of either CCL19 or CCL21 to final concentration 15 nM and 30 nM, respectively.

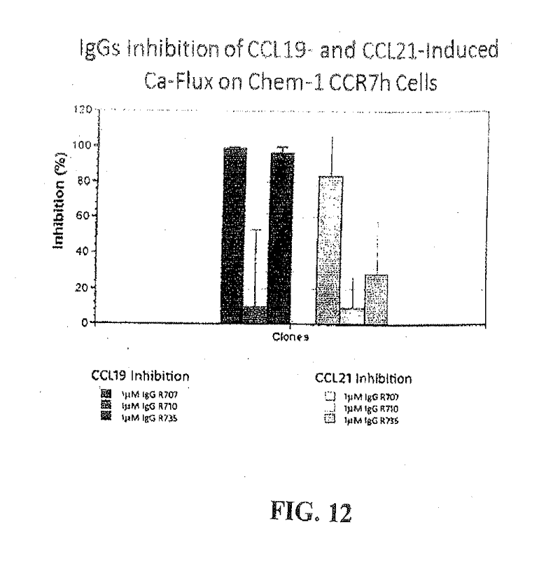

FIG. 12 depicts results of the measurement of Ca-flux in response to addition of CCL19 (three left columns), or CCL21 (three right columns) in the presence of IgG1 MSM-R707, MSM-R710, or MSM-R735 at 1 μM concentration. The data are presented as percent inhibition of a maximum Ca-flax achieved in the absence of antibodies (0% inhibition). Two antibodies, MSM-R707 and MSM-R735 displayed inhibition close to 100% for CCL19, and also inhibited Ca-signaling by CCL21, albeit to different extent—100% and 30% inhibition, respectively, whereas MSM-R710 was not capable of inhibiting signaling by either ligand.

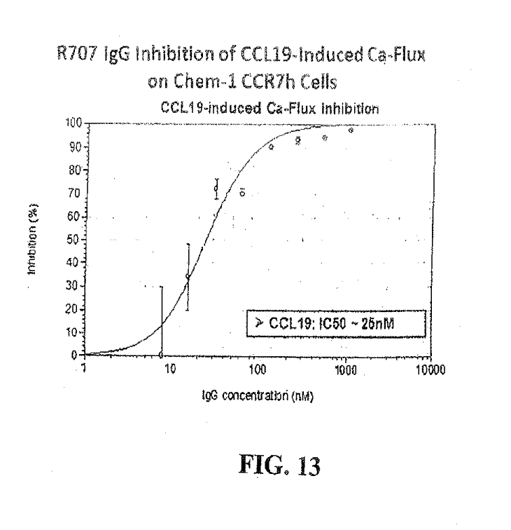

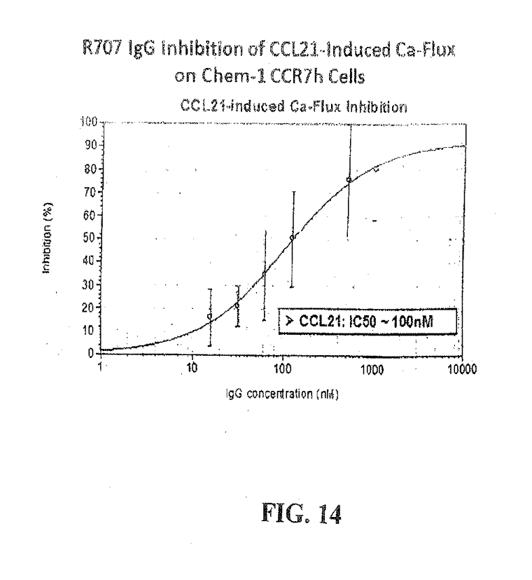

The capacity of MSM-R707 to inhibit signaling by CCL19 and CCL21 was further characterized quantitatively by measuring Ca-flux at varying concentrations of the antibody. Such a dependence is shown in FIG. 13 and FIG. 14 for CCL19 and CCL21, with 50% inhibition, i.e., IC₅₀ of 25 nM and 100 nM, respectively

We conclude from these studies that these two antibodies, IgG1 MSM-R707 and MSM-R735 inhibited signaling by natural ligands for CCR7. Without being bound by any particular theory of operation, we believe that the antibodies of this invention bound to natively configured CCR7 in such a way as to at least partially cover the binding site for the CCR7 ligand, therefore decreasing binding of the ligand for the binding site of CCR7. Regardless of the theory, we conclude that fully human antibodies of this invention can be used to decrease the effects of over-stimulation of CCR7 in a variety of conditions and disorders, including cancers.

All references cited herein are incorporated fully by reference, as if separately so incorporated.

### Example 16

### Derivatives of Anti-CCR7 Antibodies I

Other embodiments of this invention include anti-CCR7 antibodies obtained by changing one or several amino acids in CDRs by means of site directed mutagenesis. Those skillful in the art are well familiar with and often implement such an approach for generating a pool of derivative antibodies in anticipation that among so generated antibodies can be antibodies that are more suitable from the point of view of their manufacturability, storage and general stability, binding characteristics, etc. An example pool of such derivative antibodies for MSM-R707 antibody was generated with the aim of removal of two Met residues in its heavy chain CDR3, as Met residues in CDRs generally are considered undesirable from an antibody drug chemical stability perspective.

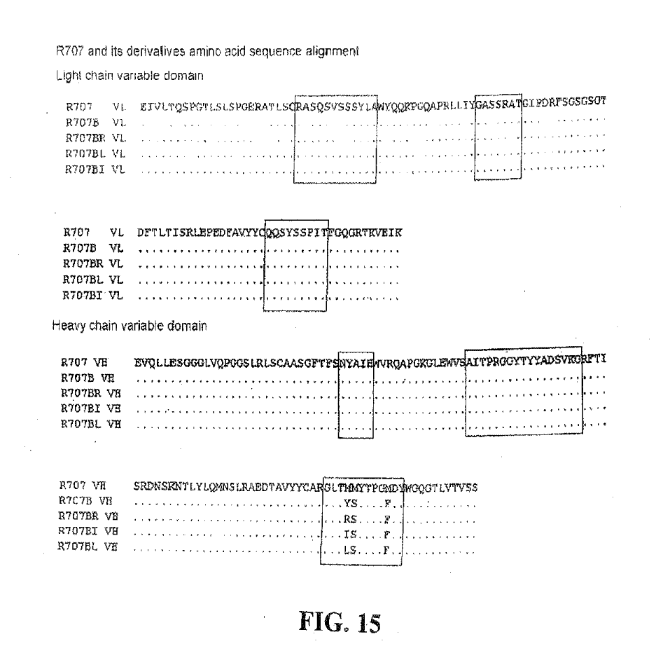

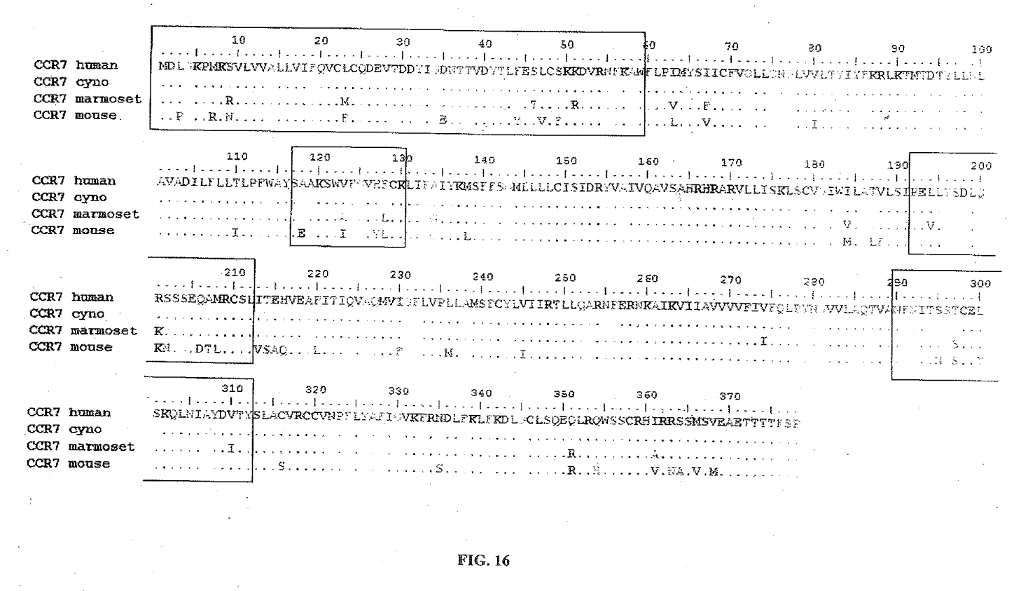

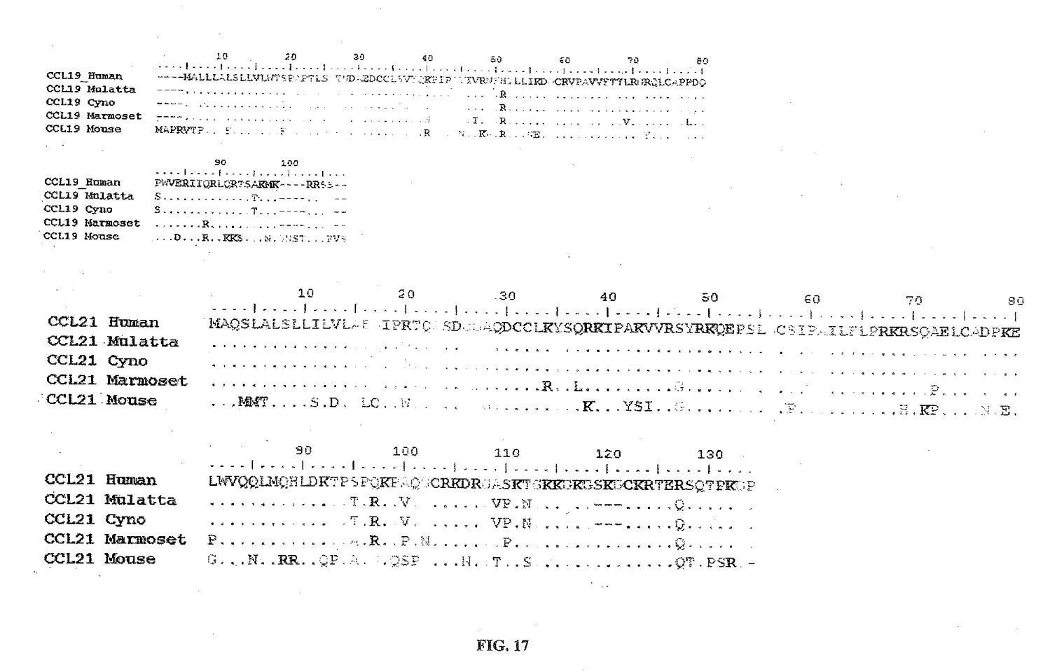

In FIG. 15 the amino acid sequence alignment for original MSM-R707 and its derivatives of this invention is provided. As was shown above, the derivatives of this invention retain or outperform certain binding characteristics. Other amino acids can be changed this way: So obtained antibodies for the CCR7 target generated, purified, and characterized to obtain derivative antibodies with improved properties. In general, derivative antibodies for the CCR7 target can have more than 80% homology as defined using either BLOSUM62 or PAM250 similarity matrix in HCDR3 alone or LCDR3+HCDR1+HCDR2 cumulatively as compared with an existing anti-CCR7 antibody of this invention. These derivative antibodies are also an embodiment of the invention.

Another approach to generating antibodies for the same target of heightened properties from an original antibody is changing amino acids similar to that known for naturally occurring somatic mutations in other than CDRs sequence regions. A possible drawback of such changes can be enhanced immunogenicity of the antibodies, which can be analyzed by a combination of analytical tools and experimentally (such services are commercially available) and potentially immunogenic antibodies discarded. This approach is known to those skillful in the art as germlining and derivatives so produced also represent an embodiment of this invention.

### Example 17

### Staining of PBMC and Splenocytes Using Monoclonal Antibodies

Peripheral Blood Mononuclear Cells (PBMC) and splenocytes were stained using the following procedure. PBMC and splenocytes (10,000 cells per well) were incubated with 100 nM of biotinylated IgG in FACS buffer (20 μL total volume per well) for 40 minutes at 4° C. The cells were then washed twice and stained for 20 minutes with either:

1. for human PBMC and Cyno PBMC, a mixture of anti-human CD4-PerCP conjugate (BioLegend #317432) and Streptavidin-PE conjugate (R&D Systems #F0040) both diluted 1:50 in FACS (10 μL per well); or

2. for mouse splenocytes, a mixture of anti-mouse CD4-PE-Cy5 conjugate (eBioscience #15-00041) and Streptavidin-PE conjugate (R&D Systems #F0040) both diluted 1:50 in FACS (10 μL per well. Then, the cells were washed twice and fixed in FIX (75 μL per well). Cells mean fluorescence intensity (MFI) was measured using a Guava PCA 96 at 485 V on a 580 nm channel and 490 V on 675 nm channel (measurements were made in 2 repeats).

The amount of IgG stained cells in CD4-positive pool was calculated according to the following formula: S1=(x1/T1)=100%, where x1 is the number of CCR7-positive/CD4-positive cells; and T1 is the total number of CD4-positive cells.

The amount of IgG stained cells in CD4-negative pool was calculated according to the following formula: S2=(x2/T2)×100%, where x2 is the number of CCR7-negative/CD4-negative cells; and T2 is the total number of CD4-positive cells. Data is expressed as the percent (%) IgG stained cells in the pool.

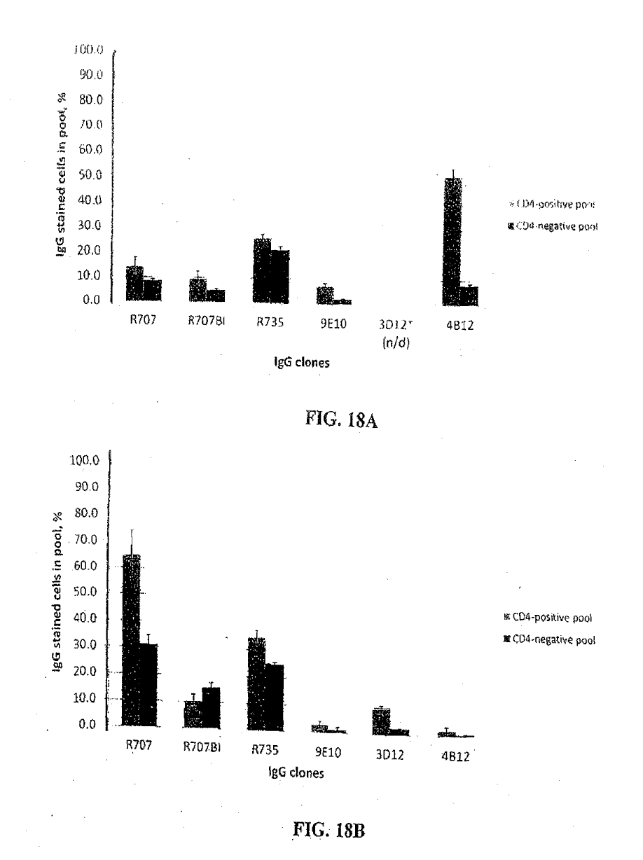

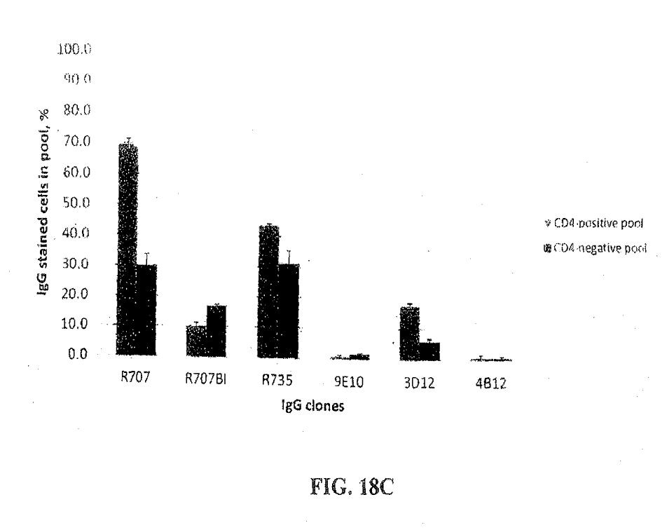

FIG. 18A depicts a graph of quantification of mouse splenocyte staining by CCR7 antibodies. R707, R707B, R735 of this invention stained more CD4-positive cells (gray bars of each pair) than CD4-negative cells (black bars of each pair). 9E10 (negative control), and 3D12 (human control) antibodies showed little staining, and 4B12 (mouse specific antibody) showed more staining of CD4-positive mouse cells than CD4-negative mouse cells.

We conclude that anti CCR7 antibodies of this invention bind mouse splenocytes, but with relatively lower affinity compared to Cyno and human CCR7.

FIG. 18B depicts results of a similar study in Cyno PBMCs. In this case, the amount of IgG binding is substantially greater than the binding to mouse splenocytes. In contrast, there was little staining by 9E10, 3D12, or 4B12 antibodies. Because the sequence of CCR7 of Cyno and humans is very similar, these results indicated that anti-CCR7 antibodies of this invention to Cyno cells is highly predictive of binding to human cells expressing CCR7.

FIG. 18C depicts results of a similar study in human PBMCs. As with the Cyno PBMCs, the anti-CCR7 antibodies of this invention bind human PBMCs with high affinity, whereas the mouse antibodies 9E10 and 4B12 did not. Only the 3D12 antibodies demonstrated binding to human CCR7-expressing cells. These studies demonstrate specificity of human CCR7 antibodies of this invention toward Cyno and human CCR7-expressing cells, whereas prior art antibodies against mouse CCR7 are only weakly effective. Thus, the antibodies of this invention can be useful tools to bind to and treat human disorders characterized by over-expression of CCR7.

### Example 18

### Binding of Anti-CCR7 Mouse Antibodies to Human Leukemia Cells

To determine whether anti-CCR7 antibodies of this invention can bind to human leukemia cells, we carried out s series of studies in which we compared mouse anti-CCR7 antibodies and mouse anti-CD22 antibodies to a human leukemia cell line, CLL-ATT (B-Cell Chronic Lymphocytic Leukemia; “B-CLL”) and JVM-13 cells (B-Cell Prolymphocytic Leukemia, “B-PLL”).

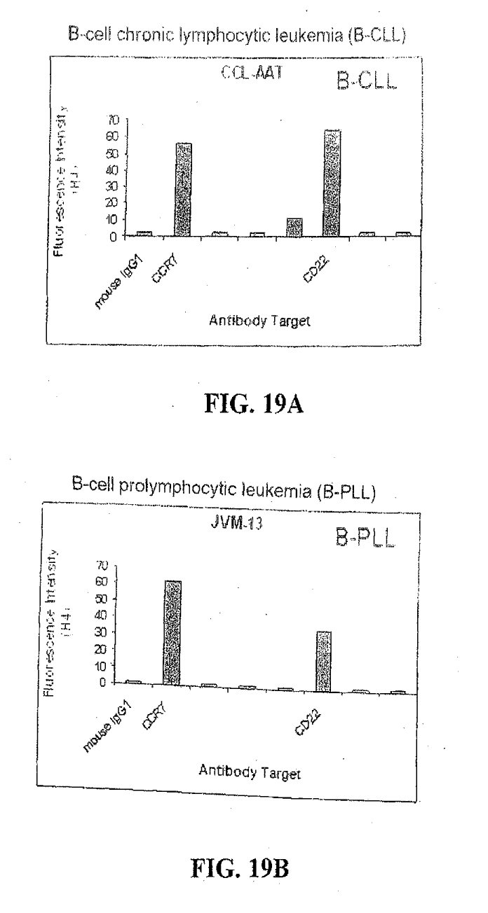

FIG. 19A depicts a graph showing that mouse antibodies against CCR7 and CD22 bind to B-CLL cells, but mouse IgG1 did not.

FIG. 19B depicts a graph showing that antibodies against CCR7 and CD22 bound to B-PLL cells, but mouse IgG1 did not.

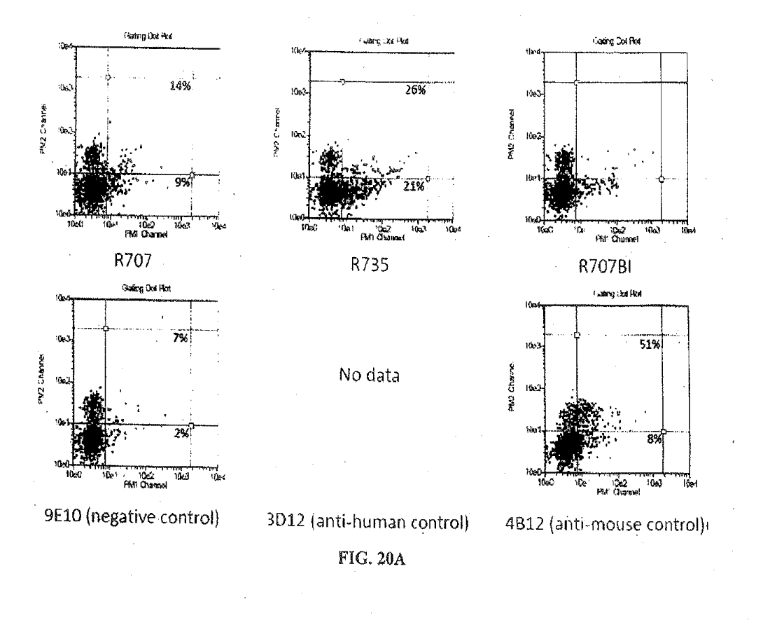

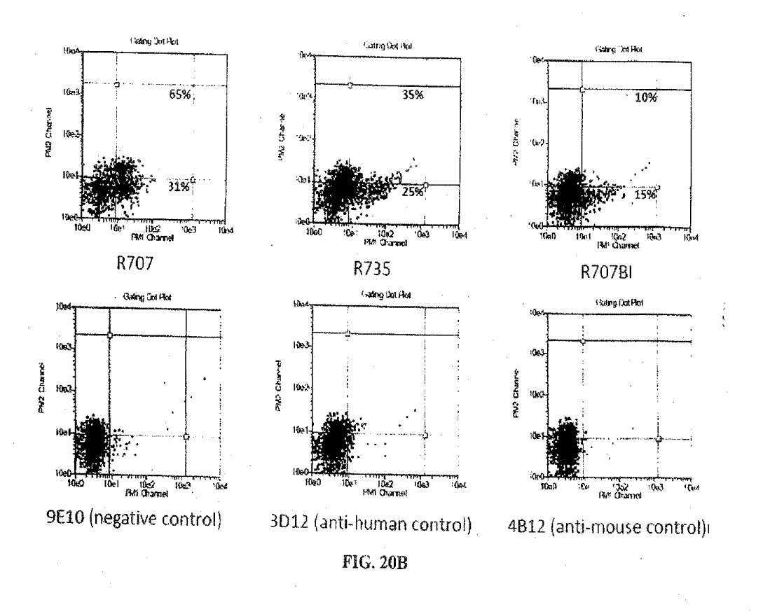

FIG. 20A depicts cell sorter plots for mouse splenocytes stained with anti-CCR7 antibodies of this invention. The upper panels show results for R707 (top left panel), R735 (top middle panel) and R707B1 (top right panel). In each case, there was a population of cells with greater than the gated number of cells, shown to the right of the vertical line in each plot. In contrast, the bottom panels showed little staining by 9E10 (negative control; bottom left panel). We did observe binding of 4B12 (mouse anti-CCR7 antibody; bottom right panel) to mouse splenocytes. No data was obtained for 3D12 (human control). We conclude that anti-CCR7 antibodies of this invention can bind to mouse splenocytes, and that studies of mouse splenocytes can be reasonably predictive of effects in human beings.

### Example 19

### Binding of CCR7 Antibodies of this Invention to Cyno PBMCs

To study the binding of anti-CCR7 antibodies of this invention to Cyno PBMCs, we carried out s series of studies using isolated PBMCs from Cynomologous monkeys.

FIG. 20B depicts results of studies similar to those carried out using methods of Example 18. In the top panels, antibodies of this invention, R707 (top left panel), R735 (top middle panel), and R707B1 (top right panel) showed significant numbers of cells showing binding to PBMCs. Positively staining cells are shown to the right of the vertical line in each panel.

In contrast, FIG. 20B shows little staining of calls eith 9E10 (negative control; bottom left panel), 3D12 (anti-human control; bottom middle panel) or 4B12 (anti-mouse control; bottom right panel).

### Example 20

### Binding of CCR7 Antibodies of this Invention to Human PBMCs

In a similar study to that shown in Example 19, we studied the binding of anti-CCR7 antibodies of this invention to human PBMCs.

FIG. 20C depicts cell sorter graphs of the results. As with FIG. 20B, R707 (top left panel) demonstrated substantial binding of R707 to human PBMCs. Similarly, R735 demonstrated substantial binding (top middle panel), and R707B1 showed substantial binding to human PBMCs (top right panel). In contrast, 9E10 (negative control; bottom left panel), 3D12 (anti-human control; bottom middle panel) and 4B12 (anti-mouse control; bottom right panel) showed little or no binding to human PBMCs.

We conclude from Examples 18-20 that human anti-CCR7 antibodies of this invention can bind strongly and with specificity to human and Cyno cells, meaning that studies using Cyno cells is highly predictive of effects observed for human cells. We further conclude that although the binding of antibodies of this invention bind to mouse splenocytes less well than to either Cyno or human cells, nonetheless, studies using mouse cells is reasonably predictive of effects in human beings.

### Example 21

### Differential Binding of CCR7 Antibodies of this Invention to Different Molecular Subsets of CCR7

In another series of experiments, we studied binding of CCR7 antibodies of this invention to different molecular targets on CCR7. To do this, we plotted the MFI (vertical axis) of Chinese Hamster Ovary (CHO) cells expressing human CCR7 against the concentration of IgG (in nM).

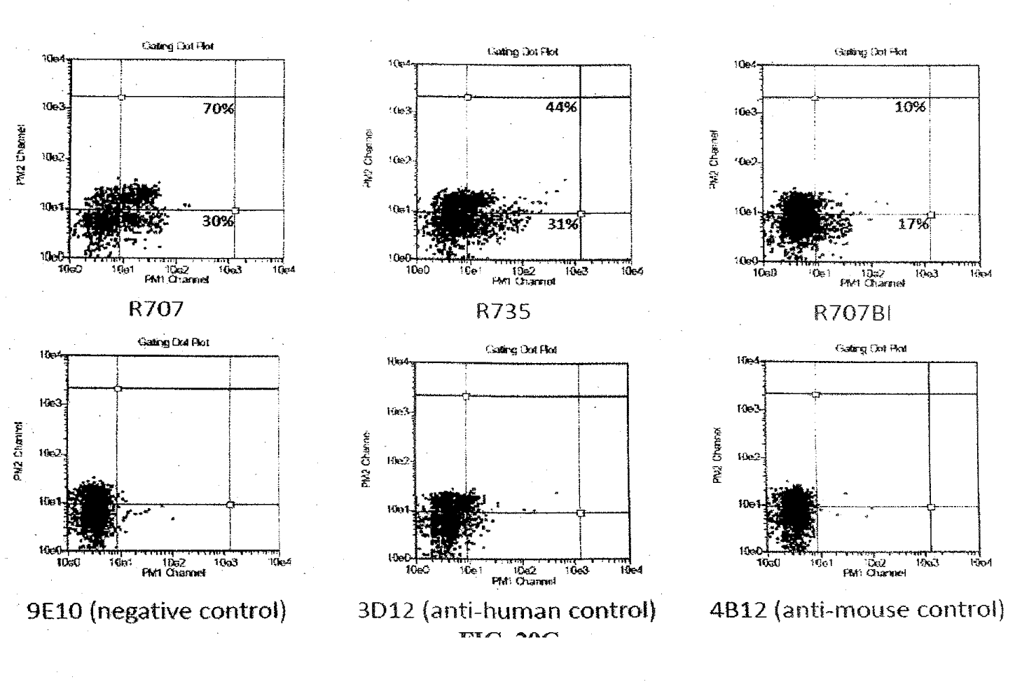

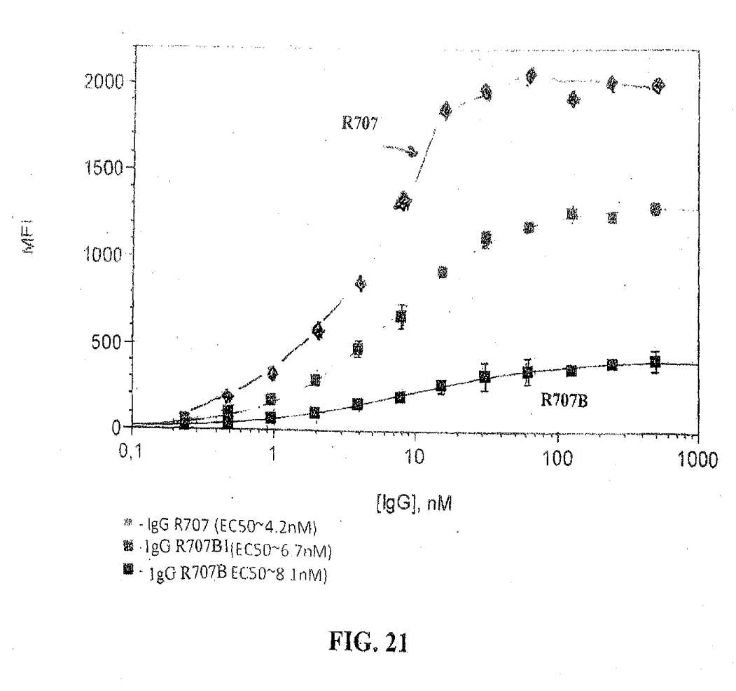

FIG. 21 depicts the results of this study. R707 (top graph) shows the highest maximal binding to CHO cells, R707B1 shows an intermediate maximal binding, and R707B shows the lowest maximal binding. However, the affinity of binding for each of these antibodies of this invention are in the nM range, with R707, R707B1, and R707B having EC₅₀s of 4.2 nM, 6.7 nM, and 8.1 nM, respectively. In striking contrast to these results, we detected no binding of any of the antibodies of this invention, IgG 150503 (R&D Systems), IgG 3D12 (eBioscience) or IgG 4B12 (R&D Systems) to parental CHO cells (not expressing human CCR7). This study demonstrated that anti-CCR7 antibodies of this invention bind to different molecular subsets of CCR7, and therefore, different anti-CCR7 antibodies can be useful to treat disorders in which one or other molecular subsets of CCR7 may be inaccessible to antibody therapy.

### Example 22

### Binding of Anti CCR7 Antibodies of this Invention to CCR7-Expressing CHO Cells

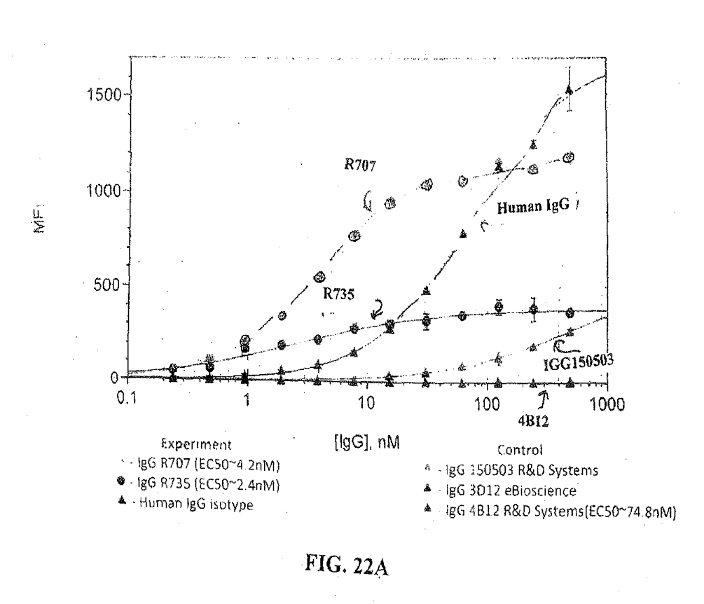

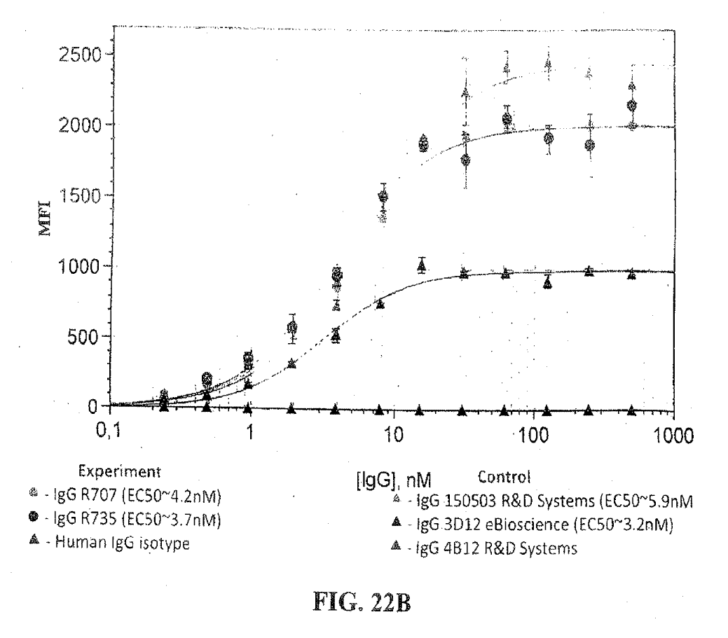

In a study similar to that in Example 21, we compared binging of anti-CCR7 antibodies of this invention to CHO cells expressing mouse CCR7. As depicted in FIG. 22A, binding of CCR7 antibodies of this invention (R707, and R735) to mouse cells expressing CCR7 showed EC₅₀s of 4.2 nM and 2.4 nM, respectively. In contrast, other antibodies (controls) either bound weakly or not at all.

FIG. 22B shows results of binding of antibodies of this invention (R707, and R735) to human CCR7-expressing CHO cells. Both R707 and R735 bound to these cells with high affinity (EC₅₀s of 4.2 nM and 3.7 nM, respectively. Table 13 below depicts a summary of the binding experiments described above.

TABLE 13
CCR7 Monoclonal Antibody Binding Properties

| | Human | Cyno | Mouse | | | |
|---|---|---|---|---|---|---|
| | CCR7 | CCR7 | CCR7 | Human | Cyno | Mouse |
| IgG | EC₅₀ (nM) | EC₅₀ (nM) | EC₅₀ (nM) | PBMC | PBMC | PBMC |
| R707 | 4.7 | * | 4.3 | Yes | Yes | Yes |
| R735 | 4.0 | * | 1.0 | Yes | Yes | Yes |
| 150503 | 6.9 | * | No | No data | No data | No data |
| 3D12 | 3.4 | * | No | Yes | Yes | No data |
| 4B12 | No | No data | 73.5 | No | No | Yes |

*: Because the sequences of Cyno and human CCR7 area nearly identical, the EC₅₀ will be very similar to that observed for human CCR7.

### Example 23

### Binding of Human CCR7 Antibodies of this Invention to Cancer Cells

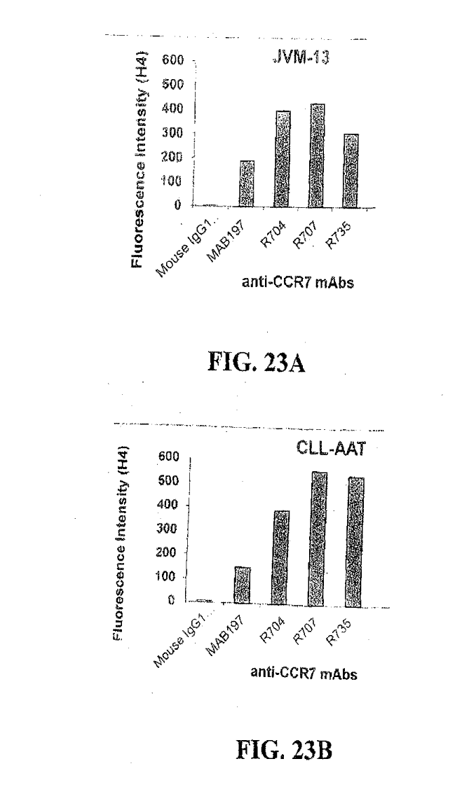

Having shown that the CCR7 antibodies of this invention bind to cells that express CCR7, we then carried out studies to determine if the fully human anti-CCR7 antibodies bind to cancer cells. To do this, we selected 3 cell lines, JVM-13, CCL-AAT, and BHK/CCR7. In general the methods used were similar to those used in prior examples for CCR7-expressing cells. Results of these studies is shown in FIGS. 23A-B depict graphs of anti-CCR7 antibodies of this invention (horizontal axis) vs. intensity of florescence (vertical axis). FIG. 23A shows that R704, R707, and R735 of this invention bind strongly to JVM-13 cells. FIG. 23B shows that those antibodies also bind strongly to CLL-AAT cells Monoclonal antibody MAB197 binds relatively weakly to both JVM-13 and CLL-AAT cells, and mouse IgG1 binding is absent in JVM-13 cells and is barely detectable in CLL-AAT cells.

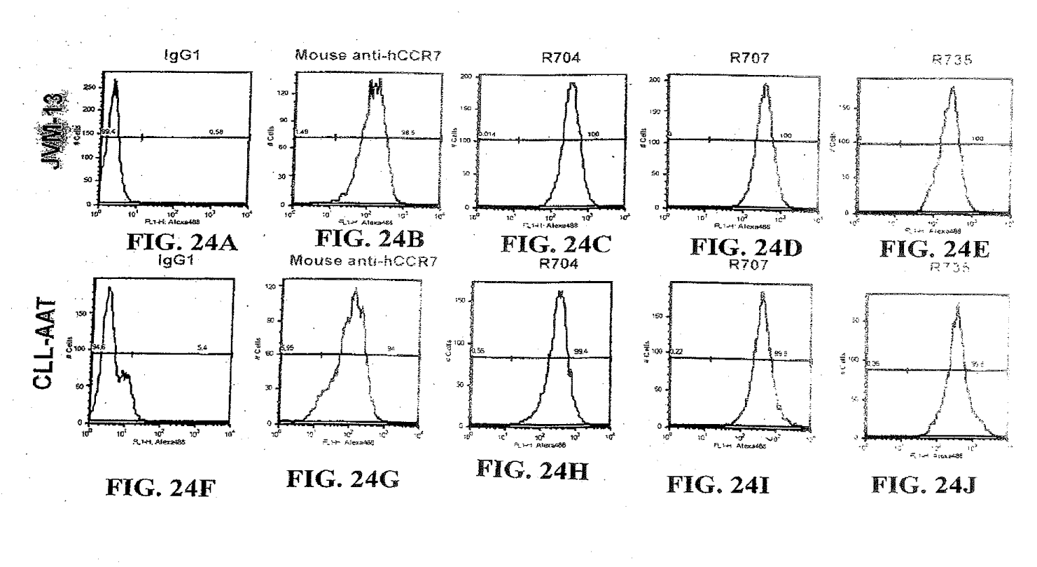

FIGS. 24A-J depict cell sorter plots of JVM-13 cells (top row, FIGS. 24A-E), and CLL-AAT cells (bottom row; FIGS. 24F-J). Each plot is of florescence intensity (horizontal axis) vs. the number of cells detected (vertical axis). FIGS. 24A and F show low florescence intensity after exposure of the cells to IgG1. In contrast, after exposure to mouse anti-human CCR7 antibodies (FIGS. 24B and 24G), the intensity of fluorescence increased substantially. Similarly, fully human antibodies against human CCR7 (FIGS. 24C-E and FIGS. 24H-J) showed substantial florescence.

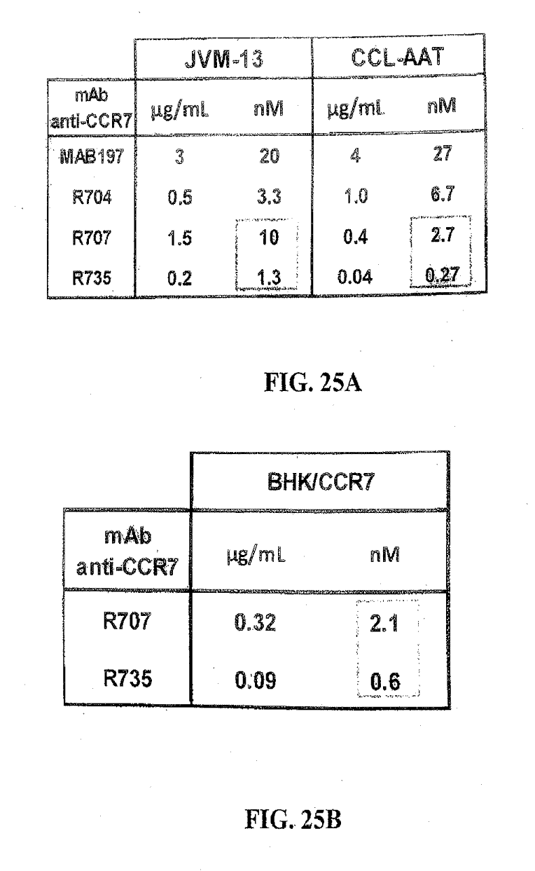

FIGS. 25A-B depict summary data of binding of antibodies of this invention to JVM-13 cells (FIG. 25A), CCL-AAT cells (FIG. 25A) and BHK/CCR7 cells (FIG. 25B).

These results indicate that human cancer cell lines bind fully human antibodies against CCR7, and can therefore be effective agents in treating disorders characterized by over expression or over-stimulation of CCR7.

### Example 24

### Cytotoxicity Assays Using Non-Human Antibodies

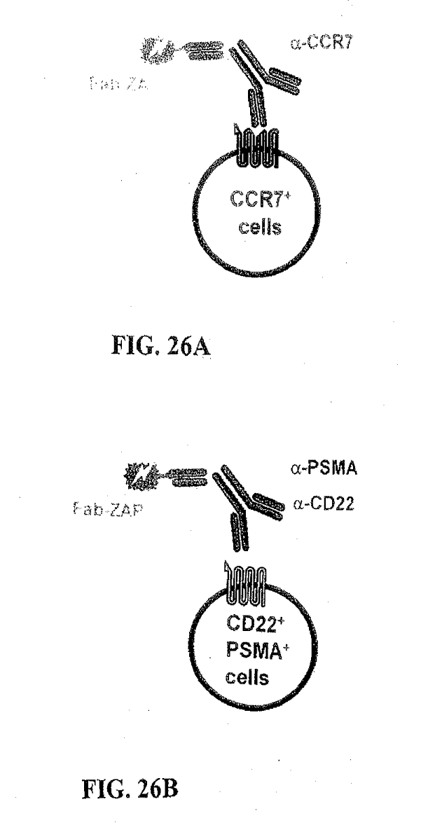

To determine whether mouse human anti-CCR7 monoclonal antibodies can be cytotoxic, we carried out a series of studies using a commercially available reporter antibody fragment conjugated to a potent bacterial toxin, Saponin (Fab-ZAP™). To carry out the experiments, we compared effects of murine anti-CCR7 antibodies of this invention coupled to Fab-ZAP in killing CCR7+ cells (FIG. 26A). For comparison, we also studied two prior art antibodies (anti-prostate specific monoclonal antibody (PSMA) and anti-CD22, coupled to Fab-ZAP for their abilities to kill CD22+ and PSMA+ human cells, respectively (FIG. 26B).

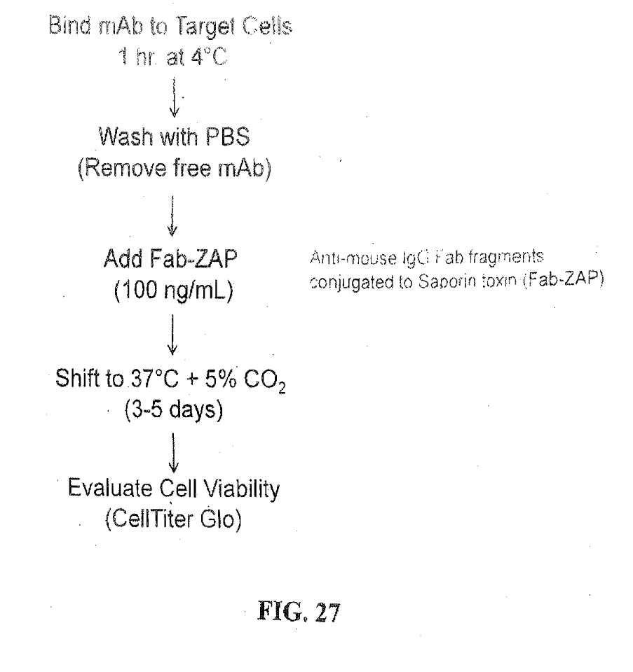

The protocol for these experiments is depicted in FIG. 27.

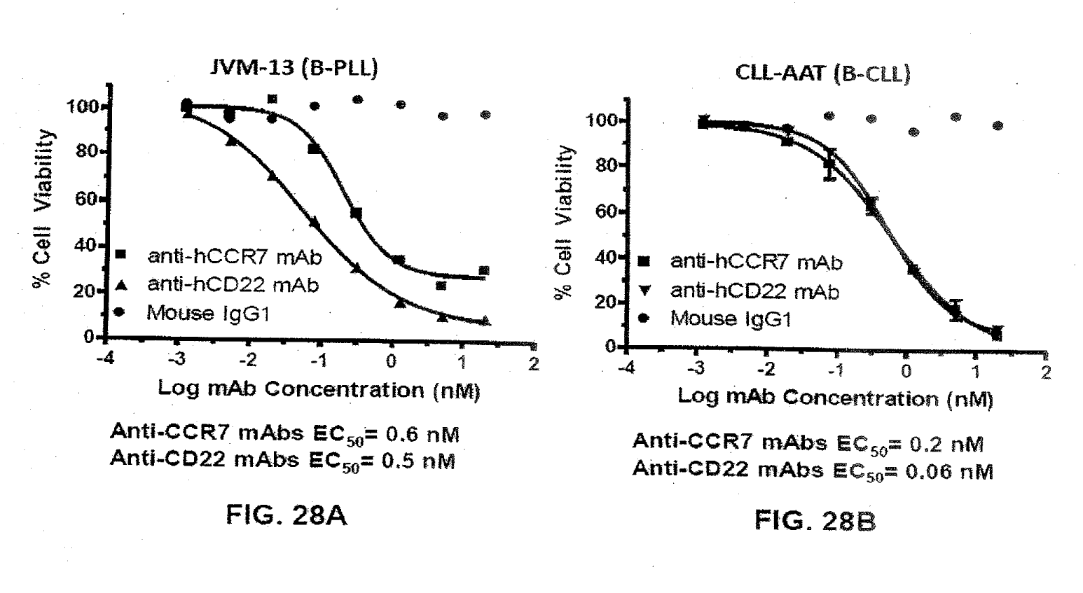

FIGS. 28A and 28B depict results of these experiments. FIGS. 28A and 28B depict graphs of the log of the concentrations of mouse antibodies (in nM; horizontal axis) vs. the % cell viability (vertical axis) in JVM-13 cells (FIG. 28A) and in CLL-AAT cells (FIG. 28B). FIG. 28A shows that in the JVM-13 cells, both anti-CCR7 human antibodies and anti-human CD22 antibodies result is a concentration-dependent cell killing, with EC₅₀s of 0.6 nM and 0.5 nM, respectively. FIG. 28B shows the results of the studies of CLL-AAT cells, in which anti-human CCR7 and anti-CD22 monoclonal antibodies have EC₅₀s of 0.2 nM and 0.06 nM, respectively.

### Example 25

### Cytotoxicity Assays with Human Monoclonal Antibodies

Having proved the concept that murine antibodies against CCR7 and CD22 can effectively kill human cell lines expressing CCR7, we then carried out a series of experiments using fully human antibodies of this invention. FIG. 29 depicts the methods used to carry out these studies.

FIG. 30 depicts a graph of log of the antibody concentration (in nM; horizontal axis) vs. % cell viability (vertical axis) results of these studies using JVM-13 cells. For R704, R707 and R735 of this invention, we observed a concentration-dependent decrease in cell viability, with a threshold of about 0.5 nM, and IC₅₀s of about 1.2 nM. Similar results were observed for the murine anti-human CCR7 monoclonal antibody. In contrast, human IgG1 had no effect.

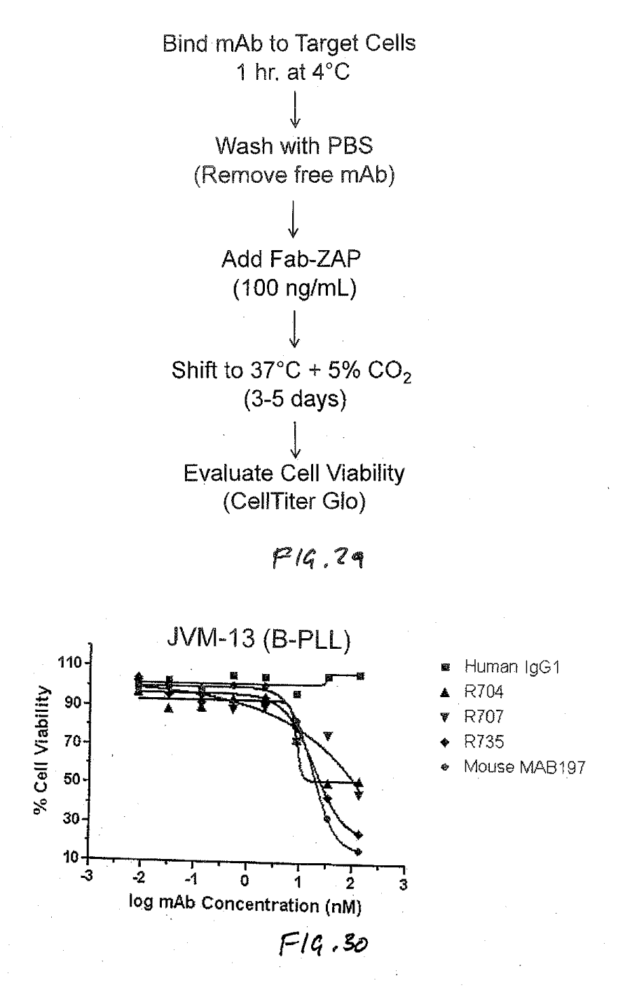

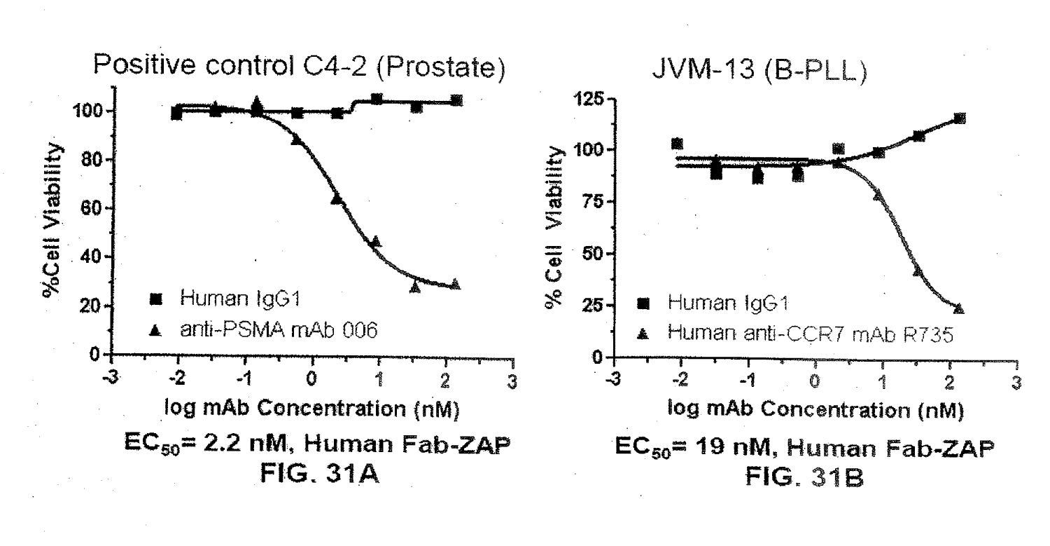

FIGS. 31A and 31B depict results of studies of C4-2 cells (prostate cancer cell line) and JVM-13 cells, in response to anti-PSMA mAbs (FIG. 31A) or human anti-CCRY mAb R735 (FIG. 31B), respectively. FIG. 31A shows that anti-PSMA mAbs are highly effective in killing C4-2 cells, with an EC₅₀ of 2.2 nM. FIG. 31B shows that fully human anti-CCR7 monoclonal antibody R735 is highly effective in killing JVM-13 cells, with an EC₅₀ of 19 nM. In contrast with these effects of monoclonal antibodies, human IgG1 did not show any cell killing at the concentrations tested.

We conclude that fully human anti-CCR7 antibodies of this invention can be effective in delivering a cytotoxic molecule to cells, and are capable of effectively killing the cells. These effects were specific to the antibodies used, and we therefore conclude that the antibodies of this invention can be useful in targeting cancer cells. Because the study designs and materials are considered to be reasonably predictive of therapeutic effects in human beings, the fully human antibodies of this invention can be therapeutically useful to treat disorders involving over-expression or over-stimulation of CCR7.

### Example 26

### Methods for Detecting and Quantifying Receptor Internalization

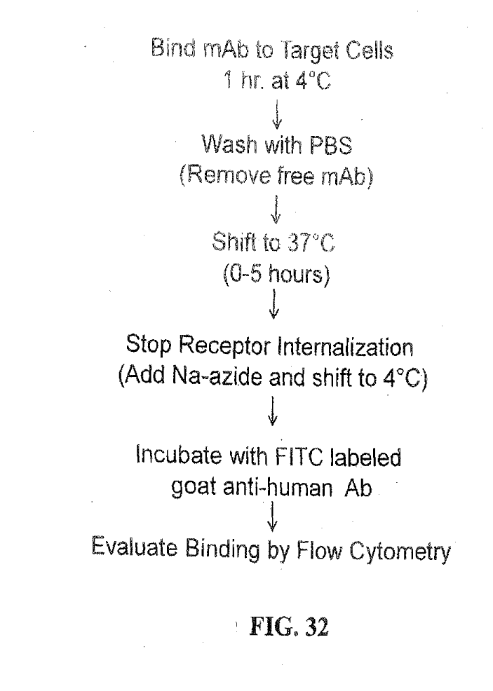

The studies described in Example 25 demonstrated that antibodies of this invention can be useful in targeting CCR7 in human cell lines. As a potential alternative mechanism of action, we also determined whether the antibodies of this invention could provoke CCR7 internalization. FIG. 32 depicts a flow chart for the method used in these experiments. In this assay, we measured the maximum binding of anti-CCR7 antibodies to the cells, which is a measure of the presence of cell-surface CCR7 bound to anti-CCR7 antibodies. A decrease in maximal binding therefore reflects internalization of the antibody-CCR7 complex.

FIG. 33 depicts a graph of time at 37° C. (horizontal axis) versus percent of maximum binding of maximal binding of the antibody to CCL-AAT cells as detected by flow cytometry. As can be clearly observed, R707 of this invention produces a rapid and time-dependent loss of maximal binding. R735 also shows a reduction in maximal binding. We conclude that R707 and R735 can be effective in internalizing CCR7 on CCR7 producing cells.

FIG. 34 depicts a graph of time at 37° C. vs. the percent of maximum binding, to C4-2 prostate cancer cells. As can be readily results for C4-2 prostate cancer cells and the effects of mouse anti-PSMA or human anti-PSMA antibodies. FIG. 34 shows that both murine anti-PSMA and anti-human PSMA were effective in decreasing the maximal binding of the respective antibodies to the cells' surface.

We therefore conclude that the assay methods used and results obtained for studies of internalization of CCR7 described in FIG. 33 are validated. We also conclude that internalization of CCR7 is a potent mechanism of anti-CCR7 antibodies of this invention. In addition to being effective in targeting toxins to cells, internalization of CCR7 is another mechanism by which anti-CCR7 antibodies of this invention can be therapeutically useful.

### Example 27

### Effects of Monoclonal Antibodies of this Invention on Chemokine-Induced Calcium Flux I

Having shown that antibodies of this invention bind to CCR7 on intact human cells, can effectively be used to target toxins to cells to kill them, and can internalize CCR7 receptors, we then carried out a series of studies to determine whether antibodies of this invention can affect the normal cell signaling pathways of CCR7-expressing cells.

To do this, we used the following experimental protocol. Chem-1 CCR7 human cells (Millipore), were starved in serum-free media (CHO-FreeStyle™) for 5 hours at 37° C. in 5% CO₂ in air. The cells were then loaded with dye (Calcium 5 kit) with different concentrations of IgG 150503 (tittering from 0.5 μM with dilutions factor of 2.5 for 30 minutes at 37° C. and 5% CO₂ in air. CCL19 (R&D Systems) in TBS with 1 mM CaCl₂ was added to dye-loaded cells to reach concentrations of 15 nM. Inhibition of calcium flux was calculated according to the follow formula:

Percent Inhibition=(1−[I]/[C])×100%,

where I is the mean peak value (n=4) in inhibited samples, and C is the mean peak value (n=18) in control samples.

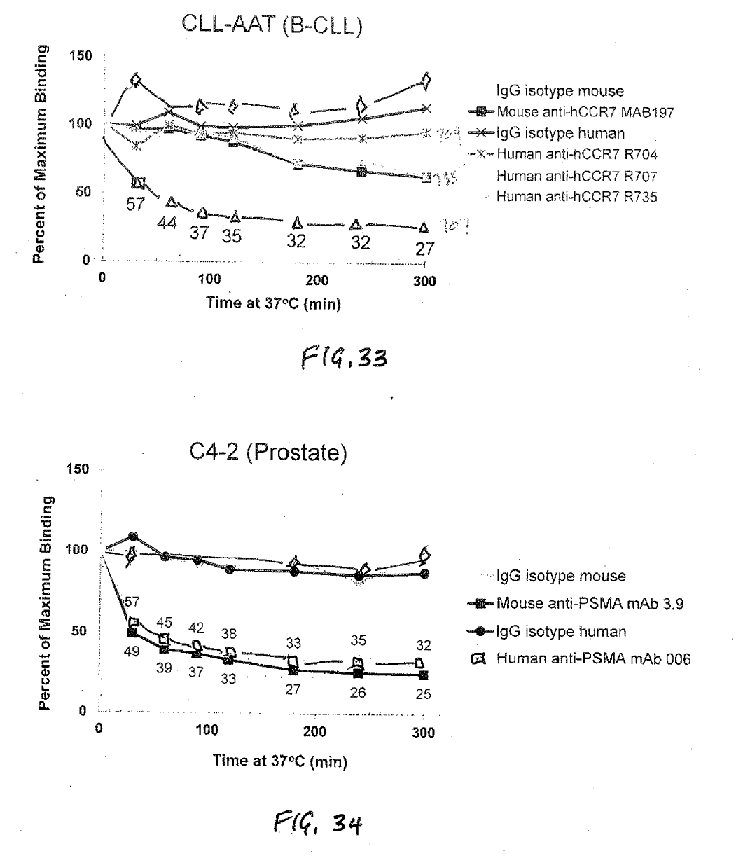

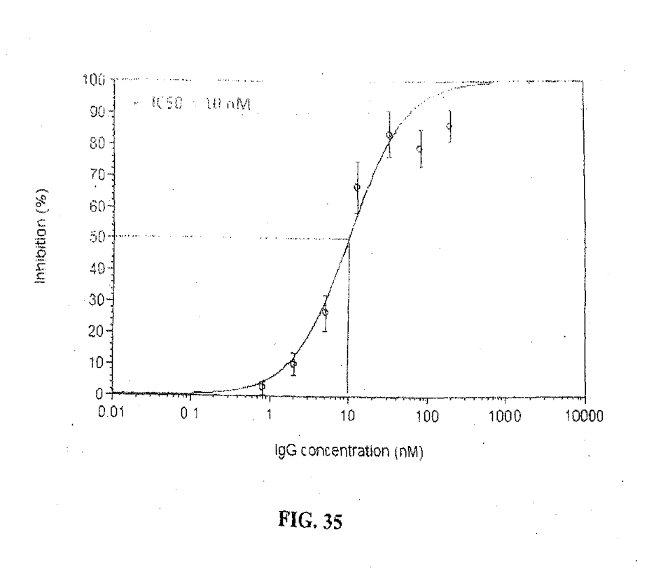

FIG. 35 depicts a graph of IgG concentration (in nM; horizontal axis) and % inhibition of calcium flux (vertical axis). Ac can be seen from FIG. 35, IgG 150503 reduced CCL19-induced calcium flux in a concentration-dependent fashion, with an IC₅₀ of 10 nM. We conclude from FIG. 35, that the methods used are suitable for studies of effects of anti-CCR7 antibodies of this invention.

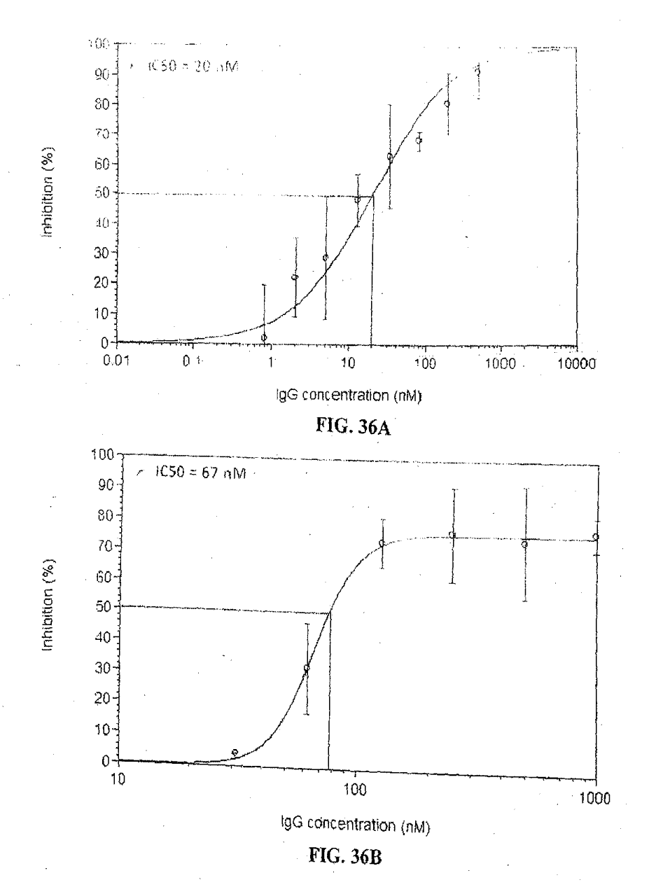

FIG. 36A depicts a study, similar to that shown in FIG. 35, but where R707 was used. As shown in FIG. 35, the fully human anti-human CCR7 monoclonal antibody R707 inhibited CCL 19-induced calcium flux in a concentration-dependent fashion, with as threshold of about 30 nM, an IC₅₀ of about 20 nM and a maximal effect at about 120 nM.

Similar results were obtained for the fully human anti-human CCR7 antibody R735 of this invention. FIG. 36B depicts a graph of IgG concentration (in nM) versus % inhibition of calcium flux. R735 caused a concentration-dependent inhibition of calcium flux with a threshold of about 30 nM, an IC₅₀ of 67 nM, and a maximal effect at about 105 nM.

We conclude from these studies that anti-CCR7 antibodies of this invention can be used to inhibit normal cellular signaling (calcium flux) in cells that express CCR7, and therefore can be effective in inhibiting calcium-dependent effects, including chemotaxis, thereby being useful in inhibiting migration of cancer cells, and thereby decreasing metastasis.

### Example 28

### Thermal Stability of Anti-CCR7 Monoclonal Antibodies

Having demonstrated several desirable effects of fully human anti-human CCR7 monoclonal antibodies, we then carried out a series of studies to determine their suitability for use as drugs for human therapy. To do this, we studied several variables of production, including expression rate, protein integrity, temperature sensitivity and shelf life.

First, we studied IgG expression for R707 and R735 in a transient expression system from NRC. We compared this expression with that of MDX-1338 (an anti-CXCR4 human IgG from Medarex as a positive control. We found that R707 was expressed at a level of 35 mg/L, R735 was expressed at a level of 50 mg/L, and MDX-1338 was expressed at a level of 20 nag/L. We conclude that expression of R707 and R735 are substantially higher than the positive control MDX-1338.

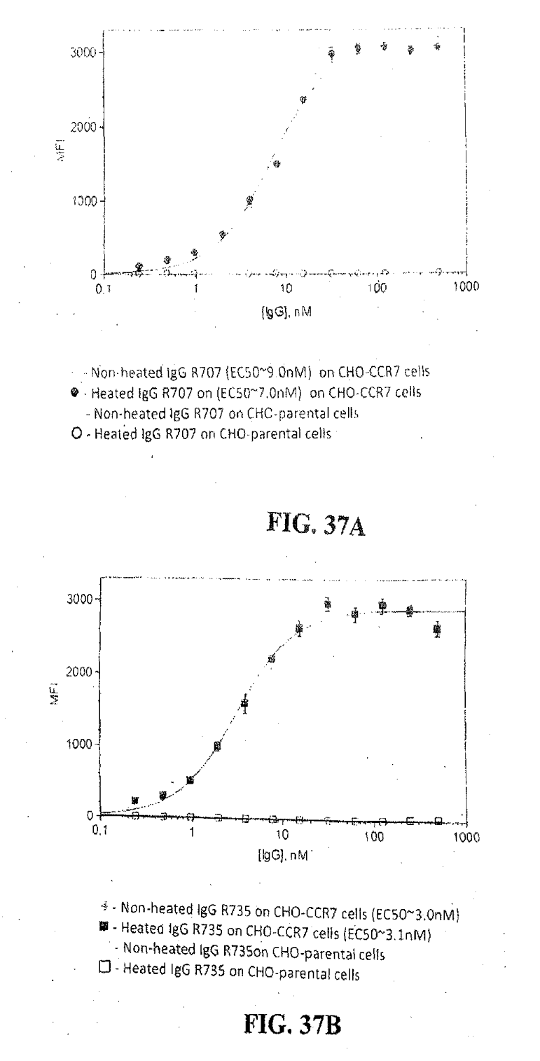

Next, we studied the thermal stability of fully human anti-human CCR7 monoclonal antibodies. To do this, we treated antibodies at high temperature (40° C. for 12 hrs) and then determined the biding of either heat-treated or untreated antibodies to CHO cells that expressed CCR7. FIGS. 37A and 37B depict graphs of antibody concentration (IgG in nM; horizontal axis) vs. mean fluorescence intensity (by cell sorter; vertical axis) as described previously for the binding studies.

FIG. 37A shows results for R707. Non-heat treated R707 (gray circles) shows binding to CCR7-expressing CHO cells. The effect was concentration-dependent, with an EC₅₀ of 9.0 nM. Heat treated R707 had a similar binding curve, with an EC₅₀ of 7.0 nM. In contrast, there was no binding of either heat-treated or non-heat treated CCR7 to CHO parental cells.

FIG. 37B shows results of R737. Both non-heat treated and heat-treated CCR7 found to CCR7-expressing CHO cells with similar affinity (EC₅₀s of 3.0 nM and 3.1 nM, respectively). As with R707, neither heat-treated nor non-heat-treated R737 bound to parental CHO cells not expressing CCR7.

We conclude from this study, that heat treatment had little adverse effect on the ability of anti-CCR7 antibodies of this invention to retain their normal binding properties to CCR7 on cells. Therefore, preparations of these antibodies can retain potency under thermal stress.

In another study of thermal stability, we determined whether heat treatment caused degradation of the IgG molecules. To do this, we exposed IgG samples of this invention to a temperature stress (40° C. for 12 hours), and then analyzed molecular size using SDS gel polyacrylamide gel electrophoresis (SDS-PAGE).

FIGS. 38A-D depict photographs of SDS gels. Lanes 0 and 7 are size control ladders. Lanes 1-2 of each photograph are for R707, and lanes 34 are for R735. Lanes 5 are for a control antibody (MDS-1338), and lanes 6 are for Rituximab. Gels were run in either non-reducing conditions (FIGS. 38A and 38B) or reducing conditions (FIGS. 38C and 38D). In the absence of heat treatment (FIG. 38A), the size of each of the antibodies in the non-reducing conditions ran with their expected molecular size (consistent with IgG). As expected, under reducing conditions (FIG. 38C) each of the antibodies was dissociated into light chains and heavy chains, each having the characteristic mobility of IgG. After heat treatment (FIG. 38B) run in non-reducing conditions, each of the antibodies migrated identically to the non-heat-treated samples, indicating that heat treatment did not degrade the antibodies. FIG. 38D shows that under reducing conditions, the mobility of each of the portions of IgG (light chains below and heavy chains above) had the same mobility as those of the non-heat-treated antibodies.

We conclude from this study that heat treatment does not degrade either the intact IgG (FIGS. 38A and 38B) or the light chains or heavy chains (FIGS. 38C and 38D). These results also confirm that the methods used to study anti-CCR7 antibodies of this invention are validated by similar results obtained for a prior art antibody (MDX-1338). We therefore conclude that heat-treatment does not adversely affect either binding nor molecular integrity of the antibodies of this invention.

### Example 29

### Anti CCR7 Antibodies are Resistant to Proteolysis

To determine whether antibodies of this invention are susceptible to degradation by proteolytic enzymes, we carried out a series of studies using the general protease, trypsin. To carry out these studies, we exposed IgG of this invention to trypsin (0.025% (Tissue Culture Grade) at 37° C. for 30 minutes. Parallel samples were not exposed to trypsin. Then, the samples were run on a 4% to 20% gradient SDS gel under reducing conditions. A control degraded IgG (V62) was run as a positive control. R735 (lanes 3 and 4) or R707 (lanes 5 and 6) were either treated with trypsin (lanes 3 and 5) or not treated with trypsin (lanes 4 and 6).

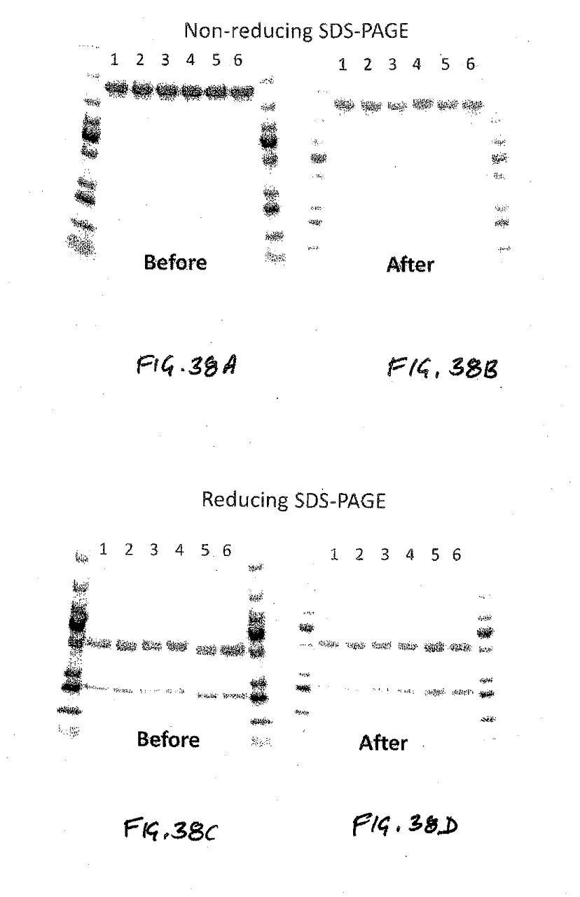

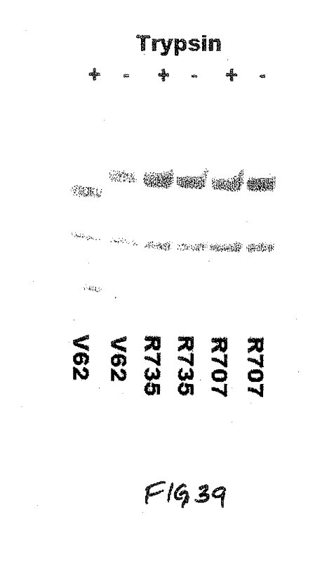

Results are shown in FIG. 39, which is a photograph of the SDS gel. As expected, when run under reducing conditions, there are two bands in the non-trypsin treated samples. The upper band represents the IgG heavy chain and the lower band represents the light chain.

In the non-trypsin treated control (V62; FIG. 39 lane 2), there were the two expected bands (heavy and light chains) begin dissociated under reducing conditions. In contrast, the trypsin-treated positive control (V62; FIG. 39 lane 1) showed three bands, the lowermost representing a digestion fragment, and the upper band running at a lower molecular size than the non-trypsin-treated sample. This, lanes 1 and 2 demonstrate proteolysis of V62.

In contrast to the proteolytic effect of trypsin on V62, there was no detectable proteolysis of either R735 or R707. In both cases, we observed the two bands (heavy and light chains) being dissociated from each other under reducing conditions.

We conclude from these studies that anti-CCR7 antibodies of this invention are resistant to proteolysis and that they therefore are stable under these conditions.

### Example 30

### Shelf Life of Anti-CCR7 Antibodies of this Invention

Having shown that the fully human anti-human CCR7 antibodies of this invention are stable under thermal stress and proteolysis, we studied the stability of these antibodies to prolonged storage. To do these studies, we stored IgG samples for 4.5 months at 4° C. in a solution of storage buffer. After storage, we determined the binding properties of the stored or un-stored antibodies to cells expressing CCR7. FIGS. 40A and 40B depicts graphs of IgG concentration (in nM; horizontal axis) vs. mean fluorescence intensity as determined by cell sorter.

FIG. 40A shows results for R707. Before and after storage, the two samples showed very similar binding curves, with EC₅₀ in October 2011 of 1.80 nM, and in February 2012 of 1.63.

FIG. 40B shows results for R737. Before and after storage, the two samples showed very similar binding curves, with EC₅₀ in October 2011 of 2.47, and in February 2012 of 2.06.

We conclude from these studies that anti-CCR7 antibodies of this invention can be stored for prolonged periods, making them suitable for use as therapeutic agents.

Based on the above-described Examples 28-30, we conclude that the fully human anti-human CCR7 antibodies of this invention are thermally stable, resistant to proteolysis, and have a suitably long shelf life to be produced, compounded, transported and stored without substantial loss of efficacy. These properties, along with the demonstrated high binding affinity, ability to effectively target and kill cells that express CCR7, to inhibit normal cell signaling by inhibiting normal responses to the endogenous CCR7 ligand (CCL19) mean that the antibodies of this invention can be useful in therapy of disorders characterized by over-expression of CCR7 or by over-stimulation of CCR7 by endogenous ligands.

### Example 31

### Inhibition of Chemokine CCL19-Induced Chemotaxis

In another study, we determined whether fully human anti-human CCR7 monoclonal antibodies of this invention affect metastasis induced by chemokine CCL19. To do this, we studied CLL-AAT cells starved in serum-free cultivation media (IMDM with 50 μM of β-ME) overnight at a temperature of 37° C. in 5% CO₂ in air, and then were incubated with 500 nM IgGs in serum-free cultivation media 20 min, 37° C., 5% CO₂. Control samples were incubated with media only.

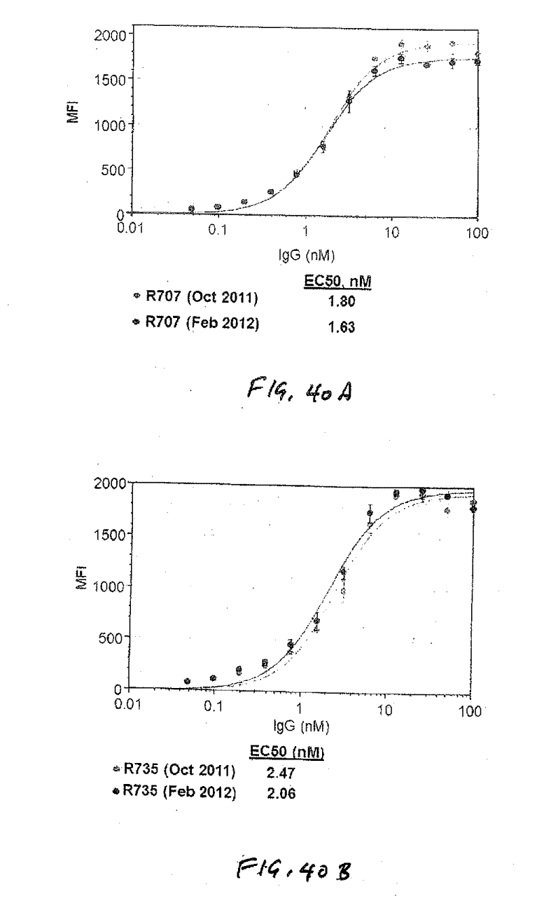

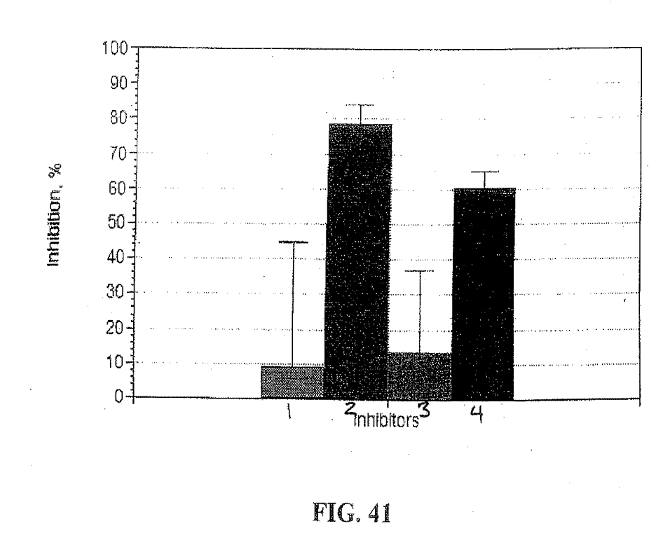

30 nM CCL19 (R&D) in serum-free cultivation media was added to lower wells of AP48 chemotaxis chamber (Neuro Probe™), cells pre-incubated with IgGs were added to upper well (10,000 cells per well). Chemotaxis was performed over a period of 3 h at 37° C. in 5% CO₂ in air. Cells that moved across 8-micrometer pores of a PRB-8 membrane to the lower well were collected, stained with PI, and live cells were counted with GUAVA PCA-96. Inhibition was calculated according to the following formula:

$$Inhibition = ( 1 - \frac{\overline{I} - \overline{B}}{\overline{C} - \overline{B}} ) \times 100 \%$$

where I—number of cells (n=6) in inhibited samples, C—number of cells (n=10) in control samples, B—number of cells moved without attractant. Results are shown in FIG. 41.

FIG. 41 depicts graphs of chemotactic activity of CLL-AAT cells in response to CCL 19. Column 1 (left) depicts chemotaxis in response to CCL19 in the presence of 500 nM R704 Human IgG1. Column 2 depicts chemotaxis in response to CCL19 in the presence of 500 nM R707 Human IgG1. Column 3 depicts chemotaxis in response to CCL19 in the presence of 500 nM R735 Human IgG1. Column 4 depicts chemotaxis in response to CCL19 in the presence of prior art antibody 500 nM 150503 Mouse IgG2 (R&D).

We conclude from this study that R704 IgG1 alone had no effect on chemotaxis toward CCL19, and R735 IgG1 did not inhibit CCL19-induced chemotaxis. We also conclude that the prior art anti-CCR7 antibody (150503 IgG had an intermediate effect (˜60% inhibition) of CCL19-induced chemotaxis. We also conclude that R707 IgG1 of this invention produced substantial (˜80% inhibition) of CCL19-induced chemotaxis. Therefore, R707 can potently inhibit chemotaxis of CLL-AAT cells, from which we conclude that R707 can inhibit metastasis of cancer cells.

### Example 32

### Inhibition of Calcium Flux II

To determine whether anti-CCR7 antibodies of this invention inhibit calcium flux induced by chemokine CCL21, we carried out a series of experiments as follows.

Chem-1 CCR7h cells were grown overnight at 37° C. in 5% CO₂ in air, in cell culture media—DMEM/F12 with G418 and 10% serum.

The cells were starved in serum-free media (CHO-S-SFM II) 3 h, 37° C., 5% CO₂, and then were loaded with dye (Calcium-5 kit) with (squares and circles) or without (triangles) IgG 30 min, 37° C., 5% CO₂. CCL19 (R&D) or CCL21 (R&D) in Tris buffered saline (TBS) was added to dye-loaded cells to reach concentrations of 15 nM and 30 nM, respectively. Inhibition was calculated as function:

$$Inhibition = ( 1 - \frac{[ \overline{I} ]}{[ \overline{C} ]} ) \times 100 \% ,$$

where I is the mean peak value in inhibited samples, C is the mean peak value in control samples.

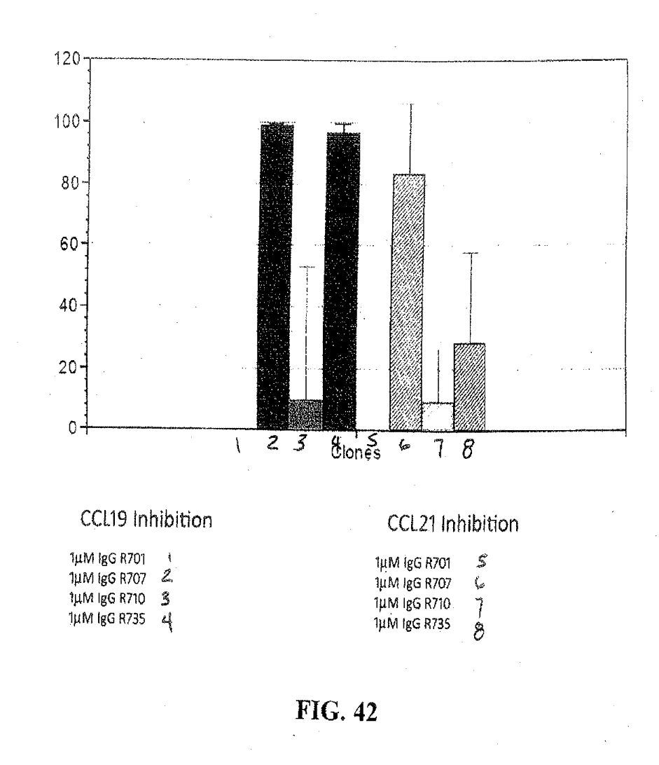

These studies demonstrated that fully human anti-CCR7 antibodies of this invention were also capable of inhibiting Ca-flux induced by CCL21. FIG. 42 depicts a. graph of these results. At concentration 1 μM, those antibodies that displayed inhibition of CCL19-induced Ca-flux were also capable of inhibiting calcium flux induced by CCL21 (FIG. 42). Columns 1-4 depict graphs of the results obtained for inhibition of calcium flux induced by CCL19: column 1 MSM R701, column 2, MSMR707, column 3, MSM R710, and column 4, MSM R735. Columns 5-8 depict graphs of results obtained for inhibition of calcium flux induced by CCL21: column 5, MSM R701, column 2, MSM R707, column 7, MSM R710, and column 8, MSM R735.

Among all tested antibodies, MSM-R701 and MSM-R710 displayed little or no inhibition of CCL19 and CCL21. MSM-R735 inhibited calcium flux induced by both chemokines, and MSM-R707, which displayed virtually complete inhibition of CCL21-induced Ca-flux (along with CCL19 induced).

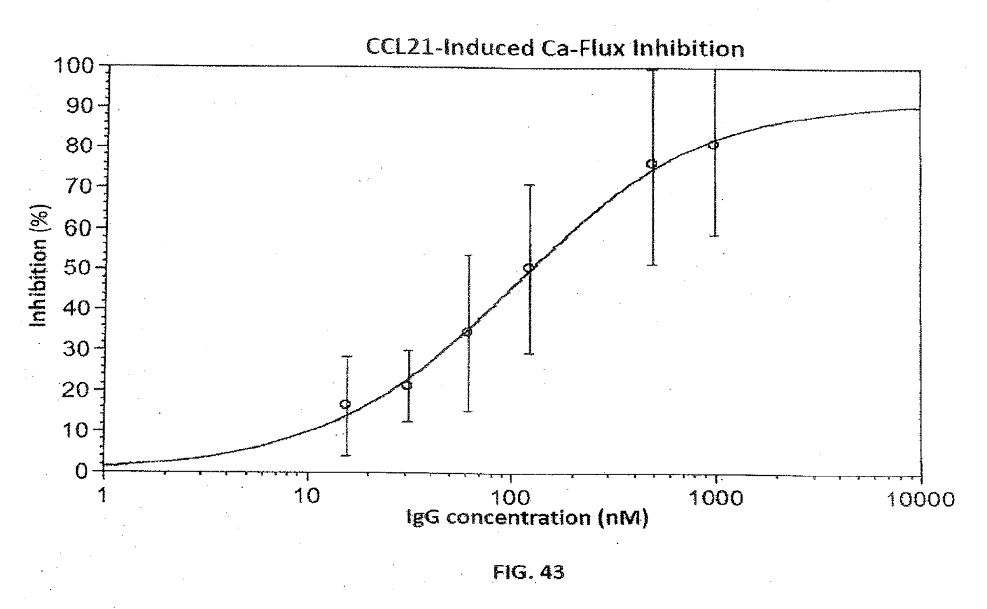

FIG. 43 depicts a graph of the effects of the concentration of MSM R707 (horizontal axis) versus the inhibition of CCL21-induced calcium flux (vertical axis). FIG. 43 shows that MSM R707 inhibited CCL21-induced calcium flux in Chem-1 CCR7h cells in a concentration-dependent fashion, with an IC50 of about 100 nM. We conclude that antibodies of the present invention can inhibit CCR7-dependent chemotaxis and other cognate CCR7 ligands-dependent processes in concentration-dependent fashions in the human body,

### Example 33

### Generation of Fully Human Anti-CCR7 Antibodies in IgG4 Format

We also produced fully human anti-CCR7 antibodies generated in other formats. In general, those skillful in the art can generate fully human antibodies in any desirable format using known sequences of germ line human antibodies in respective format and sequences of CDRs of heavy chain for an antibody binder.

MSM-R707 and MSM-R735 were generated in IgG4 format.

First, by means of molecular biology, we produced respective Heavy Chain CDR1, CDR2, and CDR3 DNA sequences into the germ line IgG4 sequences that encode fully human IgG4 antibody amino acid sequences (with the CDRs shown in bold italics) as follows in Tables 14 and 15.

TABLE 14
Sequence of R707 HC IgG4:

EVQLLESGGGLVQPGGSLRLSCAASGFTF [symbol] WVRQAPGKGLEW
VS [symbol] [symbol] VKGRFTISRDNSKNTLYLQMNSLRAEDTAVY
YCARGLTMMYTPGMDYWGQGTLVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPSCPAPEF
LGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLTCLVKGFYPS
DIAVEWESNGQPENNYKTTPPVLDSDGSFFLYSRLTVDKSRWQEGNV
FSCSVMHEALHNHYTQKSLSLSPGK SEQ ID NO. 76

TABLE 15
Sequence of R735 HC IgG4:

EVQLLESGGGLVQPGGSLRLSCAASGFTF [symbol] WVRQAPGKGLEW
VS [symbol] [symbol] SVKGRFTISRDNSKNTLYLQMNSLRAEDTAVY
YCARS [symbol] [symbol] WGQGTLVTVSSASTKGPSVFPLAPCSRSTSES
TAALGCLVKDYFPEPVTVSWNSGALTSGVHTFPAVLQSSGLYSLSSV
VTVPSSSLGTKTYTCNVDHKPSNIKVDKRVESKYGPPCPSCPAPEFL
GGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLP
SSIEKTISKAKGQPREPQVYTLPPSQEEMTKNQVSLTCLVKGFYPSD
IAVEWESNGQPENNYKTTPPVLDSDGSFFLYSRLTVDKSRWQEGNVF
SCSVMHEALHNHYTQKSLSLSPGK; SEQ ID NO. 77

Then, we transfected mammalian cells CHO, HEK, NSO, or others with the plasmids carrying the germ line light chain and respective heavy chains above, followed by antibody production in mammalian cell culture, and purification

### Example 34

### Binding Specificity of IgG4 Formatted Anti-CCR7 Antibodies

The IgG4 antibodies of this invention so generated were assayed for their specificity of binding to human CCR7 and its mouse ortholog expressed on various cells, their binding EC₅₀, Ca-flax inhibition IC₅₀, and addressed their manufacturability and stability. CCR7 over-expressing cells were incubated with 100 nM IgG in FACS buffer in 40 min at 4° C. Then cells were washed 3 times and stained with anti-human Fe antibody-PE conjugate (Jackson Immunoresearch). Cells were then washed twice and the mean fluorescence intensity (MFI) was analyzed using a GUAVA PCA-96 cell sorter at 425V (n=3).

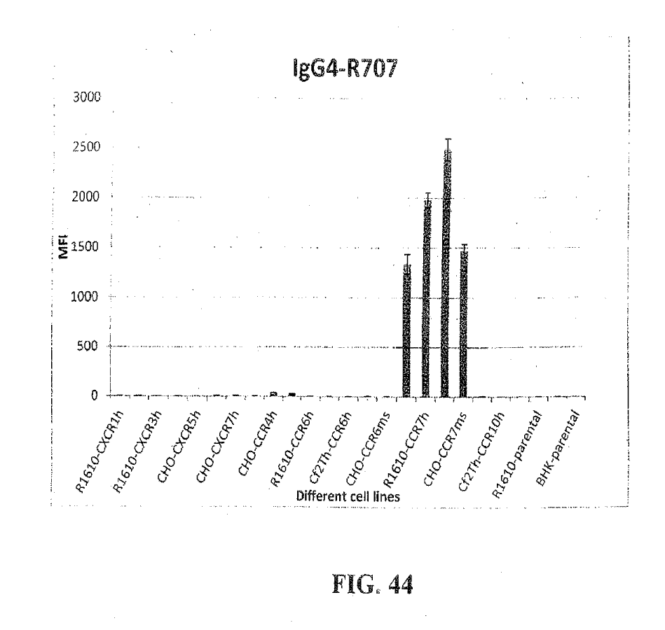

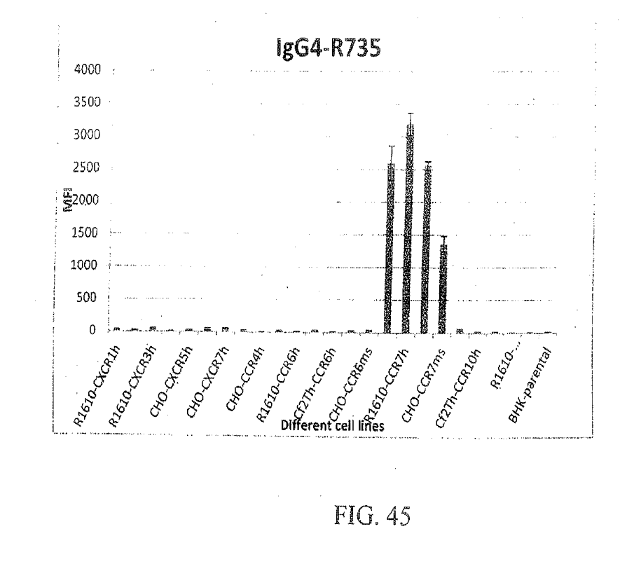

FIGS. 44 and 45 depict graphs of the specificity of binding of the fully human anti-human CCR7 antibody MSM 707 (FIG. 44) and MSM 735 (FIG. 45) in IgG4 format to cell lines expressing different GPCRs. FIG. 44 shows that the IgG4 formatted anti-CCR7 MSM R707 binds specifically to cells expressing CCR7, but none, or little binding to cells expressing CXCR1, CXCR3, CXCR5, CXCR7, CCR4, CCR6 (human or mouse), CCR1, or the parental cell lines R1620 and BHK. Thus, fully human anti-human CCR7 monoclonal antibodies in either IgG1 or IgG4 format bind selectively to CCR7. Similarly, FIG. 45 shows that IgG4 formatted MSM R735 also bound specifically to cells expressing CCR7 but not to cells expressing other GPCRs.

### Example 35

### Inhibition of CCL19-Induced Ca-Flux in Chem-1 CCR7h Cells by IgG4 Formatted Anti-CCR 7 Antibodies

To determine whether IgG4 formatted anti-CCR7 antibodies of this invention inhibit calcium flux induced by CCL19, we carried out a series of experiments similar to those used for IgG1 formatted antibodies.

To do this, Chem-1 CCR7h cells were starved in serum-free media (CHO-FreeStyle) 5 h, 37° C., 5% CO₂, and then were loaded with dye (Calcium-5 kit) with different concentration of IgG4 R707 (titering from 0.4 μM with dilution factor=2.5) 30 min, 37° C., 5% CO₂.

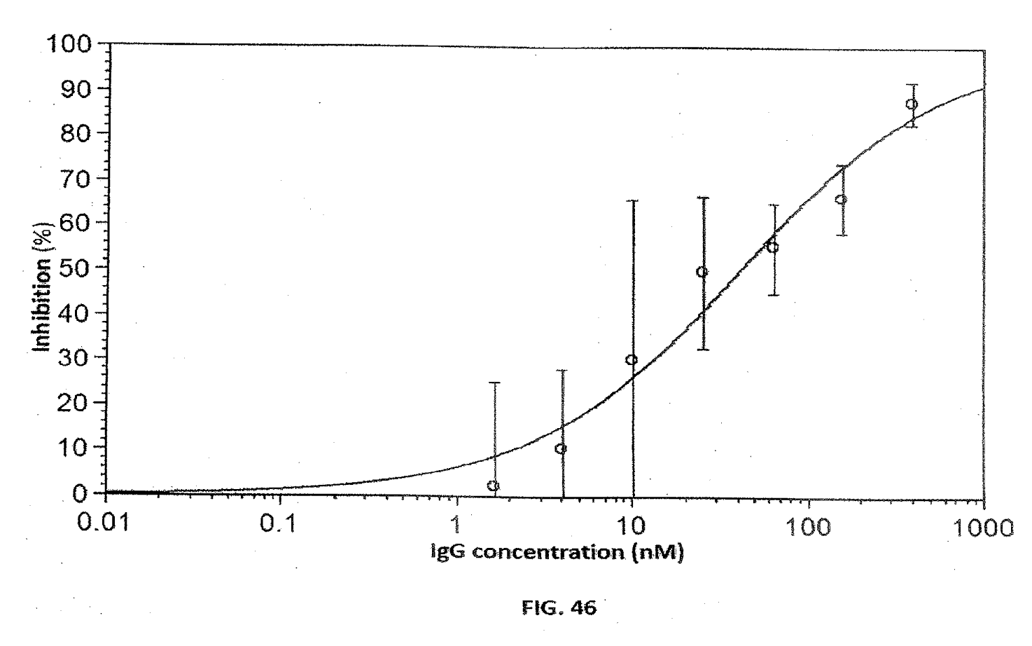

CCL19 (R&D) in TBS with 1 mM CaCl₂ was added to dye-loaded cells to reach concentrations 18 nM. Inhibition was calculated as function:

$$Inhibition = ( 1 - \frac{[ \overline{I} ]}{[ \overline{C} ]} ) \times 100 \% ,$$

where I is the mean peak value (n=4) in inhibited samples, and C is the mean peak value (n=18) in control samples. Results are shown in FIG. 46, which depicts a graph of the concentration of IgG4 formatted MSM R707 on calcium flux from cells expressing human CCR7.

As can be seen in FIG. 46, fully human anti-human CCR7 in IgG4 format inhibited CCL19-induced calcium flux, with an IC₅₀ of about 20 nM.

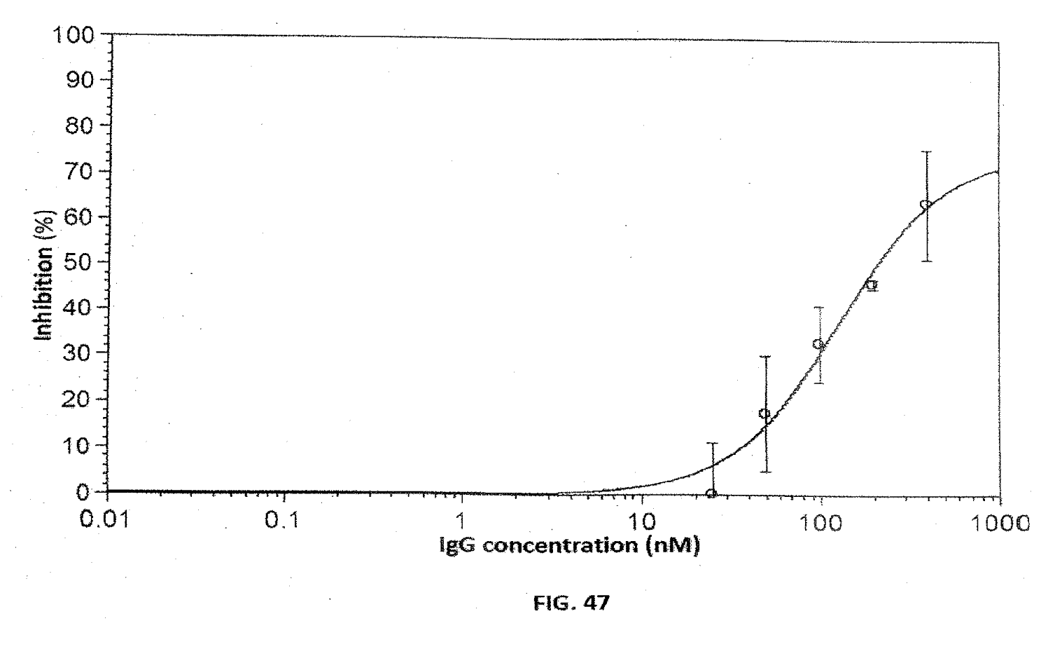

Similar results were observed in study of IgG4 formatted MSM R735. FIG. 47 depicts a graph of the concentration of IgG4 formatted MSM R735 (horizontal axis) vs. percent inhibition of calcium flux in cells expressing human CCR7. The observed IC₅₀ was 128 nM.

### Example 36

### Manufacture of IgG4 Formatted Anti-CCR7 Antibodies

To determine suitability of IgG4 formatted anti-CCR7 antibodies of this invention, we carried out a series of studies as follows:

Temperature Stress:

Samples were taken from the final bulk IgG4 antibodies in MSM-storage buffer for IgG and incubated for 12 h at 40° C.

pH Stress:

1M Citric acid was added to samples IgG4 ( 1/10V of citrate from the final volume of antibodies in MSM-storage buffer for IgG) to final pH=3.0 in 0.1M citrate and incubated at 4° C. (1 hr, 2 hr, 3 hr), then dialysis against IgG storage buffer pH=5.5 overnight.

Integrity of purified MABs was checked in non-reducing and reducing SDS PAGE before and after stress. IgG stability was checked by EC₅₀ determination before and after stress.

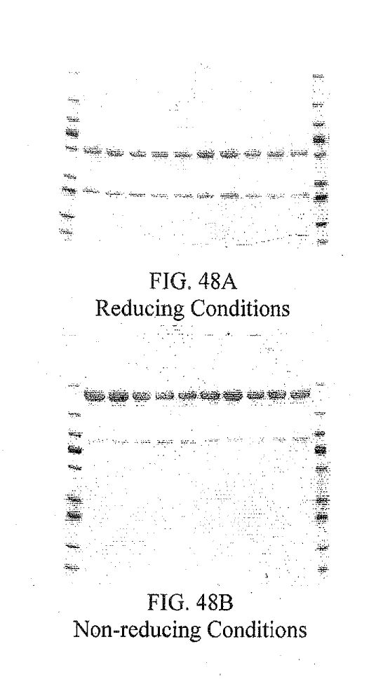

FIGS. 48A and 48B depict results of these studies. In each figure, lanes labeled “M” show molecular size markers. Lanes 1 and 6 show MSM R707 and MSM R735, respectively before stressed. Lanes 2 and 7 show results of IgG4 formatted anti-CCR7 antibodies after temperature stress. Lanes 3 and 8 show results of IgG4 formatted anti-CCR7 antibodies after pH stress for 1 hour. Lanes 4 and 9 show results after pH stress for 2 hours. Lanes 5 and 10 show results after pH stress for 3 hours.

### Example 37

### Binding of IgG4 Formatted Antibodies to CCR7 Over-Expressing and Parental Cells

To determine whether IgG4 formatted anti-CCR7 antibodies show specificity of binding to CCR7 over-expressing and parental cells, we used methods as describe above herein.

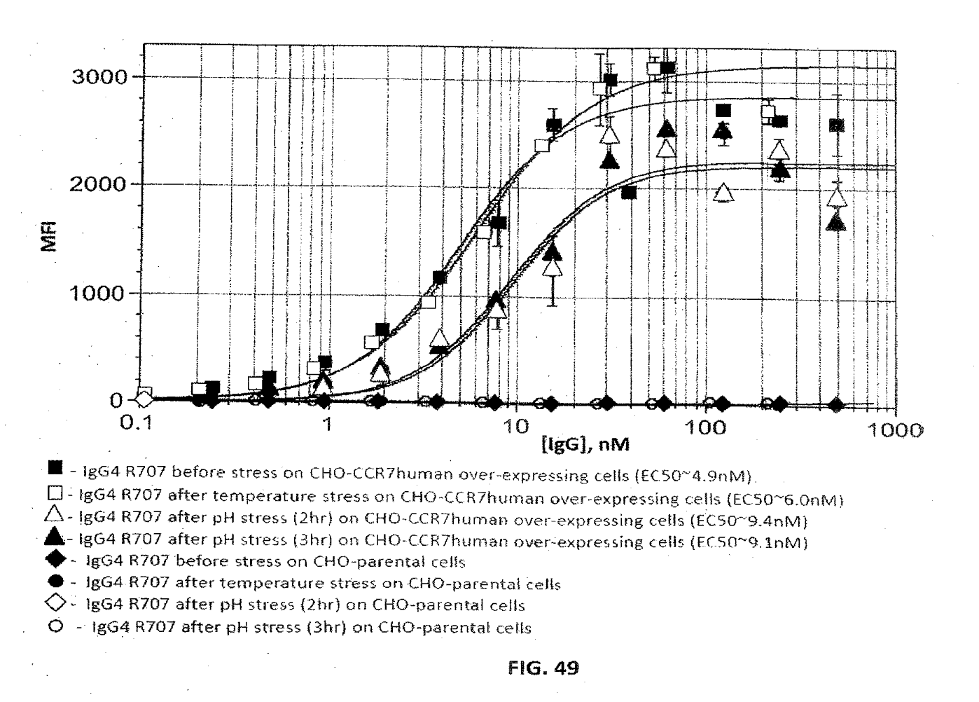

FIG. 49 depicts graphs of binding of IgG4 formatted MSM R707 antibodies of this invention. Binding to over-expressing cells had IC50s in the range of from about 4.9 nM to about 7.6 nM, whereas there was little or no binding to parental cells.

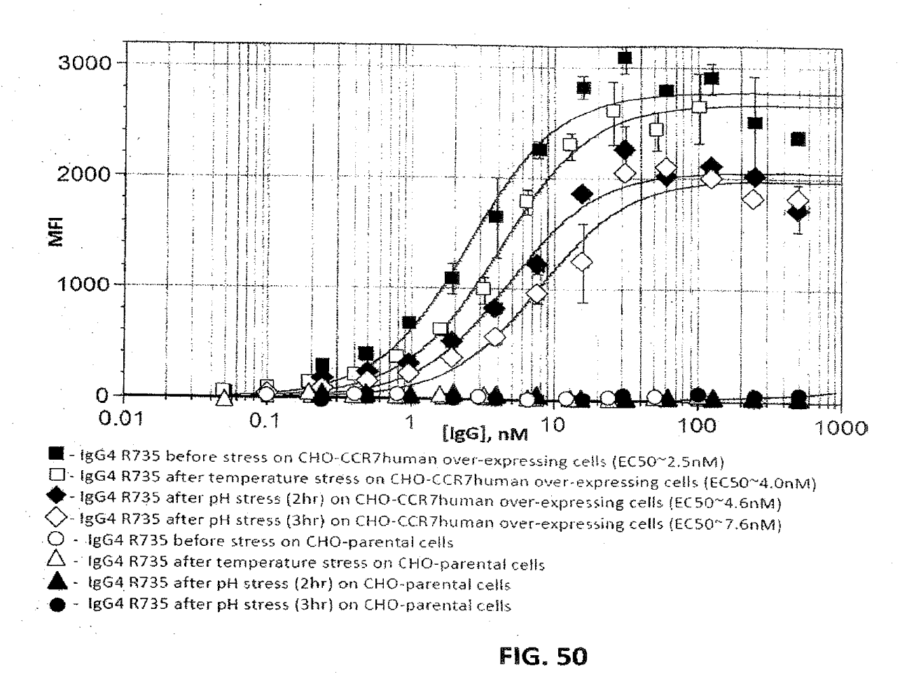

FIG. 50 depicts graphs of binding of IgG4 formatted MSM R735. As with MSM R707, IgG4 formatted antibodies of this invention bound to over-expressing cells with IC₅₀s in the range of about 2.5 nM to about 5.3 nM, whereas there was little if any binding to parental cells.

### Example 38

### Sequences and Properties of MSM R707 HC CDR3 Derivatives

One embodiment of this invention discloses derivatives of antibodies of this invention obtained by site directed mutagenesis of one or several CDR regions of the original antibodies of this invention.

Site directed mutagenesis of original DNA sequences is a well-known procedure for those skillful in the art, and so is the transfection of cells capable of antibody or FAB or scFv expression with appropriate DNA vectors carrying mutagenized gene (for scFv) or genes of heavy and light chains (for FAB or full antibodies in IgG1, IgG4, or other format) in order to produce respective antibodies. The examples of such derivatives are provided in the Table 16 below.

All mutants so produced originated from MSM-R707 IgG1 that has the VK3 A27 light chain (the germline; Table 15) and DP47 germline heavy chain. All these mutants were generated by replacing one or several amino acids in the HC CDR3 of the original MSM-R707 antibody. Regardless of the strategy of replacement, for example, one at a time in an iterative process, or several amino acids at ones, there is no way to predict whether such derivatized antibodies remain capable of recognizing the target and what are the antibody properties. However, the antibodies provided in Table 16 all were generated in IgG1 format in mammalian cells, purified, and found to be capable of binding to human CCR7-expressing cells.

While these binding antibodies represent a subset of a much larger pool of derivatives that were produced in order to identify those in Tables 16 and 17, others were found to be incapable of binding to CCR7. Any anti-CCR7 antibody or its fragment that is a CCR7 binder and has more than 80% sequence homology in HCDR3 alone or/and LCDR3+HCDR1+HCDR2 cumulatively with the existing antibody binder to the same CCR7 target represent an antibody derivative of the present invention.

TABLE 16
MSM-R707 Mutants Obtained by the Heavy Chain CDR3 Mutgenesis

| HC | HCDR1 | HCDR2 | HCDR3 |
|---|---|---|---|
| DP47 | FTFSSYAMSWVR | VSAISGSGGSTYYADS | Not applicable |
| | SEQ ID NO. 78 | SEQ ID NO. 79 | |
| R707 | FTFSNYAIHWVR | VSAITPRGGYTYYADS | CARGLTMMYTPGMDYWGQ |
| | SEQ ID NO. 80 | SEQ ID NO. 81 | SEQ ID NO. 82 |
| R707IMF | FTFSNYAIHWVR | VSAITPRGGYTYYADS | CARGLTIMYTPGFDYWGQ |
| | SEQ ID NO. 83 | SEQ ID NO. 84 | SEQ ID NO. 85 |
| R707LMF | FTFSNYAIHWVR | VSAITPRGGYTYYADS | CARGLTLMYTPGFDYWGQ |
| | SEQ ID NO. 86 | SEQ ID NO. 87 | SEQ ID NO. 88 |
| R707FMF | FTFSNYAIHWVR | VSAITPRGGYTYYADS | CARGLTFMYTPGFDYWGQ |
| | SEQ ID NO. 89 | SEQ ID NO. 90 | SEQ ID NO. 91 |
| R707WMF | FTFSNYAIHWVR | VSAITPRGGYTYYADS | CARGLTWMYTPGFDYWGQ |
| | SEQ ID NO. 92 | SEQ ID NO. 93 | SEQ ID NO. 94 |
| R707SMF | FTFSNYAIHWVR | VSAITPRGGYTYYADS | CARGLTSMYTPGFDYWGQ |
| | SEQ ID NO. 95 | SEQ ID NO. 96 | SEQ ID NO. 97 |
| R707TMF | FTFSNYAIHWVR | VSAITPRGGYTYYADS | CARGLTTMYTPGFDYWGQ |
| | SEQ ID NO. 98 | SEQ ID NO. 99 | SEQ ID NO. 100 |
| R707NMF | FTFSNYAIHWVR | VSAITPRGGYTYYADS | CARGLTNMYTPGFDYWGQ |
| | SEQ ID NO. 101 | SEWQ ID NO. 102 | SEQ ID NO. 103 |
| R707KMF | FTFSNYAIHWVR | VSAITPRGGYTYYADS | CARGLTKMYTPGFDYWGQ |
| | SEQ ID NO. 104 | SEQ ID NO. 105 | SE ID NO. 106 |
| R707RMF | FTFSNYAIHWVR | VSAITPRGGYTYYADS | CARGLTRMYTPGFDYWGQ |
| | SEQ ID NO. 107 | SEQ ID NO. 108 | SEQ ID NO. 109 |
| R707MLF | FTFSNYAIHWVR | VSAITPRGGYTYYADS | CARGLTMLYTPGFDYWGQ |
| | SEQ ID NO. 110 | SEQ ID NO. 111 | SEQ ID NO. 112 |
| R707MVF | FTFSNYAIHWVR | VSAITPRGGYTYYADS | CARGLTMVYTPGFDYWGQ |
| | SEQ ID NO. 113 | SEQ ID NO. 114 | SEQ ID NO. 115 |
| R707MAF | FTFSNYAIHWVR | VSAITPRGGYTYYADS | CARGLTMAYTPGFDYWGQ |
| | SEQ ID NO. 116 | SEQ ID NO. 117 | SE ID NO. 118 |
| R707MGF | FTFSNYAIHWVR | VSAITPRGGYTYYADS | CARGLTMGYTPGFDYWGQ |
| | SEQ ID NO. 119 | SEQ ID NO. 120 | SEQ ID NO. 121 |
| R707MSF | FTFSNYAIHWVR | VSAITPRGGYTYYADS | CARGLTMSYTPGFDYWGQ |
| | SEQ ID NO. 122 | SEQ ID NO. 123 | SEQ ID NO. 124 |
| R707MTF | FTFSNYAIHWVR | VSAITPRGGYTYYADS | CARGLTMTYTPGFDYWGQ |
| | SEQ ID NO. 125 | SEQ ID NO. 126 | SEQ ID NO. 127 |
| R707MQF | FTFSNYAIHWVR | VSAITPRGGYTYYADS | CARGLTMQYTPGFDYWGQ |
| | SEQ ID NO. 128 | SEQ ID NO. 129 | SEQ ID NO. 130 |
| R707MPF | FTFSNYAIHWVR | VSAITPRGGYTYYADS | CARGLTMPYTPGFDYWGQ |
| | SEQ ID NO. 131 | SEQ ID NO. 132 | SE ID NO. 133 |
| R707MRF | FTFSNYAIHWVR | VSAITPRGGYTYYADS | CARGLTMRYTPGFDYWGQ |
| | SEQ ID NO. 134 | SEQ ID NO. 135 | SEQ ID NO. 13.6 |
| R707B | FTFSNYAIHWVR | VSAITPRGGYTYYADS | CARGLTYSYTPGFDYWGQ |
| | SEQ ID NO. 137 | SEQ ID NO. 138 | SEQ ID NO. 139 |
| R707BI | FTFSNYAIHWVR | VSAITPRGGYTYYADS | CARGLTISYTPGFDYWGQ |
| | SEQ ID NO. 140 | SEQ ID NO. 141 | SEQ ID NO. 142 |
| R707BL | FTFSNYAIHWVR | VSAITPRGGYTYYADS | CARGLTLMYTPGFDYWGQ |
| | SEQ ID NO. 143 | SEQ ID NO. 144 | SEQ ID NO. 145 |
| R707BR | FTFSNYAIHWVR | VSAITPRGGYTYYADS | CARGLTRSYTPGFDYWGQ |
| | SEQ ID NO. 146 | SEQ ID NO. 147 | SEQ ID NO. 148 |

TABLE 17
Light Chain CDRs of the Antibodies of This Invention

| LC | LCCDR1 | LCCDR2 | LCCDR3 | Features |
|---|---|---|---|---|
| VK3 A27 | RASQSVSSSYLA | GASSRAT | CQQSYSSPITFGQ | germline |
| | SEQ ID NO. 149 | SEQ ID NO. 150 | SEQ ID NO. 151 | |
| R707 | RASQSVSSSYLA | GASSRAT | CQQSYSSPITFGQ | germline |
| | SEQ ID NO. 152 | SEQ ID NO. 153 | SEQ ID NO. 154 | |

### INCORPORATION BY REFERENCE

All of the patent applications, patents, and non-patent literature cited herein are fully incorporated by reference as if separately so incorporated.

### INDUSTRIAL APPLICATION

Fully human antibodies against human CCR7 can be used in the manufacture of compositions that are useful to treat a variety of disorders in humans and other mammals. The antibodies of this invention can also be used to diagnose disorders characterized by abnormal expression or action of CCR7.

1-9. (canceled)

10. An antibody selected from the group consisting of MSM R707, MSM R707B, MSM R707BR, MSM R707BL, MSM R707 BI, MSM R710, and MSM R735.

11. The antibody of claim 10, where the VH sequence is encoded by SEQ ID NO.3, and the VL region is encoded by SEQ ID NO.4.

12. The antibody of claim 10, where the HC amino acid sequence is SEQ ID NO.5, and the LC sequence is SEQ ID NO.6.

13. The antibody of claim 10, where the CDR1 HC has the amino acid sequence of SEQ ID NO.7, the CDR2 HC has the amino acid sequence of SEQ ID NO.8, the CDR3 HC has the amino acid sequence of SEQ NO.9, the CDR1 LC has the amino acid sequence of SEQ ID NO.10, the CDR2 LC has the amino acid sequence of SEQ ID NO.11, and the CDR3 LC has the amino acid sequence of SEQ ID NO.12.

14. The antibody of claim 10, where the VH sequence is encoded by SEQ ID NO.13, and the VL region is encoded by SEQ ID NO.14.

15. The antibody of claim 10, where the HC amino acid sequence is SEQ ID NO.15, and the LC amino acid sequence is SEQ ID NO.16.

16. The antibody of claim 10, where the CDR1 HC has the amino acid sequence of SEQ ID NO.17, the CDR2 HC has the amino acid sequence of SEQ ID NO.18, the CDR3 HC has the amino acid sequence of SEQ NO.19, the CDR1 LC has the amino acid sequence of SEQ ID NO.20, the CDR2 LC has the amino acid sequence of SEQ ID NO.21, and the CDR3 LC has the amino acid sequence of SEQ ID NO.22.

17. The antibody of claim 10, where the VH sequence is encoded by SEQ ID NO.23, and the VL region is encoded by SEQ ID NO.24.

18. The antibody of claim 10, where the HC amino acid sequence is SEQ ID NO.25, and the LC amino acid sequence is SEQ ID NO.26.

19. The antibody of claim 10, where the CDR1 HC has the amino acid sequence of SEQ ID NO.27, the CDR2 HC has the amino acid sequence of SEQ ID NO.28, the CDR3 HC has the amino acid sequence of SEQ NO.29, the CDR1 LC has the amino acid sequence of SEQ ID NO.30, the CDR2 LC has the amino acid sequence of SEQ ID NO.31, and the CDR3 LC has the amino acid sequence of SEQ ID NO.32.

20. The antibody of claim 10, where the VH sequence is encoded by SEQ ID NO.33, and the VL region is encoded by SEQ ID NO.34.

21. The antibody of claim 10, where the HC amino acid sequence is SEQ ID NO.35, and the LC amino acid sequence is SEQ ID NO.36.

22. The antibody of claim 10, where the CDR1 HC has the amino acid sequence of SEQ ID NO.37, the CDR2 HC has the amino acid sequence of SEQ ID NO.38, the CDR3 HC has the amino acid sequence of SEQ NO.39, the CDR1 LC has the amino acid sequence of SEQ ID NO.40, the CDR2 LC has the amino acid sequence of SEQ ID NO.41, and the CDR3 LC has the amino acid sequence of SEQ ID NO.42.

23. The antibody of claim 10, where the VH sequence is encoded by SEQ ID NO.43, and the VL region is encoded by SEQ ID NO.44.

24. The antibody of claim 10, where the HC amino acid sequence is SEQ ID NO.45, and the LC amino acid sequence is SEQ ID NO.46.

25. The antibody of claim 10, where the CDR1 HC has the amino acid sequence of SEQ ID NO.47, the CDR2 HC has the amino acid sequence of SEQ ID NO.48, the CDR3 HC has the amino acid sequence of SEQ NO.49, the CDR1 LC has the amino acid sequence of SEQ ID NO.50, the CDR2 LC has the amino acid sequence of SEQ ID NO.51, and the CDR3 LC has the amino acid sequence of SEQ ID NO.52.

26. The antibody of claim 10, where the VH sequence is encoded by SEQ ID NO.53, and the VL region is encoded by SEQ ID NO.54.

27. The antibody of claim 10, where the HC amino acid sequence is SEQ ID NO.55, and the LC amino acid sequence is SEQ ID NO.56.

28. The antibody of claim 10, where the CDR1 HC has the amino acid sequence of SEQ ID NO.57, the CDR2 HC has the amino acid sequence of SEQ ID NO.58, the CDR3 HC has the amino acid sequence of SEQ NO.59, the CDR1 LC has the amino acid sequence of SEQ ID NO.60, the CDR2 LC has the amino acid sequence of SEQ ID NO.61, and the CDR3 LC has the amino acid sequence of SEQ ID NO.62.

29. The antibody of claim 10, where the VH sequence is encoded by SEQ ID NO.63, and the VL region is encoded by SEQ ID NO.64.

30. The antibody of claim 10, where the HC amino acid sequence is SEQ ID NO.65, and the LC amino acid sequence is SEQ ID NO.66.

31-47. (canceled)


SEQUENCE LISTING (168 sequences)

SEQ ID NO: 1 - DNA, 13 bp, Unknown
  Kozak consensus sequence
gccgccaccatgg

SEQ ID NO: 2 - protein, 9 aa, Bos taurus
  rhodopsin C9 peptide tag
TETSQVAPA

SEQ ID NO: 3 - DNA, 363 bp, Artificial Sequence
  IgG1 Heavy Chain MRM R707
gaagttcaactgctggagtccggtggtggtctggtacagccgggtggttctctgcgtctgagttgcgcggccagtggctttaccttcagtaactatgcgatccattgggtgcgtcaggctccgggcaaaggtctggaatgggttagcgctattactccgaggggtggctatacctactatgcggatagcgtgaaaggccgttttaccatttctcgcgacaacagcaagaacacgctgtacctgcagatgaactcactgcgtgccgaagatacggccgtgtattactgtgcgagaggcctgacgatgatgtacactcccggcatggactactggggccagggaaccttggtcaccgtctcgagt

SEQ ID NO: 4 - DNA, 324 bp, Artificial Sequence
  IgG1 Light Chain MSM R707
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatcttattcttctcctatcacgttcggccaagggaccaaggtggaaatcaaa

SEQ ID NO: 5 - protein, 451 aa, Artificial Sequence
  IgG1 Heavy Chain MSM R707
EVQLLESGGGLVQPGGSLRLSCAASGFTFSNYAIHWVRQAPGKGLEWVSAITPRGGYTYYADSVKGRFTISRDNSKNTLYLQMNSLRAEDTAVYYCARGLTMMYTPGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 215 aa, Artificial Sequence
  IgG1 Light Chain MSM R707
EIVLTQSPGTLSLSPGERATLSCRASQSVSSSYLAWYQQKPGQAPRLLIYGASSRATGIPDRFSGSGSGTDFTLTISRLEPEDFAVYYCQQSYSSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 5 aa, Artificial Sequence
  IgG1 CDR1 Heavy Chain MSM R707
NYAIH

SEQ ID NO: 8 - protein, 17 aa, Artificial Sequence
  IgG1 CDR2 Heavy Chain MSM R707
AITPRGGYTYYADSVKG

SEQ ID NO: 9 - protein, 12 aa, Artificial Sequence
  IgG1 CDR3 Heavy Chain MSM R707
GLTMMYTPGMDY

SEQ ID NO: 10 - protein, 12 aa, Artificial Sequence
  IgG1 CDR1 Light Chain MSM R707
RASQSVSSSYLA

SEQ ID NO: 11 - protein, 7 aa, Artificial Sequence
  IgG1 CDR2 Light Chain MSM R707
GASSRAT

SEQ ID NO: 12 - protein, 9 aa, Artificial Sequence
  IgG1 CDR3 Light Chain MSM R707
QQSYSSPIT

SEQ ID NO: 13 - DNA, 363 bp, Artificial Sequence
  IgG1 Heavy Chain MSM R707B
gaagttcaactgctggagtccggtggtggtctggtacagccgggtggttctctgcgtctgagttgcgcggccagtggctttaccttcagtaactatgcgatccattgggtgcgtcaggctccgggcaaaggtctggaatgggttagcgctattactccgaggggtggctatacctactatgcggatagcgtgaaaggccgttttaccatttctcgcgacaacagcaagaacacgctgtacctgcagatgaactcactgcgtgccgaagatacggccgtgtattactgtgcgagaggcctgacgtactcttacactcccggctttgactactggggccagggaaccttggtcaccgtctcgagt

SEQ ID NO: 14 - DNA, 324 bp, Artificial Sequence; identical to SEQ ID NO: 4
  IgG1 Light Chain MSM R707B

SEQ ID NO: 15 - protein, 451 aa, Artificial Sequence
  IgG1 Heavy Chain MSM R707B
EVQLLESGGGLVQPGGSLRLSCAASGFTFSNYAIHWVRQAPGKGLEWVSAITPRGGYTYYADSVKGRFTISRDNSKNTLYLQMNSLRAEDTAVYYCARGLTYSYTPGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 451 aa, Artificial Sequence; identical to SEQ ID NO: 15
  IgG1 Light Chain MSM R707B

SEQ ID NO: 17 - protein, 215 aa, Artificial Sequence; identical to SEQ ID NO: 6
  IgG1 CDR1 Heavy Chain MSM R707B

SEQ ID NO: 18 - protein, 17 aa, Artificial Sequence
  IgG1 CDR2 Heavy Chain MSM R707B
AITPRGGYTYYADSVKG

SEQ ID NO: 19 - protein, 12 aa, Artificial Sequence
  IgG1 CDR3 Heavy Chain MSM R707B
GLTYSYTPGFDY

SEQ ID NO: 20 - protein, 12 aa, Artificial Sequence
  IgG1 CDR1 Light Chain MSM R707B
RASQSVSSSYLA

SEQ ID NO: 21 - protein, 7 aa, Artificial Sequence
  IgG1 CDR2 Light Chain MSM R707B
GASSRAT

SEQ ID NO: 22 - protein, 9 aa, Artificial Sequence
  IgG1 CDR3 Light Chain MSM R707B
QQSYSSPIT

SEQ ID NO: 23 - DNA, 363 bp, Artificial Sequence
  IgG1 Heavy Chain MSM R707BR
gaagttcaactgctggagtccggtggtggtctggtacagccgggtggttctctgcgtctgagttgcgcggccagtggctttaccttcagtaactatgcgatccattgggtgcgtcaggctccgggcaaaggtctggaatgggttagcgctattactccgaggggtggctatacctactatgcggatagcgtgaaaggccgttttaccatttctcgcgacaacagcaagaacacgctgtacctgcagatgaactcactgcgtgccgaagatacggccgtgtattactgtgcgagaggcctgacgcgctcttacactcccggctttgactactggggccagggaaccttggtcaccgtctcgagt

SEQ ID NO: 24 - DNA, 324 bp, Artificial Sequence; identical to SEQ ID NO: 4
  IgG1 Light Chain MSM R707BR

SEQ ID NO: 25 - protein, 451 aa, Artificial Sequence
  IgG1 Heavy Chain MSM R707BR
EVQLLESGGGLVQPGGSLRLSCAASGFTFSNYAIHWVRQAPGKGLEWVSAITPRGGYTYYADSVKGRFTISRDNSKNTLYLQMNSLRAEDTAVYYCARGLTRSYTPGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 215 aa, Artificial Sequence; identical to SEQ ID NO: 6
  IgG1 Light Chaim MSM R707BR

SEQ ID NO: 27 - protein, 5 aa, Artificial Sequence
  IgG1 CDR1 Heavy Chain MSM R707BR
NYAIH

SEQ ID NO: 28 - protein, 17 aa, Artificial Sequence
  IgG1 CDR2 Heavy Chain MSM R707BR
AITPRGGYTYYADSVKG

SEQ ID NO: 29 - protein, 12 aa, Artificial Sequence
  IgG1 CDR3 Heavy Chain MSM R707BR
GLTRSYTPGFDY

SEQ ID NO: 30 - protein, 12 aa, Artificial Sequence
  IgG1 CDR1 Light Chain MSM R707BR
RASQSVSSSYLA

SEQ ID NO: 31 - protein, 7 aa, Artificial Sequence
  IgG1 CDR2 Light Chain MSM R707BR
GASSRAT

SEQ ID NO: 32 - protein, 9 aa, Artificial Sequence
  IgG1 CDR3 Light Chain MSM R707BR
QQSYSSPIT

SEQ ID NO: 33 - DNA, 363 bp, Artificial Sequence
  IgG1 Heavy CHain MSM R707BL
gaagttcaactgctggagtccggtggtggtctggtacagccgggtggttctctgcgtctgagttgcgcggccagtggctttaccttcagtaactatgcgatccattgggtgcgtcaggctccgggcaaaggtctggaatgggttagcgctattactccgaggggtggctatacctactatgcggatagcgtgaaaggccgttttaccatttctcgcgacaacagcaagaacacgctgtacctgcagatgaactcactgcgtgccgaagatacggccgtgtattactgtgcgagaggcctgacgctgtcttacactcccggctttgactactggggccagggaaccttggtcaccgtctcgagt

SEQ ID NO: 34 - DNA, 324 bp, Artificial Sequence; identical to SEQ ID NO: 4
  IgG1 Light Chain MSM R707BL

SEQ ID NO: 35 - protein, 451 aa, Artificial Sequence
  IgG1 Heavy Chain MSM R707BL
EVQLLESGGGLVQPGGSLRLSCAASGFTFSNYAIHWVRQAPGKGLEWVSAITPRGGYTYYADSVKGRFTISRDNSKNTLYLQMNSLRAEDTAVYYCARGLTLSYTPGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 215 aa, Artificial Sequence; identical to SEQ ID NO: 6
  IgG1 Light Chain MSM R707BL

SEQ ID NO: 37 - protein, 5 aa, Artificial Sequence
  IgG1 CDR1 Heavy Chain MSM R707BL
NYAIH

SEQ ID NO: 38 - protein, 17 aa, Artificial Sequence
  IgG1 CDR2 Heavy Chain MSM R707BL
AITPRGGYTYYADSVKG

SEQ ID NO: 39 - protein, 12 aa, Artificial Sequence
  IgG1 CDR3 Heavy Chain MSM R707BL
GLTLSYTPGFDY

SEQ ID NO: 40 - protein, 12 aa, Artificial Sequence
  Human CDR1 LC AA MSM R707BL
RASQSVSSSYLA

SEQ ID NO: 41 - protein, 7 aa, Artificial Sequence
  IgG1 CDR2 Light Clain MSM R707BL
GASSRAT

SEQ ID NO: 42 - protein, 9 aa, Artificial Sequence
  IgG1 CDR3 Light Chain MSM R707BL
QQSYSSPIT

SEQ ID NO: 43 - DNA, 363 bp, Artificial Sequence
  IgG1 Heavy Chain MSM R707BI
gaagttcaactgctggagtccggtggtggtctggtacagccgggtggttctctgcgtctgagttgcgcggccagtggctttaccttcagtaactatgcgatccattgggtgcgtcaggctccgggcaaaggtctggaatgggttagcgctattactccgaggggtggctatacctactatgcggatagcgtgaaaggccgttttaccatttctcgcgacaacagcaagaacacgctgtacctgcagatgaactcactgcgtgccgaagatacggccgtgtattactgtgcgagaggcctgacgatctcttacactcccggctttgactactggggccagggaaccttggtcaccgtctcgagt

SEQ ID NO: 44 - DNA, 324 bp, Artificial Sequence; identical to SEQ ID NO: 4
  IgG1 Light Chain MSM R707BI

SEQ ID NO: 45 - protein, 451 aa, Artificial Sequence
  IgG1 Heavy Chain MSM R7070BI
EVQLLESGGGLVQPGGSLRLSCAASGFTFSNYAIHWVRQAPGKGLEWVSAITPRGGYTYYADSVKGRFTISRDNSKNTLYLQMNSLRAEDTAVYYCARGLTISYTPGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 215 aa, Artificial Sequence; identical to SEQ ID NO: 6
  IgG1 Light Chain MSM R707BI

SEQ ID NO: 47 - protein, 5 aa, Artificial Sequence
  IgG1 CDR1 Heavy Chain MSM R707BI
NYAIH

SEQ ID NO: 48 - protein, 17 aa, Artificial Sequence
  IgG1 CDR2 Heavy Chain MSM R707BI
AITPRGGYTYYADSVKG

SEQ ID NO: 49 - protein, 12 aa, Artificial Sequence
  IgG1 CDR3 Heavy Chain MSM R707BI
GLTISYTPGFDY

SEQ ID NO: 50 - protein, 12 aa, Artificial Sequence
  IgG1 CDR1 Light Chain MSM R707BI
RASQSVSSSYLA

SEQ ID NO: 51 - protein, 7 aa, Artificial Sequence
  IgG1 CDR2 Light Chain MSM R707BI
GASSRAT

SEQ ID NO: 52 - protein, 9 aa, Artificial Sequence
  IgG1 CDR3 Light Chain MSM R707BI
QQSYSSPIT

SEQ ID NO: 53 - DNA, 360 bp, Artificial Sequence
  IgG1 Heavy Chain MSM R710
gaagttcaactgctggagtccggtggtggtctggtacagccgggtggttctctgcgtctgagttgcgcggccagtggctttaccttcagtaactatacgatgcattgggtgcgtcaggctccgggcaaaggtctggaatgggttagcgggattggtccgaggagtggcaggacctactatgcggatagcgtgaaaggccgttttaccatttctcgcgacaacagcaagaacacgctgtacctgcagatgaactcactgcgtgccgaagatacggccgtgtattactgtgcgagatcttacgcttaccagtaccgtggcttcgactactggggccagggaaccttggtcaccgtctcgagt

SEQ ID NO: 54 - DNA, 312 bp, Artificial Sequence
  IgG1 Light Chain MSM R710
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagtcttctgtcacgttcggccaagggaccaaggtggaaatcaaa

SEQ ID NO: 55 - protein, 450 aa, Artificial Sequence
  IgG1 Heavy Chain MSM R710
EVQLLESGGGLVQPGGSLRLSCAASGFTFSNYTMHWVRQAPGKGLEWVSGIGPRSGRTYYADSVKGRFTISRDNSKNTLYLQMNSLRAEDTAVYYCARSYAYQYRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 211 aa, Artificial Sequence
  IgG1 Light Chain MSM R710
EIVLTQSPGTLSLSPGERATLSCRASQSVSSSYLAWYQQKPGQAPRLLIYGASSRATGIPDRFSGSGSGTDFTLTISRLEPEDFAVYYCQSSVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 5 aa, Artificial Sequence
  IgG1 CDR1 Heavy Chain MSM R710
NYTMH

SEQ ID NO: 58 - protein, 17 aa, Artificial Sequence
  IgG1 CDR2 Heavy Chain MSM R710
GIGPRSGRTYYADSVKG

SEQ ID NO: 59 - protein, 11 aa, Artificial Sequence
  IgG1 CDR3 Heavy Chain MSM R710
SYAYQYRGFDY

SEQ ID NO: 60 - protein, 12 aa, Artificial Sequence
  IgG1 CDR1 Light Chain MSM R710
RASQSVSSSYLA

SEQ ID NO: 61 - protein, 7 aa, Artificial Sequence
  IgG1 CDR2 Light Chain MSM R710
GASSRAT

SEQ ID NO: 62 - protein, 5 aa, Artificial Sequence
  IgG1 CDR3 Light Chain MSM R710
QSSVT

SEQ ID NO: 63 - DNA, 360 bp, Artificial Sequence
  IgG1 Heavy Chain MSM R735
gaagttcaactgctggagtccggtggtggtctggtacagccgggtggtcctctgcgtctgagttgcgcggccagtggctttaccttcagtaactataatatgcattgggtgcgtcaggctccgggcaaaggtctggaatgggttagcgggattgggccgcgtcggggccggacctattatgcggatagcgtgaaaggccgttttaccatttctcgcgacaacagcaagaacacgctgtacctgcagatgaactcactgcgtgccgaagatacggccgtgtattactgtgcgagatcttacgcttaccagtaccgtggcttggactactggggccagggaaccctggtcaccgtctcgagt

SEQ ID NO: 64 - DNA, 318 bp, Artificial Sequence
  IgG1 Light Chain MSM R735
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaaggtagtcctgtcacgttcggccaagggaccaaggtggaaatcaaa

SEQ ID NO: 65 - protein, 450 aa, Artificial Sequence
  IgG1 Heavy Chain MSM R735
EVQLLESGGGLVQPGGSLRLSCAASGFTFSNYNMHWVRQAPGKGLEWVSGIGPRRGRTYYADSVKGRFTISRDNSKNTLYLQMNSLRAEDTAVYYCARSYAYQYRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 213 aa, Artificial Sequence
  IgG1 Light Chain MSM R735
EIVLTQSPGTLSLSPGERATLSCRASQSVSSSYLAWYQQKPGQAPRLLIYGASSRATGIPDRFSGSGSGTDFTLTISRLEPEDFAVYYCQQGSPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 5 aa, Artificial Sequence
  IgG1 CDR1 Heavy Chain MSM R735
NYNMH

SEQ ID NO: 68 - protein, 17 aa, Artificial Sequence
  IgG1 CDR2 Heavy Chain MSM R735
GIGPRRGRTYYADSVKG

SEQ ID NO: 69 - protein, 11 aa, Artificial Sequence
  IgG1 CDR3 Heavy Chain MSM R735
SYAYQYRGLDY

SEQ ID NO: 70 - protein, 12 aa, Artificial Sequence
  IgG1 CDR1 Light Chain MSM R735
RASQSVSSSYLA

SEQ ID NO: 71 - protein, 7 aa, Artificial Sequence
  IgG1 CDR2 Light Chain MSM R735
GASSRAT

SEQ ID NO: 72 - protein, 7 aa, Artificial Sequence
  IgG1 CDR3 Light Chain MSM R735
QQGSPVT

SEQ ID NO: 73 - protein, 378 aa, Homo sapiens
  Synthetic, mammalian cell expression optimized, human CCR7 gene (encodes the human CCR7 amino acid sequence of 378 amino acids; the Swissprot accession number P32248
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 74 - protein, 38 aa, Artificial Sequence
  Synthetic streptavidin tag
MDEKTTGWRGGHVVEGLAGELEQLRARLEHHPQGQREP

SEQ ID NO: 75 - protein, 15 aa, Artificial Sequence
  S tag
KETAAAKFERQHMDS

SEQ ID NO: 76 - protein, 448 aa, Artificial Sequence
  IgG4 Heavy Chain MSM R707
  IgG4 CDR1 Heavy Chain MSM R707
  IgG4 CDR2 Heavy Chain MSM R707
  IgG4 CDR3 Heavy Chain MSM R707
EVQLLESGGGLVQPGGSLRLSCAASGFTFSNYAIHWVRQAPGKGLEWVSAITPRGGYTYYADSVKGRFTISRDNSKNTLYLQMNSLRAEDTAVYYCARGLTMMYTPGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 77 - protein, 447 aa, Artificial Sequence
  IgG4 Heavy Chain MSM R737
  IgG4 CDR1 Heavy Chain MSM R737
  IgG4 CDR2 Heavy Chain MSM R737
  IgG4 CDR3 Heavy Chain MSM R737
EVQLLESGGGLVQPGGSLRLSCAASGFTFSNYNMHWVRQAPGKGLEWVSGIGPRRGRTYYADSVKGRFTISRDNSKNTLYLQMNSLRAEDTAVYYCARSYAYQYRGLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 78 - protein, 12 aa, Artificial Sequence
  CDR1 DP47
FTFSSYAMSWVR

SEQ ID NO: 79 - protein, 16 aa, Artificial Sequence
  CDR2 DP47
VSAISGSGGSTYYADS

SEQ ID NO: 80 - protein, 12 aa, Artificial Sequence
  CDR1 Heavy Chain R707 mutant
FTFSNYAIHWVR

SEQ ID NO: 81 - protein, 16 aa, Artificial Sequence
  CDR2 Heavy Chain MSM R707 mutant
VSAITPRGGYTYYADS

SEQ ID NO: 82 - protein, 18 aa, Artificial Sequence
  CDR3 heavy Chain MSM R707 mutant
CARGLTMMYTPGMDYWGQ

SEQ ID NO: 83 - protein, 12 aa, Artificial Sequence
  CDR1 Heavy Chain MSM R707IMF mutant
FTFSNYAIHWVR

SEQ ID NO: 84 - protein, 16 aa, Artificial Sequence
  CDR2 Heavy Chain MSM R707IMF mutant
VSAITPRGGYTYYADS

SEQ ID NO: 85 - protein, 18 aa, Artificial Sequence
  CDR3 Heavy Chain MSM R707IMF mutant
CARGLTIMYTPGFDYWGQ

SEQ ID NO: 86 - protein, 12 aa, Artificial Sequence
  CDR1 Heavy Chain MSM R707LMF mutant
FTFSNYAIHWVR

SEQ ID NO: 87 - protein, 16 aa, Artificial Sequence
  IgG4 CDR2 Heavy Chain MSM R707LMF mutant
VSAITPRGGYTYYADS

SEQ ID NO: 88 - protein, 18 aa, Artificial Sequence
  CDR3 Heavy Chain MSM R707LMF mutant
CARGLTLMYTPGFDYWGQ

SEQ ID NO: 89 - protein, 12 aa, Artificial Sequence
  CDR1 Heavy Chain MSM R707FMF mutant
FTFSNYAIHWVR

SEQ ID NO: 90 - protein, 16 aa, Artificial Sequence
  CDR2 Heavy Chain MSM R707FMF mutant
VSAITPRGGYTYYADS

SEQ ID NO: 91 - protein, 18 aa, Artificial Sequence
  CDR3 Heavy Chain MSM R707FMF mutant
CARGLTFMYTPGFDYWGQ

SEQ ID NO: 92 - protein, 12 aa, Artificial Sequence
  CDR1 Heavy Chain MSM R707WMF mutant
FTFSNYAIHWVR

SEQ ID NO: 93 - protein, 16 aa, Artificial Sequence
  CDR2 Heavy Chain MSM R707WMF mutant
VSAITPRGGYTYYADS

SEQ ID NO: 94 - protein, 18 aa, Artificial Sequence
  CDR3 Heavy Chain MSM R707WMF mutant
CARGLTWMYTPGFDYWGQ

SEQ ID NO: 95 - protein, 12 aa, Artificial Sequence
  CDR1 Heavy Chain MSM R707SMF mutant
FTFSNYAIHWVR

SEQ ID NO: 96 - protein, 16 aa, Artificial Sequence
  IgG4 CDR2 Heavy Chain MSM R707SMF mutant
VSAITPRGGYTYYADS

SEQ ID NO: 97 - protein, 18 aa, Artificial Sequence
  CDR3 Heavy Chain MSM R707SMF mutant
CARGLTSMYTPGFDYWGQ

SEQ ID NO: 98 - protein, 12 aa, Artificial Sequence
  CDR1 Heavy Chain MSM R707TMF mutant
FTFSNYAIHWVR

SEQ ID NO: 99 - protein, 16 aa, Artificial Sequence
  CDR2 Heavy Chain MSM R707TMF mutant
VSAITPRGGYTYYADS

SEQ ID NO: 100 - protein, 18 aa, Artificial Sequence
  CDR3 Heavy Chain MSM R707TMF mutant
CARGLTTMYTPGFDYWGQ

SEQ ID NO: 101 - protein, 12 aa, Artificial Sequence
  CDR1 Heavy Chain MSM R707NMF mutant
FTFSNYAIHWVR

SEQ ID NO: 102 - protein, 16 aa, Artificial Sequence
  CDR2 Heavy Chain MSM R707NMF mutant
VSAITPRGGYTYYADS

SEQ ID NO: 103 - protein, 18 aa, Artificial Sequence
  CDR3 Heavy Chain MSM R707NMF mutant
CARGLTNMYTPGFDYWGQ

SEQ ID NO: 104 - protein, 12 aa, Artificial Sequence
  CDR1 Heavy Chain MSM R707KMF mutant
FTFSNYAIHWVR

SEQ ID NO: 105 - protein, 16 aa, Artificial Sequence
  CDR2 Heavy Chain MSM R707KMF mutant
VSAITPRGGYTYYADS

SEQ ID NO: 106 - protein, 18 aa, Artificial Sequence
  CDR3 Heavy Chain MSM R707KMF mutant
CARGLTKMYTPGFDYWGQ

SEQ ID NO: 107 - protein, 12 aa, Artificial Sequence
  CDR1 Heavy Chain MSM R707RMF mutant
FTFSNYAIHWVR

SEQ ID NO: 108 - protein, 16 aa, Artificial Sequence
  CDR2 Heavy Chain MSM R707RMF mutant
VSAITPRGGYTYYADS

SEQ ID NO: 109 - protein, 18 aa, Artificial Sequence
  CDR3 Heavy Chain MSM R707RMF mutant
CARGLTRMYTPGFDYWGQ

SEQ ID NO: 110 - protein, 12 aa, Artificial Sequence
  CDR1 Heqavy Chain MSM R707MLF mutant
FTFSNYAIHWVR

SEQ ID NO: 111 - protein, 16 aa, Artificial Sequence
  CDR2 Heavy Chain MSM R707MLF
VSAITPRGGYTYYADS

SEQ ID NO: 112 - protein, 18 aa, Artificial Sequence
  CDR3 Heavy Chain MSM R707MLF
CARGLTMLYTPGFDYWGQ

SEQ ID NO: 113 - protein, 12 aa, Artificial Sequence
  CDR1 Heavy Chain MSM R707MVF
FTFSNYAIHWVR

SEQ ID NO: 114 - protein, 16 aa, Artificial Sequence
  CDR2 Heavy Chain MSM R707MVF mutant
VSAITPRGGYTYYADS

SEQ ID NO: 115 - protein, 18 aa, Artificial Sequence
  CDR3 Heavy Chain MSM R707MVF mutant
CARGLTMVYTPGFDYWGQ

SEQ ID NO: 116 - protein, 12 aa, Artificial Sequence
  CDR1 Heavy Chain MSM R707MAF mutant
FTFSNYAIHWVR

SEQ ID NO: 117 - protein, 16 aa, Artificial Sequence
  CDR2 Heavy Chain MSM R707MAF mutant
VSAITPRGGYTYYADS

SEQ ID NO: 118 - protein, 18 aa, Artificial Sequence
  CDR3 Heavy Chain MSM R707MAF mutant
CARGLTMAYTPGFDYWGQ

SEQ ID NO: 119 - protein, 12 aa, Artificial Sequence
  CDR1 Heavy Chain MSM R707MGF mutant
FTFSNYAIHWVR

SEQ ID NO: 120 - protein, 16 aa, Artificial Sequence
  CDR2 Heavy CHain MSM R707MGF mutant
VSAITPRGGYTYYADS

SEQ ID NO: 121 - protein, 18 aa, Artificial Sequence
  CDR3 Heavy CHain MSM R707MGF mutant
CARGLTMGYTPGFDYWGQ

SEQ ID NO: 122 - protein, 12 aa, Artificial Sequence
  CDR1 Heavy Chain MSM R707MSF mutant
FTFSNYAIHWVR

SEQ ID NO: 123 - protein, 16 aa, Artificial Sequence
  CDR2 Heavy Chain MSM R707MSF mutant
VSAITPRGGYTYYADS

SEQ ID NO: 124 - protein, 18 aa, Artificial Sequence
  CDR3 Heavy Chain MSM R707MSF mutant
CARGLTMSYTPGFDYWGQ

SEQ ID NO: 125 - protein, 12 aa, Artificial Sequence
  CDR1 Heavy Chain MSM R707MTF mutant
FTFSNYAIHWVR

SEQ ID NO: 126 - protein, 16 aa, Artificial Sequence
  Human CDR2 HC AA MSM R707MTF mutant
VSAITPRGGYTYYADS

SEQ ID NO: 127 - protein, 18 aa, Artificial Sequence
  CDR3 Heavy Chain MSM R707MTF mutant
CARGLTMTYTPGFDYWGQ

SEQ ID NO: 128 - protein, 12 aa, Artificial Sequence
  CDR1 Heavy Chain MSM R707MQF mutant
FTFSNYAIHWVR

SEQ ID NO: 129 - protein, 16 aa, Artificial Sequence
  CDR2 Heavy Chain MSM R707MQF mutant
VSAITPRGGYTYYADS

SEQ ID NO: 130 - protein, 18 aa, Artificial Sequence
  CDR3 Heavy Chain MSM R707MQF muitant
CARGLTMQYTPGFDYWGQ

SEQ ID NO: 131 - protein, 12 aa, Artificial Sequence
  CDR1 Heavy Chain MSM R707MPF mutant
FTFSNYAIHWVR

SEQ ID NO: 132 - protein, 16 aa, Artificial Sequence
  CDR2 Heavy Chain MSM R707MPF mutant
VSAITPRGGYTYYADS

SEQ ID NO: 133 - protein, 18 aa, Artificial Sequence
  CDR3 Heavy Chain MSM R707MPF mutant
CARGLTMPYTPGFDYWGQ

SEQ ID NO: 134 - protein, 12 aa, Artificial Sequence
  CDR1 Heavy Chain MSM R707MRF mutant
FTFSNYAIHWVR

SEQ ID NO: 135 - protein, 16 aa, Artificial Sequence
  CDR2 Heavy Chain MSM R707MRF mutant
VSAITPRGGYTYYADS

SEQ ID NO: 136 - protein, 18 aa, Artificial Sequence
  CDR3 Heavy Chain MSM R707MRF mutant
CARGLTMRYTPGFDYWGQ

SEQ ID NO: 137 - protein, 12 aa, Artificial Sequence
  CDR1 Heavy Chain MSM R707B mutant
FTFSNYAIHWVR

SEQ ID NO: 138 - protein, 16 aa, Artificial Sequence
  CDR2 Heavy Chain MSM R707B mutant
VSAITPRGGYTYYADS

SEQ ID NO: 139 - protein, 18 aa, Artificial Sequence
  CDR3 Heavy Chain MSM R707B mutant
CARGLTYSYTPGFDYWGQ

SEQ ID NO: 140 - protein, 12 aa, Artificial Sequence
  CDR2 Heavy Chain MSM R707BI mutant
FTFSNYAIHWVR

SEQ ID NO: 141 - protein, 16 aa, Artificial Sequence
  CDR2 Heavy Chain MSM R707BI mutant
VSAITPRGGYTYYADS

SEQ ID NO: 142 - protein, 18 aa, Homo sapiens
  CDR3 Heavy Chain MSM R707BI mutant
CARGLTISYTPGFDYWGQ

SEQ ID NO: 143 - protein, 12 aa, Artificial Sequence
  CDR1 Heavy Chain MSM R707BL mutant
FTFSNYAIHWVR

SEQ ID NO: 144 - protein, 16 aa, Artificial Sequence
  CDR2 Heavy Chain MSM R707BL mutant
VSAITPRGGYTYYADS

SEQ ID NO: 145 - protein, 18 aa, Artificial Sequence
  CDR3 Heavy Chain MSM R707BL mutant
CARGLTLSYTPGFDYWGQ

SEQ ID NO: 146 - protein, 12 aa, Artificial Sequence
  CDR1 Heavy Chain MSM R707BR mutant
FTFSNYAIHWVR

SEQ ID NO: 147 - protein, 16 aa, Artificial Sequence
  CDR2 Heavy Chain MSM R707BR mutant
VSAITPRGGYTYYADS

SEQ ID NO: 148 - protein, 18 aa, Artificial Sequence
  CDR3 Heavy Chain MSM R707BR mutant
CARGLTRSYTPGFDYWGQ

SEQ ID NO: 149 - protein, 12 aa, Homo sapiens
  CDR1 Light Chain VK3 A27 germline
RASQSVSSSYLA

SEQ ID NO: 150 - protein, 7 aa, Homo sapiens
  CDR2 Light Chain VK3 A27 germline
GASSRAT

SEQ ID NO: 151 - protein, 13 aa, Homo sapiens
  CDR3 Light Chain VK3 A27 germline
CQQSYSSPITFGQ

SEQ ID NO: 152 - protein, 12 aa, Artificial Sequence
  CDR1 Light Chain MSM R707 germline
RASQSVSSSYLA

SEQ ID NO: 153 - protein, 7 aa, Artificial Sequence
  CDR2 Light Chain MSM R707 germline
GASSRAT

SEQ ID NO: 154 - protein, 13 aa, Artificial Sequence
  CDR3 Light Chain MSM R707 germline
CQQSYSSPITFGQ

SEQ ID NO: 155 - protein, 378 aa, Homo sapiens; identical to SEQ ID NO: 73
  Chemokine Receptor 7
  Chemokine Receptor CCR7

SEQ ID NO: 156 - protein, 381 aa, Macaca fascicularis
  Chemokine Receptor CCR7
CYNMDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSGLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 157 - protein, 378 aa, Callithrix jacchus
  Chemokine Receptor CCR7
MDLGKPMKRVLVVALLVIFQVCMCQDEVTDDYIGDNTTVDYTLFETLCSKRDVRNFKAWFLPVMYSFICFVGLLGNGLVVLTYIYFKRLKTMTDTYLLNLAVADILFLLTLPFWAYSAAKSWAFGVHLCKLIFGIYKMSFFSGMLLLLCISIDRYVAIVQAVSAHRHRARVLLISKLSCVGIWVLATVLSIPEVLYSGLQKSSSEQAMRCSLITEHVEAFITIQVAQMVIGFLVPLLAMSFCYLVIIRTLLQARNFERNKAIKVIIAVVVVFIIFQLPYNGVVLAQTVANFNITSSSCELSKQLNIAYDITYSLACVRCCVNPFLYAFIGVKFRNDLFKLFKDLGCLSQERLRQWSSCRHARRSSMSVEAETTTTFSP

SEQ ID NO: 158 - protein, 378 aa, Mus musculus
  Chemokine Receptor 7
  Chemokine Receptor CCR7
MDPGKPRKNVLVVALLVIFQVCFCQDEVTDDYIGENTTVDYTLYESVCFKKDVRNFKAWFLPLMYSVICFVGLLGNGLVILTYIYFKRLKTMTDTYLLNLAVADILFLLILPFWAYSEAKSWIFGVYLCKGIFGIYKLSFFSGMLLLLCISIDRYVAIVQAVSAHRHRARVLLISKLSCVGIWMLALFLSIPELLYSGLQKNSGEDTLRCSLVSAQVEALITIQVAQMVFGFLVPMLAMSFCYLIIIRTLLQARNFERNKAIKVIIAVVVVFIVFQLPYNGVVLAQTVANFNITNSSCETSKQLNIAYDVTYSLASVRCCVNPFLYAFIGVKFRSDLFKLFKDLGCLSQERLRHWSSCRHVRNASVSMEAETTTTFSP

SEQ ID NO: 159 - protein, 98 aa, Homo sapiens
  Chemokine CCL19
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 160 - protein, 98 aa, Macaca fascicularis
  Chemokine CCL19
MALLLALSLLVLWTSPAPTLSGTNDAEDCCLSVTQKPIPGYIVRNFRYLLIKDGCRVPAVVFTTLRGRQLCAPPDQSWVERIIQRLQRTSTKMKRRSS

SEQ ID NO: 161 - protein, 98 aa, Macaca mulatta; identical to SEQ ID NO: 160
  Chemokine CCL19

SEQ ID NO: 162 - protein, 98 aa, Callithrix jacchus
  Chemokine CCL19
MALLLALSLLVLWTSPAPTLSGTNDAEDCCLSVTQNPIPGYIIRNFRYLLIKDGCRVPAVVFTTVRGRQLCAPLDQPWVERIIRRLQRTSAKMKRRSS

SEQ ID NO: 163 - protein, 107 aa, Mus musculus
  Chemokine CCL19
MAPRVTPLLAFSLLVLWTFPAPTLGGANDAEDCCLSVTQRPIPGNIVKAFRYLLNEDGCRVPAVVFTTLRGYQLCAPPDQPWVDRIIRRLKKSSAKNKGNSTRRSPV

SEQ ID NO: 164 - protein, 134 aa, Homo sapiens
  Chemokine CCL21
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 165 - protein, 131 aa, Macaca fascicularis
  Chemokine CCL21
MAQSLALSLLILVLAFGIPGTQGSDGGAQDCCLKYSQRKIPAKVVRSYRKQEPSLGCSIPAILFLPRKRSQAELCADPKELWVQQLMQHLDKTPTPRKPVQGCRKDRGVPKNGKKGKGCKRTEQSQTPKGP

SEQ ID NO: 166 - protein, 131 aa, Macaca mulatta; identical to SEQ ID NO: 165
  Chemokine Receptor CCL21

SEQ ID NO: 167 - protein, 134 aa, Callithrix jacchus
  Chemokine CCL21
MAQSLALSLLILVLAFGIPRTQGSDGGAQDCCLRYSLRKIPAKVVRGYRKQEPSLGCSIPAILFLPRKRSQPELCADPKEPWVQQLMQHLDKTPAPRKPPQNCRKDRGAPKTGKKGKGSKGCKRTEQSQTPKGP

SEQ ID NO: 168 - protein, 133 aa, Mus musculus
  Chemokine CCL21
MAQMMTLSLLSLDLALCIPWTQGSDGGGQDCCLKYSQKKIPYSIVRGYRKQEPSLGCPIPAILFLPRKHSKPELCANPEEGWVQNLMRRLDQPPAPGKQSPGCRKNRGTSKSGKKGKGSKGCKRTEQTQPSRG